(19) United States
(12) Patent Application Publication
(10) Pub. No.: US 2007/0099828 A1
(43) Pub. Date: May 3, 2007

(54) BIOMARKERS FOR THE EFFICACY OF CALCITONIN AND PARATHYROID HORMONE TREATMENT

(76) Inventor: Maria Bobadilla, HESINGUE (FR)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/580,779

(22) PCT Filed: Nov. 24, 2004
(86) PCT No.: PCT/EP04/13347

§ 371 Date: May 25, 2006

Related U.S. Application Data
(60) Provisional application No. 60/525,025, filed on Nov. 25, 2003.

(51) Int. Cl. A61K 38/23

(52) U.S. Cl. 514/12

(57) ABSTRACT

A mufti-organ gene profiling analysis of the results of an administration to a subject of salmon calcitonin or a parathyroid hormone analogue provides biomarkers of calcitonin treatment efficacy and parathyroid hormone or parathyroid hormone analogue treatment efficacy. Among the biomarkers are the expression profiles of the genes for Y-box binding protein, BMPs, FGFs, IGFs, VEGF, &x3B1;-2-HS glycoprotein (AHSG), OSF, nuclear receptors (steroid/thyroid family) and others. The results obtained support the anabolic effect of salmon calcitonin on bone metabolism.

### FIELD OF THE INVENTION

This invention relates generally to the analytical testing of tissue samples in vitro, and more particularly to aspects of gene expression profiling concerning calcium regulation.

### BACKGROUND OF THE INVENTION

Calcium is essential for many cellular processes in the body and especially important for bone metabolism. The level of calcium in the body is carefully maintained by an endocrine control system. Two of the hormones in this endocrine control system are calcitonin and parathyroid hormone.

Calcitonins, which are polypeptide hormones of about 32 amino acids, are endogenous regulator of calcium homeostasis and can be used as anti resorptive agents for the treatment of hypocalcaemia-associated disorders. Calcitonin is produced in the parafollicular cells (C cells) of the thyroid gland. Various calcitonins, including e.g. salmon and eel calcitonin, are commercially available and are commonly employed in the treatment of e.g. Paget's disease of bone, malignant hypocalcaemia and post-menopausal osteoporosis. Pondel M, Intl. J. Exp. Pathol. 81(6): 405-22 (2000). A version of calcitonin (Miacalcin®) is available as a nasal spray.

Parathyroid hormone (PTH) is a polypeptide of 84 amino acids. Parathyroid hormone regulates bone remodelling and Ca²⁺ homeostasis. Parathyroid hormone is also a known paracrine activator of osteoclast differentiation and activity. PTS893 [SDZ PTS 893; Leu8, Asp10, Lys11, Ala16, Gln18, Thr33, Ala34 human PTH 1-34 [hPTH(1-34)]]is a 34 amino acid parathyroid analogue that enhances bone mass and biomechanical properties. Kneissel M et al., Bone 28: 237-50 (March 2001); Stewart A F et al., J. Bone. Miner. Res. 15(8): 1517-25 (August 2000); Thomsen J S et al., Bone 25(5):561-9 (November 1999).

Calcitonin and parathyroid hormone are known to interact in a complex and interdependent manner, but the understanding of how calcitonin and parathyroid hormone interact has been incomplete. Calcitonin inhibitory effects on osteoclast resorptive activity, and renal tubular calcium resorption have been well documented. However, potential calcitonin effects on osteoblasts and interactions with any other skeletal-metabolism-related factors have remained controversial.

Multi-organ gene profiling analysis would provide a better picture of the changes induced by a compound on the whole organism and also give a new perspective to the understanding of the pharmacology of hormones. Genomics technologies are a source of the new hypothesis-generating capabilities that are now empowering biomedical researchers. In the context of drug development, they provide with a new perspective to the understanding of the pharmacology of drugs. Accordingly, there is a need in the art for an organism-wide understanding of the activity of calcitonin and parathyroid hormone.

### SUMMARY OF THE INVENTION

The invention provides a response to the need in the art. Multi-organ gene profiling analysis provides with a complete picture of the changes induced by a compound on the whole organism, and gives a new perspective to the understanding of the pharmacology of drugs. In one aspect, the invention provides the first description of the molecular mechanisms of action of hormonal-mediated bone remodelling by salmon calcitonin by gene profiling analysis. The known mechanisms of action of calcitonin as anti-resorptive agent could be reconstructed at the molecular level. Effects on effectors and pathways linked to bone remodelling activities—BMPs, IGFs, extracellular matrix components and VEGF—were also observed. These results support the role of calcitonin as an anabolic agent. In another aspect, the invention provides the first reconstruction of the molecular mechanisms of action of a pharmacological agent on one of its target tissues in an intact primate animal model, by evaluating the gene expression changes induced by salmon calcitonin or the parathyroid hormone analogue PTS893 on bone in cynomolgus monkeys, to elucidate the molecular mechanisms of action mediating their effects. Gene profiling analysis allowed the reconstruction of the pathways involved in calcitonin signal transduction, triggered by protein-G-linked-receptor stimulation and their influence on cell cycle, as indicated by the changes observed in yclins. In vivo gene-profiling expression studies allow the identification of the molecular mechanisms underlying a pharmacological effect.

In one embodiment, the invention provides for the use of calcitonin in the manufacture of a medicament for the treatment of a condition for which treatment with an anabolic agent is indicated. In one embodiment, the condition is atherosclerosis.

The invention also provides for the use of calcitonin in the manufacture of a medicament for the treatment of disorders of calcium metabolism in a selected patient population, where the patient population is selected on the basis of the gene expression profile indicative of calcitonin efficacy by the patient to whom calcitonin is administered. In one embodiment, the calcitonin is salmon calcitonin. The invention fuirther provides for the use of a parathyroid hormone or parathyroid hormone analogue in the manufacture of a medicament for the treatment of disorders of calcium metabolism in a selected patient population, where the patient population is selected on the basis of the gene expression profile indicative of parathyroid hormone or parathyroid hormone analogue efficacy by the patient to whom parathyroid hormone or parathyroid hormone analogue is administered. In one embodiment, the hormone analogue is PTS893. In one embodiment, the medicament is administered in a therapeutic dose prior to determining the gene expression profile by the patient. In another embodiment, the medicament is administered in a sub-therapeutic dose prior to determining the gene expression profile by the patient.

The invention also provides a method for treating a condition in a subject, wherein the condition is one for which administration of a calcitonin, parathyroid hormone, a parathyroid hormone analogue or a combination thereof is indicated. The method involves, first administering a compound of interest to the subject (e.g., a primate subject) and then obtaining the gene expression profile of the subject following administration of the compound. The gene expression profile of the subject is compared to a biomarker gene expression profile. The biomarker gene expression profile is indicative of efficacy of treatment by a calcitonin, parathyroid hormone, a parathyroid hormone analogue or a combination thereof In one embodiment, the biomarker gene expression profile is the baseline gene expression profile of the subject before administration of the compound. In another embodiment, the biomarker gene expression profile is the gene expression profile or average of gene expression profiles of a vertebrate to whom calcitonin (e.g., salmon calcitonin) or parathyroid hormone or a parathyroid hormone analogue (e.g., PTS893) has been administered. A similarity in the gene expression profile of the subject to whom the compound was administered to the biomarker gene expression profile is indicative of efficacy of treatment with the compound.

Accordingly, the invention provides biomarkers for the efficacy of treatment of a condition for which calcitonin, parathyroid hormone or a combination thereof is indicated. Among the biomarkers are the expression profiles of the genes for Y-box binding protein, bone morphogenetic proteins (BMPs), fibroblast growth factors (FGFs), insulin-like growth factors (IGFs), vascular endothelial growth factor (VEGF), α-2-HS glycoprotein (AHSG), osteoclast stimulating factor (OSF), nuclear receptors (steroid/thyroid family) and others.

The invention provides methods for determining a subject for inclusion in a clinical trial, based upon an analysis of biomarkers expressed in the subject to be treated. The compound to be tested is administered to the subject. In one embodiment, the compound to be tested is administered in a sub-therapeutic dose. Then, the gene expression profile of the subject following administration of the compound is obtained. The subject may be included in the clinical trial when the gene expression profile of the subject to whom the compound was administered is similar to a biomarker gene expression profile indicative of efficacy of treatment by a calcitonin, parathyroid hormone, a parathyroid hormone analogue or a combination thereof. The subject may be excluded from the clinical trial when the gene expression profile of the subject is dissimilar to the biomarker gene expression profile indicative of efficacy of treatment. Such similarities or dissimilarities are observable to those of skill in the art.

The invention also provides clinical assays, kits and reagents for determining treatment efficacy of a condition for which administration of a calcitonin, parathyroid hormone or a parathyroid hormone analogue is indicated. In one embodiment, the kits contain reagents for determining the gene expression of biomarker genes, by hybridization. In another embodiment, the kits contain reagents for determining the gene expression of biomarker genes, by the polymerase chain reaction.

### DESCRIPTION OF THE PREFERRED EMBODIMENTS

This invention is based upon an understanding of the effects of administration to a subject of calcitonin (e.g., salmon calcitonin; SEQ ID NO:1) or parathyroid hormone (SEQ ID NO:2) or an analogue thereof (e.g., PTS893; SEQ ID NO:3). A multi-organ gene profiling analysis of the results of an administration to a subject of salmon calcitonin or a parathyroid hormone analogue provides biomarkers of calcitonin treatment efficacy and parathyroid hormone or parathyroid hormone analogue treatment efficacy. As used herein, a subject is a vertebrate. In one embodiment, the vertebrate is a mammal. In a more particular embodiment, the subject is a primate, e.g., a cynomolgus money or a human.

The analysis provided here globally describes the molecular mechanisms of action of salmon calcitonin and the PTS893 in changing the ribonucleic acid (RNA) content in different organs by multi-organ gene profiling analysis in primates. The RNA content of the cell, the “transcriptome” is a reflection of the cell functions and status. Inside an individual cell or an organ, the expressions of the different elements of a transcriptome are not independent. The change in expression level can trigger a series of events that will lead finally to another modification of the transcriptome. These interdependent events are described in terms of pathways. Because the changes in the different functions inside a cell are tightly interconnected, the changes in different organs inside the organism are linked. Applying gene profiling to different organs submitted to the same treatment gives an improved overview of the effects and the modifications of the physiological status. As shown herein, this is particularly the situation when multi-organ profiling analysis of pleiotropic compounds, such as calcitonin, is to be performed. Indeed, the global signature described for calcitonin is reflected not only in the main target organ (i.e., bone) but also in the other organs analyzed herein.

In this multi-organ gene profiling analysis, the known mechanisms of action of calcitonin as an anti-resorptive agent and the parathyroid hormone PTS893 as a paracrine activator of osteoclast differentiation and activity could be reconstructed at the molecular level. The calcitonin inhibitory effect on osteoclasts could be reconstructed, with changes affecting, among others, genes for PU.1 (SPI1; SpiB; SEQ ID NO:4), colony stimulating factor (CSF-1 (SEQ ID NO:6); differentiation and survival) carbonic anhydrase (SEQ ID NO:8), H⁺-ATPases, cathepsin K (resorptive activity) tubulins, PAK4 (motility). Effects on effectors and pathways linked to bone remodelling activities (bone morphogenetic proteins (BMPs), fibroblast growth factors (FGFs), insulin-like growth factors (IGFs), extracellular matrix components, steroid hormones, vascular endothelial growth factor (VEGF) and α-2-HS glycoprotein (AHSG)) were also observed, shared in many cases by both salmon calcitonin and PTS893. Interestingly, salmon calcitonin also regulates the expression of the gene codifying for osteoclast stimulating factor (OSF), and cystatin. Also interestingly, PTS893 also regulates the genes implicated in osteoclast differentiation and survival (SPI1, CSF-1, monocyte to macrophage differentiation-associated protein (1)). PTS893 also produced a strong up-regulation on nuclear receptors (steroid/thyroid family). Accordingly, these results support the role of calcitonin as anabolic agent.

Calcitonin is presently used in the treatment of systemic skeletal diseases characterized by high bone mass which are a consequence of imbalance between bone formation (anabolic) and resorption of bone, with the former predominating. Calcitonin promotes the synthesis of bone morphogenetic protein-2 (BMP-2), which is known to be a potent anabolic agent. The evidence is strong that when calcitonin gets to bone cells, they can have an anabolic effect by increasing production of BMP-2. Thus calcitonin can be used in a method of treating an individual to adjust a subject's bone minneral density.

This the first approach to characterise in an in vivo model the effects of calcitonin on bone metabolism by gene expression profiling. The calcitonin inhibitory effect on osteoclasts could be reconstructed, with changes affecting genes as carbonic anhydrase, H⁺-AThases and cathepsin K. Salmon calcitonin also seemed to regulate the expression of the gene codifying for cystatin, being this effect described here for the first time. Salmon calcitonin has also modulating effects on genes affecting the direct, autocrine, paracrine and endocrine regulation of the mesenchymal cell functions such as pleiothropin, periostin, fibroblast growth factor, transforming growth factor betas (TGF-betas), insulin-like growth factors/binding proteins (IGFs/IGFBPs), bone morphogenetic proteins (BMPs), Vascular Endothelial Growth Factor (VEGF), Tumour Necrosis Factor (TNF), neurochondrin, follistatin-like 3, or parathyroid hormone receptor. It also regulates the synthesis and degradation of extracellular matrix components (collagens, osteopontin, osteocalcin, dermatopontin, chondroadherin, glypican or syndecan) and enzymes. Salmon calcitonin also influenced some aspects of bone mineralization, since changes in dentin were observed.

As provided herein, calcitonin can also be used as an anabolic agent in the treatment of other conditions where anabolism or tissue growth is therapeutically desirable. Such a condition is atherosclerosis, an atheromatous disease in which the atheromatous plaque is complicated by fibrosis and calcification.

Moreover, the invention provides biomarkers of the efficacy of calcitonin or parathyroid hormone treatment. As used herein, a gene expression profile is diagnostic for determining the efficacy of treatment when the increased or decreased gene expression is an increase or decrease (e.g., at least a 1.5-fold difference) over the baseline gene expression following administration of the compound (i.e., the biomarker gene expression profile is the baseline gene expression profile of the subject before administration of the compound). Alternatively or in addition, the gene expression profile is diagnostic for determining the efficacy of treatment as compared with treatment of calcitonin (e.g., satmon calcitonin) or parathyroid hormone or parathyroid hormone analogues (e.g., PTS893) when the gene expression profile of the treated subject is comparable to a standard biomarker gene expression profile. In one embodiment, the standard biomarker gene expression profile is the gene expression profile or average of gene expression profiles of a vertebrate to whom a calcitonin, parathyroid hormone, a parathyroid hormone analogue of a combination thereof has been administered, this profile or profile being the standard to which the results from the subject following administration is compared. Such an approach, which contains aspects of therapeutics and diagnostics, is termed “theranostic” by many of those of skill in the art.

In one embodiment, the subject is a vertebrate. In a particular embodiment, the vertebrate is a mammal. In a more particular embodiment, the mammal is a primate, such as a cynomolgus monkey or a human. As used herein, the administration of an agent or drug to a subject or patient includes self-administration and the administration by another.

As used herein, a gene expression profile is diagnostic of the efficacy of calcitonin or parathyroid hormone treatment when the increased or decreased gene expression is an increase or decrease (e.g., at least a 1.5-fold difference) over the baseline gene expression following administration of a calcitonin or of parathyroid hormone or an analogue. As used herein, a gene expression pattern is “higher than normal” when the gene expression (e.g., in a sample from a treated subject) shows a 1.5-fold difference (i.e., higher) in the level of expression compared to the baseline samples. A gene expression pattern is “lower than normal” when the gene expression (e.g., in a sample from a treated subject) shows a 1.5-fold difference (i.e., lower) in the level of expression compared to the baseline samples.

Techniques for the detection of gene expression of the genes described by this invention include, but are not limited to northern blots, RT-PCT, real time PCR, primer extension, RNase protection, RNA expression profiling and related techniques. Techniques for the detection of gene expression by detection of the protein products encoded by the genes described by this invention include, but are not limited to, antibodies recognizing the protein products, western blots, immunofluorescence, immunoprecipitation, ELISAs and related techniques. These techniques are well known to those of skill in the art. Sambrook J et al., Molecular Cloning: A Laboratory Manual, Third Edition (Cold Spring Harbor Press, Cold Spring Harbor, 2000). In one embodiment, the technique for detecting gene expression includes the use of a gene chip. The construction and use of gene chips are well known in the art. See, U.S. Pat Nos. 5,202,231; 5,445,934; 5,525,464; 5,695,940; 5,744,305; 5,795,716 and 5,800,992. See also, Johnston, M. Curr Biol 8:R171-174 (1998); Iyer VR et al., Science 283:83-87 (1999) and Elias P, “New human genome ‘chip’ is a revolution in the offing” Los Anigeles Daily News (Oct. 3, 2003).

The gene expression profile may include one or more genes selected from the group of acid phosphatase 1 isoform a; activin A receptor type II like 1; activin A type IIB receptor precursor; activin beta C chain; alpha 2 HS glycoprotein; amelogenin; annexin V; arylsulfatase E precursor; ATPase H(+) vacuolar; ATPase H(+) vacuolar subunit; AITase, H+ transport, lysosomal; ATPase, H+ transporting, lysosomal; ATPase, H+ transporting, lysosomal; biglycan; bone morphogenetic protein 1; bone morphogenetic protein 10; bone morphogenetic protein 2A; bone morphogenetic protein 5; bone morphogenetic protein 6 precursor; calcium binding protein 1 (calbrain); calcium/caimodulin dependent protein kinase (CaM Idnase) II gamma; calreticulin; cAMP responsive element modulator (CREM); carbonic anhydrase I; carbonic anhydrase II; cartilage oligomeric matrix protein precursor; cathepsin K; cathepsin W; CDC like kinase 1; CDC like kinase 2 isoform hclk2/139; chondroitin sulphate proteoglycan 2 (versican); chondroitin sulphate proteoglycan 3 (neurocan); chorionic somatomammotropin hormone 1; chymotrypsin C (caldecrin); collagen type 1 and PDGFB fusion transcript; collagen type II alpha 1; collagen type III alpha 1; collagen type IV alpha 2; collagen type IX alphal; collagen type VI alpha 1; collagen type VI alpha 2 (AA 570 998); collagen type XI alpha 1; collagen type XI alpha2; collagen type XI alpha2; collagen, type I, alpha 2; collagen, type IV, alpha 1; collagen, type IX, alpha 2; collagen, type V, alpha 2; collagen, type VI, alpha 1; collagen, type VI, alpha 1 precursor; collagen, type XVI, alpha 1; collagen, type XVI alpha 1; collagenase 3 (matrix metalloproteinase 13); connective tissue growth factor; cyclin A2; cyclin B1; cyclin D2; cyclin E2; cyclin dependent kinase 5; cyclin dependent kinase 5, regulatory subunit 1 (p35); cyclin dependent linase 6; cyclin dependent kinase inhibitor 1A (p21, Cip1); cystatin B (stefin B); cytokine inducible kinase; death associated protein kinase 1; death associated protein kinase 3; dentin matrix acidic phosphoprotein 1 (DMP); dual specificity phosphatase 9; dystrophia myotonica protein kinase; ectonucleotide pyrophosphatase/phosphodiesterase 1; ectonucleotide pyrophosphatase/phosphodiesterase 1; endothelial differentiation, G protein coupled receptor 6 precursor; oestrogen receptor; oestrogen receptor; oestrogen receptor related protein; oestrogen responsive B box protein (EBBP); fibroblast activation protein; fibroblast growth factor 1 (acidic); fibroblast growth factor 18; fibroblast growth factor 4; fibroblast growth factor receptor; follistatin like 1; follistatin like 1; glutamate receptor, metabotropic 1; GPI1 N acetylglucosaminyl transferase component Gpi1; granulocyte macrophage colony stimulating factor (CSF1); growth arrest and DNA damage inducible, alpha; growth factor receptor bound protein 10; heparan sulphate proteoglycan 2 (perlecan); inositol 1,4,5 triphosphate receptor, type 1; inositol 1,4,5 triphosphate receptor, type 1; inositol 1,4,5 triphosphate receptor, type 2; inositol 1,4,5 trisphosphate 3 kinase isoenzyme; inositol polyphosphate 4 phosphatase type I beta; inositol polyphosphate 5 phosphatase; inositol(myo) 1(or 4) monophosphatase 1; inositol(myo) 1(or 4) monophosphatase 2; insulin like growth factor (IGF II); insulin like growth factor 2 (somatomedin A); insulin like growth factor binding protein; insulin like growth factor binding protein 2; insulin like growth factor binding protein 3; insulin like growth factor binding protein 5; insulin like growth factor binding protein 2; insulin like growth factor II precursor; insulin like growth factor II precursor; integrin alpha 10 subunit; interleukin 1 receptor associated kinase; Janus kinase 3; LIM protein (similar to rat protein kinase C binding enigma); lysyl oxidase like protein; MAD, mothers against decapentaplegic homolog 3; MAGUKs (membrane associated guanylate kinase homologues; MAP kinase kinase kinase (MTK1); MAPK13: mitogen activated protein kinase 13; MAPK8IP1: mitogen activated protein kinase 8 interacting protein 1; MEK kinase; metalloproteinase; mitogen activated protein kinase 1; mitogen activated protein kinase 8; mitogen activated protein kinase kinase 1; mitogen activated protein kinase kinase kinase kinase 4; mitogen activated protein kinase activated protein kinase 2; mitogen activated protein kinase activated protein kinase 3; MMD: monocyte to macrophage differentiation associated; neurochondrin; nuclear factor of activated T cells, cytoplasmic, calcineurin dependent 1; OS 4 protein (OS 4); OSF 2os osteoblast specific factor 2 (periostin); osteoclast stimulating factor (OSF); PAK4; PDGF associated protein; phosphatidylinositol 4 kinase, catalytic, beta polypeptide; phosphatidylinositol glycan, class L; phosphatidylinositol polyphosphate 5 phosphatase, isoform b; phosphatidylinositol 4 phosphate 5 kinase isoform C (1); phosphatidylinositol 4 phosphate 5 kinase, type I, beta; phosphatidylinositol 4 phosphate 5 kinase, type II, beta; phosphatidylinositol glycan class C (PIG C); phosphodiesterase 4A, cAMP specific; phosphodiesterase 4D, cAMP specific (dunce (Drosophila) homolog phosphodiesterase E3); phosphodiesterase B, calmodulin dependent; phosphoinositide 3 kinase; phosphoinositide 3 kinase, catalytic, gamma polypeptide; phosphoinositide 3 kinase, class 3; phospholipase C b3; phospholipase C, beta 4; phospholipase D; phosphotidylinositol transfer protein; PKD2 Protein kinase D2; preprocollagen type I alpha 2; preprocollagen type I alphal; procollagen alpha 1 type II; pro collagen lysine 5 dioxygenase; procollagen proline, 2 oxoglutarate 4 dioxygenase (proline 4 hydroxylase), alpha polypeptide I; progestagen associated endometrial protein (placental protein 14, pregnancy associated endometrial alpha 2 globulin, alpha uterine protein); prolidase (imidodipeptidase) PEPD; proliferating cell nuclear antigen; prolyl 4 hydroxylase beta; protease, serine, 11 I(GF binding); proteasome (prosome, macropain) subunit, beta type, 10; protein inhibitor of activated STAT X; protein kinase 1 PCTAIRE; protein kinase C substrate 80K H; protein kinase C, alpha; protein kinase, cAMP dependent, catalytic, gamma; protein kinase, cAMP dependent, regulatory, type I, beta; protein kinase, cAMP dependent, regulatory, type II, alpha; purinergic receptor P2Y, G protein coupled, 11; RAC2 Ras related C3 botulinum toxin substrate 2 (rho family, small GTP binding protein Rac2); receptor tyrosine kinase DDR; retinoid X receptor gamma; ribosomal protein S6 kinase; ribosomal protein S6 kinase, 90 kD, polypeptide 3; SCAMP1: secretory carrier membrane protein 1 (vesicular transport); secreted phosphoprotein 1 (osteopontin, bone sialoprotein I, early T lymphocyte activation 1); serine (or cysteine) proteinase inhibitor, clade H (heat shock protein 47), member 2; serine/threonine kinase 38; serine/threonine protein kinase; SF 1; Steroidogenic factor 1; signal transducer and activator of transcription 1; signal transducer and activator of transcription 2, 113 kD; signal transducer and activator of transcription 5A; signal transducer and activator of transcription 5A; signal transducer and activator of transcription 6 (STAT6); Smad 3; Smad anchor for receptor activation, isoform 1; Smad5; SMAD6 (inhibits BMP/Smad1 (MADH1); SNF 1 related kinase; SpiB transcription factor (SPI1/PU.1 related); Stat5b (stat5b); Ste20 related serine/threonine kinase; TEIG; TGFB inducible early growth response; TGFB inducible early growth response; TIEG; TGFB1 induced anti apoptotic factor 1; TGF beta induced apoptosis protein 12; TGF beta precursor; TGF beta superfamily protein; Tob; tousled like kinase 1; transforming growth factor, beta receptor III (betaglycan, 300 kD); transforming growth factor beta 3 (TGF beta 3); TRIO: triple functional domain (PTPRF interacting); tubulin alpha 1; tubulin alpha 3; tubulin alpha isotype H2 alpha; tubulin beta 2; tubulin beta 3; tubulin beta 4; tubulin beta, cofactor D; type VI collagen alpha 2 chain precursor; ubiquitin carrier protein E2 C; vascular endothelial growth factor; vascular endothelial growth factor; vascular endothelial growth factor B; and Y box binding protein 1.

As used herein, the administration of an agent or drug to a subject or patient includes self-administration and the administration by another.

Calcitonin. The term “calcitonin” includes not only the naturally occurring calcitonins, but also their pharmaceutically active derivatives and analogues, e.g. in which one or more of the peptide residues present in the naturally occurring product is replaced, or in which the N- or C-terminal is modified. Preferred calcitonins for use in accordance with the invention are salmon, human and porcine calcitonins and Elcatonin. All of these compounds are commercially available and have been extensively described, together with their pharmaceutical properties, in the literature. See, U.S. Pat. Nos. 5,733,569 and 5,759,565, the contents of which are incorporated by reference.

The amount of calcitonin to be administered in accordance with the method of the invention and hence the amount of active ingredient in the composition of the invention depends on the particular calcitonin chosen, the condition to be treated, the desired frequency of administration and the effect desired.

The bioavailability for calcitonins, in particular sahnon calcitonin, as determined in terms of blood plasma concentration following nasal administration is high, generally of the order of ca. 50% of levels achieved on intra-muscular injection. Accordingly administration in accordance with the invention will appropriately be effected so as to give a dosage rate of the order of two times or more, e.g. from about two to four times the dosage rate required for treatment via intra-parietal, e.g. intra-muscular, administration. Information regarding the administration of Miacalcin® (calcitonin-salmon) nasal spray is available in the Miacalcin® Prescribing Iniformation (Novartis, November 2002).

For intra-muscular injection, individual dosages of ca. 50 to 100 MRC units are applied at a rate of from ca. one time daily to ca. three times weekly. For nasal administration in accordance with the present invention, treatment will therefore suitably comprise administration of dosages of from about 50 to about 400 MRC units, more preferably from about 100 to about 200 MRC units at a frequency of from about one time daily to about three times weekly. Conveniently dosages as aforesaid will be administered in a single application, i.e. treatment will comprise administration of single nasal dosages comprising about 50 to about 400 MRC units, preferably about 100 to about 200 MRC units, calcitonin. Alternatively such dosages may be split over a series of e.g. two to four applications taken at intervals during the day, the dosage at each application then comprising about 10 to about 200 MRC units, preferably about 25 to about 100 MRC units.

The total composition quantity administered at each nasal application suitably comprises from about 0.05 to 0.15 ml, typically about 0.1 ml, e.g. 0.09 ml. Compositions for use accordingly suitably comprise from about 150 to about 8,000, preferably from about 500 to about 4,000, more preferably from about 500 to about 2,500, and most preferably from about 1,000 to about 2,000 MRC units calcitonin, e.g. salmon calcitonin, per ml.

The term “calcitonin” also encompasses active peptide analogues and mimetics, such as described for example, in U.S. Pat. Nos. 5,719,122, 5,175,146, and 5,698,6721. See, U.S. Pat. Appln. 2003015815. The “calcitonin superfamily” consists of calcitonin, calcitonin gene-related peptide (CGRP), and amylin. Calcitonin and CGRP derive from the CT/CGRP gene, in humans. Alternative splicing of the primary RNA transcript leads to the translation of CGRP and CT peptides in a tissue-specific manner. CGRP (a 37-amino-acid neuropeptide) and its receptors are widely distributed in the body. Amylin (a 37-amino-acid peptide) is generated from a gene located on chromosome 12 (thought to be an evolutionary duplication of chromosome 11) and shares 46% amino acid sequence homology with CGRP and 20% with human calcitonin. The term “calcitonin gene-related peptide” or “CGRP” includes native CGRP, preferably human CGRP, and its active analogues. CGRP is known to have a variety of roles in bone formation. The term “amylin” includes native amylin, typically from a human source, and its pharmaceutically active analogues. The hormone is known to induce bone-mass formation through a variety of mechanisms. “Calcitonin-like agents” include “calcitonin,” “CGRP,” and “amylin.” See, U.S. Pat. Appln. 003015815.

Paratltyroid hormone. The term “parathyroid hormone” refers to parathyroid hormone, fragments or metabolites thereof and structural analogues thereof which can stimulate bone formation and increase bone mass. Also included are parathyroid hormone related peptides and active fragments and analogues of parathyroid related peptides. See, U.S. Pat. Nos. 4,086,196, 5,001,223, 6,541,450 and 6,649,657 and published PCT patent applications WO 94/01460 and WO 93/06845. Parathyroid hormone functional activity is readily determined by those skilled in the art according to standard assays. A variety of these compounds are described and referenced below, however, other parathyroid hormones will be known to those skilled in the art. Exemplary parathyroid hormones are disclosed in the references cited in U.S. Pat. Nos. 6,541,450 and 6,649,657, the entire contents of which are incorporated by reference. The utility of parathyroid hormones as medical agents in the treatment of conditions which present with low bone mass (e.g., osteoporosis) in mammals is demonstrated by the activity of the parathyroid hormones in conventional assays, including in vivo assays, receptor binding assay, cyclic AMP assays and fracture healing assays.

PTS893 is an analogue of the endogenous parathyroid hormone, in which certain sites of chemical instability are eliminated within N-terminal parathyroid hormone fragments by making appropriate amino acid substitutions at particular residues which results in stable and biologically active human parathyroid hormone fragments. N-terminal fragments of human parathyroid hormones include hPTH(1-34)OH muteins and hPTH(1-38)OH muteins. PTS893 comprises at least the first 27 N-terminal amino acid units of parathyroid hormone. Preferred parathyroid hormone derivatives are those comprising at least one amino acid unit replaced in one or more of the following positions of the parathyroid hormone sequence: 8-11, 13, 16-19, 21, 22, 29 to 34, particularly 8-11, 16-19, 33 and/or 34. These compounds exhibit desirable bone-forming properties both in vivo and in vitro which are equal to or above the level of natural PTH and its N-terminal fragments. See, European patent EP 0 672 057; published PCT patent application WO 94/02510; Kneissel M et al., Bone 28: 237-50 (March 2001); Stewart A F et al., J Bone Miner Res 15(8): 1517-25 (August 2000); Thomsen J S et al., Bone 25(5):561-9 November 1999).

Kits. The kits of the invention may contain a written product on or in the kit container. The written product describes how to use the reagents contained in the kit, e.g., to determine whether a patient is responding effectively or can respond effectively to a compound for use in treating a condition for which calcitonin, parathyroid hormone, a parathyroid hormone analogue or a combination thereof is indicated. In several embodiments, the use of the reagents can be according to the methods of the invention. In one embodiment, the reagent is a gene chip for determining the gene expression of relevant genes.

The following EXAMPLE is presented in order to more fully illustrate the preferred embodiments of the invention. This EXAMPLE should in no way be construed as limiting the scope of the invention, as defined by the appended claims.

### EXAMPLE

### Salmon Calcitonin and PTS893, Pharmacogenomics Exploratory Study in Monkeys; Microarray Gene Expression Analysis

Introduction and summary. The purpose of this EXAMPLE was to evaluate the gene expression changes in cynomolgus monkeys following a two-week subcutaneous treatment with salmon calcitonin (sCT) at 50 μg/animal/day and PTS893 at 5 μg/animal/day to elucidate the mechanisms of action mediating their effects as well as the identification of biomarkers of therapeutic indications. This EXAMPLE is believed to be the first analysis that globally describes the molecular mechanisms of action of salmon calcitonin and a parathyroid hormone analogue by multi-organ-gene-profiling analysis in primates. This is also believed to be the first gene profiling analysis which describes the molecular mechanisms of action of hormonal-mediated bone remodelling by salmon calcitonin and PTS893.

In this EXAMPLE, salmon calcitonin and PTS893 were both found to have modulating effects on genes affecting the direct, autocrine, paracrine and endocrine regulation of the mesenchymal cell functions such as transforming growth factor betas (TGF-βs), insulin-like growth factors (IGFs), bone morphogenetic proteins (BMPs) and vascular endothelial growth factor (VEGF). Both compounds also regulate the synthesis and degradation of extracellular matrix components. Salmon calcitonin also regulates oestrogen receptor and steroidogenic factor, whereas PTS893 produced a strong up-regulation on nuclear receptors of the steroid/thyroid receptor family. These data therefore support the role of calcitonin as an anabolic agent.

In addition, salmon calcitonin and PTS893 also influenced some aspects of the mineralization of the extracellular matrix, since changes in amelogenin, dentin and ectonucleotide pyrophosphatases were observed.

In addition, PTS893 showed an effect on mediating the paracrine activation of osteoclast differentiation and activity, through cytokine and RANK ligand.

No significant differences in gene expression profiling were attributable to the fact of administering salmon calcitonin and PTS893 in combination, with respect to the single therapy.

Thus, gene profiling analysis in this EXAMPLE allowed the reconstruction of the pathways involved in calcitonin and parathyroid hormone signal transduction, triggered by protein-G-linked-receptor stimulation and their influence on cell cycle, as indicated by the changes observed in cyclins.

Animals. A two-week subcutaneous treatment was carried out with salmon calcitonin (sCT), PTS893 or a combination of the two, each of which were dissolved in phosphate buffered saline (PBS) containing 9% autologous serum. Solvent was used as vehicle for the control group.

The animals used in this analysis were cynomolgus monkeys (Macaca fascicularis), supplied by Centre de Recherches Primatologiques, Port Louis, Mauritius. Two animals were used per group and sex. At the beginning of the treatment period, the animals were at least 24 months old, with a body weight of approximately 3 kg. Animals were kept under standard conditions for animal welfare. Animals were examined daily for mortality, food consumption and clinical observations. Body weight was recorded once per week. The dosages were 0 μg/animal/day (as the control), 50 μg/animal/day of salmon calcitonin and 5 μg/animal/day of PTS893.

As shown below, clinical observations and analysis, as well as the histopathological examinations performed in this EXAMPLE, showed that salmon calcitonin administered subcutaneously at a dose of 50 μg/animal/day was well tolerated by the cynomolgus monkeys.

In vivo examinations. No significant histopathological changes were observed. No relevant changes were observed other than a body weight decrease ranging from 8 to 12% in the salmon calcitonin group. A decrease in food consumption was also observed, although not always consistent with the decrease in body weight.

TABLE 1
Food Consumption - Males
Control

| | Day | | | | | | | | | | |
|---|---|---|---|---|---|---|---|---|---|---|---|
| | −6 | −5 | −4 | −3 | −2 | −1 | 1 | 2 | 3 | 4 | 5 |
| Animal no. W62501 | 50 | 100 | 100 | 100 | 100 | 100 | 100 | 100 | 100 | 100 | 100 |
| Animal no. W62502 | 50 | 100 | 100 | 100 | 100 | 100 | 100 | 100 | 100 | 100 | 25 |

| | Day | | | | | | | | | |
|---|---|---|---|---|---|---|---|---|---|---|
| | 6 | 7 | 8 | 9 | 10 | 11 | 12 | 13 | 14 | Avg. |
| Animal no. W62501 | 75 | 100 | 100 | 100 | 100 | 100 | 100 | 25 | | 91.7 |
| Animal no. W62502 | 100 | 75 | 75 | 100 | 100 | 100 | 75 | 100 | 50 | 91.7 |
| Both animals | | | | | | | | | | 91.7 |

Salmon Calcitonin

| | Day | | | | | | | | | | |
|---|---|---|---|---|---|---|---|---|---|---|---|
| | −6 | −5 | −4 | −3 | −2 | −1 | 1 | 2 | 3 | 4 | 5 |
| Animal no. W62503 | 50 | 75 | 50 | 75 | 100 | 75 | 75 | 25 | 50 | 100 | 100 |
| Animal no. W62504 | 50 | 75 | 75 | 75 | 100 | 75 | 50 | 25 | 100 | 75 | 100 |

| | Day | | | | | | | | | |
|---|---|---|---|---|---|---|---|---|---|---|
| | 6 | 7 | 8 | 9 | 10 | 11 | 12 | 13 | 14 | Avg. |
| Animal no. W62503 | 75 | 100 | 100 | 100 | 100 | 75 | 75 | 25 | | 70.8 |
| Animal no. W62504 | 75 | 75 | 75 | 100 | 100 | 75 | 75 | 25 | 75 | 75.0 |
| Both animals | | | | | | | | | | 72.9 |

PTS893

| | Day | | | | | | | | | | |
|---|---|---|---|---|---|---|---|---|---|---|---|
| | −6 | −5 | −4 | −3 | −2 | −1 | 1 | 2 | 3 | 4 | 5 |
| Animal no. W62505 | 50 | 100 | 100 | 100 | 75 | 100 | 100 | 100 | 100 | 100 | 100 |
| Animal no. W62506 | 50 | 100 | 100 | 100 | 100 | 100 | 100 | 100 | 100 | 100 | 100 |

| | Day | | | | | | | | | |
|---|---|---|---|---|---|---|---|---|---|---|
| | 6 | 7 | 8 | 9 | 10 | 11 | 12 | 13 | 14 | Avg. |
| Animal no. W62505 | 100 | 100 | 100 | 100 | 100 | 100 | 100 | 50 | 75 | 87.5 |
| Animal no. W62506 | 100 | 100 | 100 | 100 | 100 | 100 | 100 | 100 | 100 | 91.7 |
| Both animals | | | | | | | | | | 89.6 |

The animals to whom salmon calcitonin was administered presented with a decrease in body weight ranging between 8 to 12%, which can be attributed to a decrease in food consumption. An anorectic effect had previously been described for salmon calcitonin acting through amylin receptors Eiden S et al., J. Physiol. 541(pt3): 1041-1048 (2002); Lutz T A et al., Peptides 21 (2): 233-8 (2000). However, no signs of toxicity were observed here. Hormonal and lipid changes observed in this EXAMPLE are most probably related to a consequent metabolic adaptation.

No relevant changes in electrocardiograms (ECG) or blood pressure were observed.

TABLE 2
Blood Pressure

| Animal | | Compound | Week-1 | Week 2 | Difference |
|---|---|---|---|---|---|
| number | Sex | administered | (mmHg) | (mmHg) | (mmHg) |
| W62501 | Male | Control | 121 | 98 | −23 |
| W62501 | Male | Control | 90 | 29 | −61 |
| W62502 | Male | Control | 86 | 107 | 21 |
| W62502 | Male | Control | 26 | 34 | 8 |
| W62503 | Male | Salmon | 135 | 99 | −36 |
| | | Calcitonin | | | |
| W62503 | Male | Salmon | 61 | 40 | −21 |
| | | Calcitonin | | | |
| W62504 | Male | Salmon | 102 | 79 | −23 |
| | | Calcitonin | | | |
| W62504 | Male | Salmon | 56 | 35 | −21 |
| | | Calcitonin | | | |
| W62505 | Male | PTS893 | 76 | 87 | 11 |
| W62505 | Male | PTS893 | 18 | 22 | 4 |
| W62506 | Male | PTS893 | 106 | 101 | −5 |
| W62506 | Male | PTS893 | 53 | 33 | −20 |
| W62551 | Female | Control | 96 | 76 | −20 |
| W62551 | Female | Control | 27 | 26 | −1 |
| W62552 | Female | Control | 102 | 93 | −9 |
| W62552 | Female | Control | 26 | 36 | 10 |
| W62553 | Female | Salmon | 98 | 82 | −16 |
| | | Calcitonin | | | |
| W62553 | Female | Salmon | 50 | 25 | −25 |
| | | Calcitonin | | | |
| W62554 | Female | Salmon | 92 | 44 | −48 |
| | | Calcitonin | | | |
| W62554 | Female | Salmon | 26 | 30 | 4 |
| | | Calcitonin | | | |
| W62555 | Female | PTS893 | 92 | 70 | −22 |
| W62555 | Female | PTS893 | 43 | 42 | −1 |
| W62556 | Female | PTS893 | 78 | 87 | 9 |
| W62556 | Female | PTS893 | 24 | 28 | 4 |

Blood sampling. Animals were fasted overnight before blood collection but had free access to water. Blood samples were taken from a peripheral vein. Standard haematology and clinical chemistry analysis were performed once during pretest and at the end of the treatment period. Blood samples were collected from each animal at the same intervals as described for the clinical chemistry investigations. The serum samples were deep-frozen (approximately −80° C.) until analyses for hormone determination.

Clinical chemistry and hormone determinations. A slight anaemia was observed in all animals of the study, including the controls. This was attributed to the repeated blood sampling and not considered to be relevant.

TABLE 3
Haematology - Males
Control

| Animal no. | | W62501 | | | W62502 | | |
|---|---|---|---|---|---|---|---|
| Test | Units | d −6 | d 7 | d 13 | d −6 | d 7 | d 13 |
| WBC | G/l | 10.0 | 11.1 | 12.9 | 6.1 | 11.2 | 6.3 |
| RBC | T/l | 7.3 | 6.5 | 6.4 | 6.8 | 6.5 | 6.2 |
| HB | g/dl | 12.9 | 11.9 | 11.7 | 13.1 | 12.3 | 11.9 |
| PCV | l/l | 0.44 | 0.40 | 0.44 | 0.42 | 0.41 | 0.41 |
| MCV | fl | 60 | 61 | 68 | 61 | 63 | 66 |
| MCH | pg | 17.8 | 18.2 | 18.1 | 19.3 | 19.0 | 19.0 |
| MCHC | g/dl | 29.8 | 29.6 | 26.8 | 31.5 | 30.1 | 28.9 |
| PLAT | G/l | 316 | 371 | 266 | 458 | 500 | 547 |
| N | G/l | 6.46 | 4.93 | 3.65 | 2.09 | 6.77 | 1.24 |
| E | G/l | 0.01 | 0.14 | 0.20 | 0.10 | 0.10 | 0.10 |
| B | G/l | 0.02 | 0.03 | 0.06 | 0.02 | 0.02 | 0.00 |
| L | G/l | 3.05 | 5.45 | 8.44 | 3.60 | 3.65 | 4.51 |
| M | G/l | 0.46 | 0.51 | 0.54 | 0.33 | 0.64 | 0.46 |

Salmon Calcitonin

| Animal no. | | W62503 | | | W62504 | | |
|---|---|---|---|---|---|---|---|
| Test | Units | d −6 | d 7 | d 13 | d −6 | d 7 | d 13 |
| WBC | G/l | 7.7 | 11.8 | 8.0 | 11.5 | 9.5 | 8.8 |
| RBC | T/l | 6.3 | 5.9 | 5.6 | 6.9 | 6.0 | 5.4 |
| HB | g/dl | 12.6 | 11.7 | 11.2 | 13.6 | 11.5 | 10.3 |
| PCV | l/l | 0.40 | 0.39 | 0.39 | 0.43 | 0.37 | 0.36 |
| MCV | fl | 64 | 66 | 70 | 62 | 62 | 67 |
| MCH | pg | 20.2 | 19.9 | 20.2 | 19.7 | 19.2 | 19.2 |
| MCHC | g/dl | 31.4 | 30.3 | 29.0 | 32.0 | 31.3 | 28.7 |
| PLAT | G/l | 351 | 396 | 302 | 247 | 330 | 389 |
| N | G/l | 3.36 | 4.11 | 1.90 | 3.93 | 3.31 | 3.04 |
| E | G/l | 0.02 | 0.10 | 0.13 | 0.16 | 0.09 | 0.01 |
| B | G/l | 0.02 | 0.04 | 0.03 | 0.08 | 0.04 | 0.03 |
| L | G/l | 4.00 | 6.79 | 5.38 | 6.55 | 5.57 | 4.92 |
| M | G/l | 0.30 | 0.73 | 0.57 | 0.76 | 0.45 | 0.76 |

PTS893

| Animal no. | | W62505 | | | W62506 | | |
|---|---|---|---|---|---|---|---|
| Test | Units | d −6 | d 7 | d 13 | d −6 | d 7 | d 13 |
| WBC | G/l | 10.4 | 8.4 | 8.8 | 9.1 | 15.0 | 11.9 |
| RBC | T/l | 7.6 | 6.4 | 6.8 | 6.5 | 5.9 | 5.8 |
| HB | g/dl | 13.6 | 11.3 | 11.7 | 13.2 | 11.9 | 11.8 |
| PCV | l/l | 0.43 | 0.38 | 0.43 | 0.40 | 0.40 | 0.41 |
| MCV | fl | 57 | 60 | 63 | 62 | 67 | 70 |
| MCH | pg | 18.0 | 17.7 | 17.3 | 20.4 | 20.2 | 20.3 |
| MCHC | g/dl | 31.5 | 29.3 | 27.5 | 33.1 | 30.2 | 29.2 |
| PLAT | G/l | 325 | 456 | 330 | 459 | 589 | 452 |
| N | G/l | 4.45 | 1.77 | 2.88 | 4.80 | 8.73 | 6.51 |
| E | G/l | 0.21 | 0.30 | 0.19 | 0.03 | 0.08 | 0.07 |
| B | G/l | 0.00 | 0.02 | 0.04 | 0.02 | 0.03 | 0.03 |
| L | G/l | 5.07 | 5.91 | 5.37 | 3.99 | 5.30 | 4.86 |
| M | G/l | 0.62 | 0.39 | 0.27 | 0.27 | 0.83 | 0.46 |

d −6, d 7 and d 13 indicate day −6, day 7 and day 13 relative to the starting day of dosing

TABLE 4
Haematology - Females
Control

| Animal no. | | W62551 | | | W62552 | | |
|---|---|---|---|---|---|---|---|
| Test | Units | d −8 | d 7 | d 13 | d −8 | d 7 | d 13 |
| WBC | pg/ml | 8.2 | 13.7 | 10.0 | 10.1 | 9.1 | 10.4 |
| RBC | nmol/ | 6.5 | 6.2 | 5.8 | 6.7 | 6.2 | 5.8 |
| | l | | | | | | |
| HB | pg/ml | 12.8 | 11.8 | 11.3 | 13.1 | 11.7 | 11.4 |
| PCV | mU/l | 0.42 | 0.43 | 0.41 | 0.42 | 0.42 | 0.41 |
| MCV | pg/ml | 64 | 69 | 71 | 63 | 68 | 70 |
| MCH | ng/ml | 19.7 | 19.1 | 19.4 | 19.5 | 18.9 | 19.5 |
| MCHC | pg/ml | 30.6 | 27.7 | 27.4 | 30.9 | 27.7 | 27.9 |
| PLAT | nmol/ | 463 | 445 | 468 | 286 | 292 | 275 |
| | l | | | | | | |
| N | nmol/ | 4.45 | 5.86 | 3.53 | 6.69 | 3.13 | 4.23 |
| | l | | | | | | |
| E | mUI/l | 0.03 | 0.13 | 0.12 | 0.01 | 0.15 | 0.19 |
| B | pg/ml | 0.03 | 0.07 | 0.04 | 0.02 | 0.03 | 0.03 |
| L | pg/ml | 3.40 | 7.09 | 5.91 | 3.14 | 5.39 | 5.34 |
| M | nmol/ | 0.27 | 0.51 | 0.39 | 0.25 | 0.39 | 0.59 |
| | l | | | | | | |

Salmon Calcitonin

| Animal no. | | W62553 | | | W62554 | | |
|---|---|---|---|---|---|---|---|
| Test | Units | d −8 | d 7 | d 13 | d −8 | d 7 | d 13 |
| WBC | pg/ml | 7.0 | 9.5 | 12.0 | 8.3 | 17.0 | 13.3 |
| RBC | nmol/ | 6.5 | 6.2 | 5.2 | 7.0 | 6.6 | 5.7 |
| | l | | | | | | |
| HB | pg/ml | 12.3 | 11.5 | 10.1 | 13.8 | 12.7 | 11.0 |
| PCV | mU/l | 0.40 | 0.40 | 0.33 | 0.45 | 0.44 | 0.37 |
| MCV | pg/ml | 61 | 64 | 64 | 65 | 68 | 65 |
| MCH | ng/ml | 19.1 | 18.6 | 19.5 | 19.8 | 19.4 | 19.5 |
| MCHC | pg/ml | 31.2 | 29.0 | 30.3 | 30.6 | 28.7 | 29.9 |
| PLAT | nmol/ | 549 | 594 | 451 | 304 | 356 | 229 |
| | l | | | | | | |
| N | nmol/ | 3.45 | 3.83 | 5.41 | 3.13 | 9.82 | 6.16 |
| | l | | | | | | |
| E | mUI/l | 0.03 | 0.36 | 0.73 | 0.03 | 0.04 | 0.06 |
| B | pg/ml | 0.02 | 0.03 | 0.03 | 0.01 | 0.07 | 0.05 |
| L | pg/ml | 3.26 | 4.61 | 5.18 | 4.79 | 6.21 | 6.58 |
| M | nmol/ | 0.25 | 0.63 | 0.69 | 0.30 | 0.82 | 0.39 |
| | l | | | | | | |

PTS893

| Animal no. | | W62555 | | | W62556 | | |
|---|---|---|---|---|---|---|---|
| Test | Units | d −8 | d 7 | d 13 | d −8 | d 7 | d 13 |
| WBC | pg/ml | 10.1 | 18.4 | 13.2 | 14.3 | 12.3 | 10.1 |
| RBC | nmol/ | 6.9 | 6.2 | 5.9 | 6.7 | 6.4 | 5.9 |
| | l | | | | | | |
| HB | pg/ml | 13.4 | 11.7 | 11.3 | 12.9 | 12.1 | 11.3 |
| PCV | mU/l | 0.44 | 0.41 | 0.40 | 0.43 | 0.43 | 0.39 |
| MCV | pg/ml | 63 | 67 | 67 | 64 | 68 | 66 |
| MCH | ng/ml | 19.3 | 18.9 | 19.3 | 19.3 | 19.0 | 19.2 |
| MCHC | pg/ml | 30.6 | 28.2 | 28.6 | 30.2 | 28.1 | 29.2 |
| PLAT | nmol/ | 501 | 525 | 496 | 213 | 382 | 309 |
| | l | | | | | | |
| N | nmol/ | 5.34 | 10.8 | 6.36 | 9.05 | 5.49 | 4.18 |
| | l | | | | | | |
| E | mUI/l | 0.00 | 0.12 | 0.21 | 0.26 | 0.49 | 0.29 |
| B | pg/ml | 0.00 | 0.06 | 0.03 | 0.03 | 0.04 | 0.04 |
| L | pg/ml | 3.92 | 6.29 | 5.81 | 4.40 | 5.87 | 5.21 |
| M | nmol/ | 0.80 | 1.12 | 0.82 | 0.54 | 0.44 | 0.37 |
| | l | | | | | | |

d −8, d 7 and d 13 indicate day −8, day 7 and day 13 relative to the starting day of dosing

Among the standard clinical chemistry tests performed, slight to moderate decreases in phosphorus and/or magnesium and a moderate to marked decrease in triglycerides were seen in the groups administered salmon calcitonin and PTS893.

TABLE 5
Clinical Chemistry - Males
Control

| Animal no. | | W62501 | | | W62502 | | |
|---|---|---|---|---|---|---|---|
| Test | Units | d −6 | d 7 | d 13 | d −6 | d 7 | d 13 |
| Na+ | mmol/l | 154 | 151 | 153 | 152 | 153 | 148 |
| K+ | mmol/l | 4.05 | 5.31 | 4.26 | 4.09 | 4.05 | 4.51 |
| Cl− | mmol/l | 109 | 113 | 108 | 107 | 110 | 111 |
| Ca++ | mmol/l | 2.57 | 2.47 | 2.69 | 2.72 | 2.52 | 2.75 |
| I.PHOS | mmol/l | 2.21 | 1.93 | 2.76 | 1.88 | 1.69 | 1.99 |
| Mg++ | mmol/l | 1.09 | 0.91 | 0.95 | 0.88 | 0.79 | 1.14 |
| GLUC | mmol/l | 3.85 | 4.51 | 4.68 | 3.44 | 5.30 | 6.13 |
| UREA | mmol/l | 9.7 | 4.9 | 5.0 | 7.6 | 6.1 | 5.2 |
| CREAT | μmol/l | 85 | 60 | 75 | 65 | 55 | 57 |
| TOT.BIL. | μmol/l | 6.0 | 2.0 | 2.0 | 7.0 | 3.0 | 4.0 |
| PROT | g/l | 89 | 80 | 88 | 90 | 83 | 85 |
| A/G | | 1.89 | 1.57 | 1.45 | 1.62 | 1.53 | 1.50 |
| CHOL | mmol/l | 3.30 | 3.20 | 3.50 | 3.30 | 3.40 | 3.10 |
| HDL-CHOL | mmol/l | 1.49 | 1.45 | 1.70 | 1.54 | 1.45 | 1.49 |
| LDL-CHOL | mmol/l | 1.63 | 1.62 | 1.84 | 1.56 | 1.93 | 1.49 |
| TRIG | mmol/l | 0.94 | 0.36 | 0.43 | 0.65 | 0.36 | 0.45 |
| ALP | IU/l | 1559 | 1241 | 1313 | 1463 | 1423 | 1493 |
| BAP-E | IU/l | 543 | 439 | 457 | 452 | 476 | 464 |
| ASAT | IU/l | 22 | 22 | 25 | 30 | 26 | 26 |
| ALAT | IU/l | 22 | 32 | 30 | 29 | 41 | 37 |
| CK | IU/l | 150 | 45 | 127 | 74 | 67 | 102 |
| LDH | IU/l | 392 | 585 | 549 | 421 | 518 | 592 |
| GGT | IU/l | 128 | 92 | 111 | 89 | 71 | 75 |
| ALB | % | 65 | 61 | 59 | 62 | 61 | 60 |
| A1-GLOB | % | 1.90 | 2.70 | 2.50 | 1.90 | 2.10 | 2.30 |
| A2-GLOB | % | 7.60 | 8.30 | 7.90 | 8.20 | 8.90 | 8.50 |
| B-GLOB | % | 16 | 18 | 19 | 18 | 19 | 19 |
| G-GLOB | % | 9.2 | 9.9 | 10.9 | 9.6 | 9.3 | 10.2 |
| ALB | g/l | 58 | 49 | 52 | 56 | 50 | 51 |
| A1-GLOB | g/l | 1.70 | 2.20 | 2.20 | 1.70 | 1.70 | 2.00 |
| A2-GLOB | g/l | 6.80 | 6.60 | 7.00 | 7.40 | 7.40 | 7.20 |
| B-GLOB | g/l | 14 | 14 | 17 | 17 | 16 | 16 |
| G-GLOB | g/l | 8.2 | 7.9 | 9.6 | 8.6 | 7.7 | 8.7 |

Salmon Calcitonin

| Animal no. | | W62503 | | | W62504 | | |
|---|---|---|---|---|---|---|---|
| Test | Units | d −6 | d 7 | d 13 | d −6 | d 7 | d 13 |
| Na+ | mmol/l | 151 | 145 | 148 | 154 | 142 | 144 |
| K+ | mmol/l | 4.24 | 4.90 | 4.34 | 4.85 | 5.15 | 4.48 |
| Cl− | mmol/l | 107 | 104 | 104 | 113 | 106 | 101 |
| Ca++ | mmol/l | 2.66 | 2.68 | 2.91 | 2.71 | 2.54 | 2.73 |
| I.PHOS | mmol/l | 2.05 | 1.67 | 2.06 | 2.10 | 1.73 | 1.94 |
| Mg++ | mmol/l | 0.97 | 0.68 | 0.73 | 0.99 | 0.71 | 0.72 |
| GLUC | mmol/l | 3.57 | 3.58 | 4.29 | 3.70 | 4.98 | 6.19 |
| UREA | mmol/l | 7.9 | 1.3 | 2.9 | 6.6 | 3.3 | 2.9 |
| CREAT | μmol/l | 78 | 57 | 62 | 64 | 50 | 56 |
| TOT.BIL. | μmol/l | 5.0 | 2.0 | 1.0 | 3.0 | 2.0 | 2.0 |
| PROT | g/l | 87 | 82 | 87 | 91 | 83 | 89 |
| A/G | | 1.76 | 1.68 | 1.42 | 1.42 | 1.26 | 1.05 |
| CHOL | mmol/l | 3.30 | 3.60 | 3.70 | 3.80 | 3.90 | 3.40 |
| HDL-CHOL | mmol/l | 1.49 | 2.09 | 2.44 | 1.46 | 1.48 | 1.39 |
| LDL-CHOL | mmol/l | 1.21 | 1.28 | 1.26 | 1.87 | 2.51 | 1.83 |
| TRIG | mmol/l | 0.96 | 0.24 | 0.27 | 0.92 | 0.22 | 0.68 |
| ALP | IU/l | 1488 | 1023 | 1226 | 857 | 587 | 626 |
| BAP-E | IU/l | 508 | 363 | 302 | 311 | 188 | 180 |
| ASAT | IU/l | 28 | 31 | 28 | 24 | 17 | 24 |
| ALAT | IU/l | 38 | 39 | 43 | 48 | 24 | 31 |
| CK | IU/l | 124 | 56 | 119 | 75 | 45 | 173 |
| LDH | IU/l | 439 | 400 | 427 | 356 | 384 | 519 |
| GGT | IU/l | 105 | 80 | 75 | 121 | 75 | 69 |
| ALB | % | 64 | 63 | 59 | 59 | 56 | 51 |
| A1-GLOB | % | 1.60 | 2.00 | 2.40 | 1.90 | 2.80 | 3.60 |
| A2-GLOB | % | 8.00 | 8.80 | 8.80 | 8.70 | 8.70 | 7.80 |
| B-GLOB | % | 18 | 18 | 20 | 19 | 21 | 24 |
| G-GLOB | % | 8.3 | 8.5 | 9.7 | 12.0 | 12.1 | 13.6 |
| ALB | g/l | 56 | 51 | 51 | 54 | 46 | 46 |
| A1-GLOB | g/l | 1.40 | 1.60 | 2.10 | 1.70 | 2.30 | 3.20 |
| A2-GLOB | g/l | 7.00 | 7.20 | 7.70 | 7.90 | 7.20 | 6.90 |
| B-GLOB | g/l | 16 | 15 | 18 | 17 | 17 | 21 |
| G-GLOB | g/l | 7.2 | 7.0 | 8.4 | 10.9 | 10.0 | 12.1 |

PTS893

| Animal no. | | W62505 | | | W62506 | | |
|---|---|---|---|---|---|---|---|
| Test | Units | d −6 | d 7 | d 13 | d −6 | d 7 | d 13 |
| Na+ | mmol/l | 151 | 151 | 152 | 151 | 149 | 149 |
| K+ | mmol/l | 5.13 | 4.00 | 4.27 | 4.72 | 4.76 | 4.12 |
| Cl− | mmol/l | 110 | 107 | 110 | 112 | 106 | 106 |
| Ca++ | mmol/l | 2.81 | 2.39 | 2.59 | 2.64 | 2.45 | 2.51 |
| I.PHOS | mmol/l | 2.59 | 1.68 | 2.22 | 2.12 | 1.12 | 1.77 |
| Mg++ | mmol/l | 1.04 | 0.71 | 0.77 | 0.97 | 0.70 | 0.76 |
| GLUC | mmol/l | 5.09 | 4.76 | 5.42 | 3.88 | 5.26 | 4.96 |
| UREA | mmol/l | 11.6 | 3.7 | 6.4 | 15.0 | 4.9 | 5.8 |
| CREAT | μmol/l | 86 | 66 | 79 | 77 | 63 | 70 |
| TOT.BIL. | μmol/l | 5.0 | 2.0 | 1.0 | 7.0 | 2.0 | 1.0 |
| PROT | g/l | 81 | 74 | 81 | 88 | 86 | 89 |
| A/G | | 1.89 | 1.70 | 1.76 | 1.58 | 1.28 | 1.40 |
| CHOL | mmol/l | 3.20 | 3.30 | 3.10 | 2.50 | 2.50 | 2.60 |
| HDL-CHOL | mmol/l | 1.49 | 1.49 | 1.61 | 1.24 | 1.25 | 1.38 |
| LDL-CHOL | mmol/l | 1.39 | 1.73 | 1.51 | 1.27 | 1.22 | 1.38 |
| TRIG | mmol/l | 0.96 | 0.30 | 0.63 | 0.49 | 0.39 | 0.35 |
| ALP | IU/l | 1703 | 1494 | 1768 | 1414 | 1363 | 1486 |
| BAP-E | IU/l | 523 | 532 | 564 | 445 | 423 | 497 |
| ASAT | IU/l | 24 | 18 | 24 | 25 | 27 | 29 |
| ALAT | IU/l | 32 | 30 | 27 | 23 | 19 | 20 |
| CK | IU/l | 111 | 82 | 148 | 86 | 73 | 125 |
| LDH | IU/l | 367 | 400 | 528 | 354 | 432 | 464 |
| GGT | IU/l | 133 | 99 | 105 | 112 | 85 | 91 |
| ALB | % | 66 | 63 | 64 | 61 | 56 | 59 |
| A1-GLOB | % | 2.20 | 2.80 | 2.60 | 2.40 | 3.60 | 2.80 |
| A2-GLOB | % | 8.80 | 8.90 | 8.70 | 7.30 | 8.30 | 7.50 |
| B-GLOB | % | 17 | 18 | 19 | 19 | 22 | 20 |
| G-GLOB | % | 6.9 | 6.9 | 6.3 | 9.8 | 10.5 | 10.9 |
| ALB | g/l | 53 | 47 | 52 | 54 | 48 | 52 |
| A1-GLOB | g/l | 1.80 | 2.10 | 2.10 | 2.10 | 3.10 | 2.50 |
| A2-GLOB | g/l | 7.10 | 6.60 | 7.10 | 6.40 | 7.10 | 6.70 |
| B-GLOB | g/l | 14 | 14 | 15 | 17 | 19 | 18 |
| G-GLOB | g/l | 5.6 | 5.1 | 5.1 | 8.6 | 9.0 | 9.7 |

d −6, d 7 and d 13 indicate day −6, day 7 and day 13 relative to the starting day of dosing

TABLE 6
Clinical Chemistry - Females
Control

| Animal no. | | W62551 | | | W62552 | | |
|---|---|---|---|---|---|---|---|
| Test | Units | d −8 | d 7 | d 13 | d −8 | d 7 | d 13 |
| Na+ | mmol/l | 152 | 148 | 155 | 148 | 150 | 148 |
| K+ | mmol/l | 4.16 | 4.23 | 4.92 | 3.82 | 4.11 | 5.27 |
| Cl− | mmol/l | 110 | 105 | 111 | 109 | 106 | 108 |
| Ca++ | mmol/l | 2.64 | 2.61 | 2.61 | 2.48 | 2.44 | 1.80 |
| I.PHOS | mmol/l | 1.98 | 2.61 | 2.28 | 1.84 | 1.98 | 1.84 |
| Mg++ | mmol/l | 1.00 | 0.97 | 1.03 | 0.88 | 0.84 | 0.31 |
| GLUC | mmol/l | 3.65 | 8.39 | 3.86 | 2.79 | 3.86 | 3.60 |
| UREA | mmol/l | 11.0 | 8.3 | 8.2 | 11.3 | 6.9 | 6.3 |
| CREAT | μmol/l | 73 | 77 | 62 | 67 | 60 | 50 |
| TOT.BIL. | μmol/l | 4.00 | 2.00 | 3.00 | 5.00 | 1.00 | 2.00 |
| PROT | g/l | 85 | 80 | 80 | 83 | 83 | 77 |
| A/G | | 1.77 | 1.67 | 1.55 | 1.68 | 1.39 | 1.27 |
| CHOL | mmol/l | 3.20 | 2.80 | 3.00 | 3.70 | 3.40 | 3.50 |
| HDL-CHOL | mmol/l | 1.63 | 1.44 | 1.49 | 1.75 | 1.82 | 1.80 |
| LDL-CHOL | mmol/l | 1.55 | 1.25 | 1.90 | 1.57 | 1.28 | 1.66 |
| TRIG | mmol/l | 0.64 | 0.54 | 0.57 | 0.83 | 0.48 | 0.50 |
| ALP | IU/l | 1037 | 1088 | 1187 | 1332 | 1298 | 1182 |
| BAP-E | IU/l | 310 | 369 | 346 | 432 | 419 | 379 |
| ASAT | IU/l | 27 | 33 | 31 | 21 | 22 | 23 |
| ALAT | IU/l | 44 | 52 | 46 | 16 | 19 | 20 |
| CK | IU/l | 69 | 169 | 81 | 83 | 68 | 87 |
| LDH | IU/l | 420 | 520 | 481 | 474 | 471 | 516 |
| GGT | IU/l | 104 | 95 | 102 | 84 | 67 | 66 |
| ALB | % | 64 | 63 | 61 | 63 | 58 | 56 |
| A1-GLOB | % | 1.90 | 2.60 | 3.40 | 2.00 | 2.60 | 3.50 |
| A2-GLOB | % | 8.00 | 7.60 | 7.70 | 7.00 | 8.10 | 7.70 |
| B-GLOB | % | 17 | 18 | 18 | 15 | 18 | 18 |
| G-GLOB | % | 9.4 | 9.2 | 9.9 | 12.9 | 13.2 | 14.8 |
| ALB | g/l | 54 | 50 | 49 | 52 | 48 | 43 |
| A1-GLOB | g/l | 1.60 | 2.10 | 2.70 | 1.70 | 2.20 | 2.70 |
| A2-GLOB | g/l | 6.80 | 6.10 | 6.20 | 5.80 | 6.70 | 5.90 |
| B-GLOB | g/l | 14 | 14 | 15 | 13 | 15 | 14 |
| G-GLOB | g/l | 8.0 | 7.4 | 7.9 | 10.7 | 11.0 | 11.4 |

Salmon Calcitonin

| Animal no. | | W62553 | | | W62554 | | |
|---|---|---|---|---|---|---|---|
| Test | Units | d −8 | d 7 | d 13 | d −8 | d 7 | d 13 |
| Na+ | mmol/l | 145 | 147 | 147 | 145 | 143 | 147 |
| K+ | mmol/l | 3.51 | 3.73 | 4.62 | 3.89 | 4.07 | 4.95 |
| Cl− | mmol/l | 106 | 104 | 107 | 100 | 96 | 107 |
| Ca++ | mmol/l | 2.62 | 2.77 | 2.57 | 2.73 | 2.91 | 2.68 |
| I.PHOS | mmol/l | 1.62 | 1.48 | 1.81 | 1.97 | 1.75 | 1.83 |
| Mg++ | mmol/l | 0.87 | 0.63 | 0.76 | 0.91 | 0.77 | 0.80 |
| GLUC | mmol/l | 3.84 | 4.88 | 4.98 | 4.11 | 5.31 | 4.04 |
| UREA | mmol/l | 10.3 | 6.6 | 5.0 | 10.0 | 6.3 | 5.9 |
| CREAT | μmol/l | 81 | 71 | 61 | 88 | 77 | 65 |
| TOT.BIL. | μmol/l | 3.00 | 2.00 | 2.00 | 6.00 | 5.00 | 2.00 |
| PROT | g/l | 88 | 90 | 80 | 91 | 95 | 83 |
| A/G | | 1.46 | 1.45 | 1.30 | 1.48 | 1.42 | 1.26 |
| CHOL | mmol/l | 2.70 | 2.80 | 2.20 | 3.30 | 4.00 | 3.00 |
| HDL-CHOL | mmol/l | 1.04 | 1.11 | 0.96 | 1.46 | 1.99 | 1.66 |
| LDL-CHOL | mmol/l | 1.61 | 1.51 | 1.46 | 1.13 | 1.93 | 1.42 |
| TRIG | mmol/l | 0.79 | 0.25 | 0.39 | 0.88 | 0.30 | 0.38 |
| ALP | IU/l | 1197 | 965 | 842 | 1132 | 877 | 890 |
| BAP-E | IU/l | 416 | 326 | 304 | 344 | 325 | 294 |
| ASAT | IU/l | 24 | 21 | 25 | 20 | 18 | 20 |
| ALAT | IU/l | 21 | 24 | 19 | 19 | 14 | 19 |
| CK | IU/l | 99 | 72 | 107 | 76 | 64 | 77 |
| LDH | IU/l | 286 | 423 | 429 | 319 | 372 | 363 |
| GGT | IU/l | 88 | 63 | 54 | 82 | 72 | 62 |
| ALB | % | 59 | 59 | 57 | 60 | 59 | 56 |
| A1-GLOB | % | 2.70 | 2.70 | 3.10 | 2.20 | 2.20 | 3.10 |
| A2-GLOB | % | 6.50 | 6.10 | 6.80 | 8.00 | 7.70 | 7.80 |
| B-GLOB | % | 21 | 23 | 21 | 15 | 17 | 17 |
| G-GLOB | % | 10.8 | 8.6 | 12.4 | 14.9 | 14.6 | 16.3 |
| ALB | g/l | 52 | 54 | 45 | 54 | 56 | 46 |
| A1-GLOB | g/l | 2.40 | 2.40 | 2.50 | 2.00 | 2.10 | 2.60 |
| A2-GLOB | g/l | 5.70 | 5.50 | 5.40 | 7.30 | 7.30 | 6.50 |
| B-GLOB | g/l | 18 | 21 | 17 | 14 | 16 | 14 |
| G-GLOB | g/l | 9.5 | 7.7 | 9.9 | 13.6 | 13.9 | 13.5 |

PTS893

| Animal no. | | W62555 | | | W62556 | | |
|---|---|---|---|---|---|---|---|
| Test | Units | d −8 | d 7 | d 13 | d −8 | d 7 | d 13 |
| Na+ | mmol/l | 153 | 151 | 152 | 150 | 148 | 149 |
| K+ | mmol/l | 4.82 | 4.54 | 4.63 | 3.85 | 3.81 | 4.31 |
| Cl− | mmol/l | 107 | 109 | 111 | 108 | 107 | 114 |
| Ca++ | mmol/l | 2.77 | 2.61 | 2.20 | 2.64 | 2.62 | 2.35 |
| I.PHOS | mmol/l | 2.11 | 1.31 | 1.51 | 2.10 | 1.60 | 1.50 |
| Mg++ | mmol/l | 0.96 | 0.65 | 0.59 | 0.90 | 0.74 | 0.66 |
| GLUC | mmol/l | 3.57 | 4.18 | 3.59 | 3.22 | 4.45 | 3.52 |
| UREA | mmol/l | 8.2 | 8.7 | 6.3 | 8.4 | 6.6 | 6.8 |
| CREAT | μmol/l | 77 | 62 | 58 | 68 | 63 | 58 |
| TOT.BIL. | μmol/l | 5.00 | 1.00 | 2.00 | 5.00 | 2.00 | 2.00 |
| PROT | g/l | 89 | 87 | 78 | 84 | 83 | 76 |
| A/G | | 1.64 | 1.62 | 1.65 | 1.84 | 1.78 | 1.50 |
| CHOL | mmol/l | 2.90 | 2.70 | 2.80 | 2.70 | 2.40 | 2.70 |
| HDL-CHOL | mmol/l | 1.31 | 1.48 | 1.51 | 1.12 | 0.99 | 1.25 |
| LDL-CHOL | mmol/l | 1.69 | 1.12 | 1.71 | 1.62 | 1.28 | 1.58 |
| TRIG | mmol/l | 0.59 | 0.27 | 0.25 | 0.67 | 0.34 | 0.47 |
| ALP | IU/l | 1535 | 1223 | 1332 | 1638 | 1307 | 1313 |
| BAP-E | IU/l | 457 | 350 | 426 | 456 | 390 | 400 |
| ASAT | IU/l | 23 | 18 | 25 | 24 | 20 | 25 |
| ALAT | IU/l | 35 | 25 | 32 | 33 | 19 | 21 |
| CK | IU/l | 84 | 65 | 175 | 63 | 144 | 172 |
| LDH | IU/l | 468 | 465 | 557 | 309 | 313 | 358 |
| GGT | IU/l | 85 | 71 | 70 | 103 | 85 | 83 |
| ALB | % | 62 | 62 | 62 | 65 | 64 | 60 |
| A1-GLOB | % | 2.30 | 2.50 | 2.50 | 1.90 | 2.10 | 2.70 |
| A2-GLOB | % | 7.50 | 8.00 | 8.30 | 7.50 | 7.50 | 8.10 |
| B-GLOB | % | 18 | 19 | 18 | 17 | 17 | 20 |
| G-GLOB | % | 9.7 | 8.4 | 8.7 | 8.8 | 9.1 | 8.7 |
| ALB | g/l | 55 | 54 | 49 | 55 | 53 | 46 |
| A1-GLOB | g/l | 2.10 | 2.20 | 2.00 | 1.60 | 1.70 | 2.10 |
| A2-GLOB | g/l | 6.70 | 7.00 | 6.50 | 6.30 | 6.20 | 6.20 |
| B-GLOB | g/l | 16 | 17 | 14 | 14 | 14 | 16 |
| G-GLOB | g/l | 8.6 | 7.3 | 6.8 | 7.4 | 7.6 | 6.6 |

d −8, d 7 and d 13 indicate day −8, day 7 and day 13 relative to the starting day of dosing

No relevant changes were observed in the standard urinalysis tests performed.

TABLE 7
Urinary analysis - Males
Control

| Animal no. | | W62501 | | | W62502 | | |
|---|---|---|---|---|---|---|---|
| Test | Units | −6 | −5 | 13 | −6 | −5 | 13 |
| VOLUME | ml | 15 | 10 | 77 | 22 | 130 | 30 |
| CREAT | μmol/l | 18000 | 17000 | 5460 | 7920 | 2480 | 5160 |
| NTx | nM BCE | — | 9954 | 3425 | — | 11979 | 3167 |
| CTx | μg/l | — | 21592 | 6810 | — | 27169 | 5323 |
| D-PYR | nmol/l | — | 2345 | 1110 | — | 2904 | 1461 |
| LDH | IU/L | 6.0 | | nd | 8.0 | | 8.0 |
| NAG | IU/l | 3.5 | | 1.5 | 3.2 | | 1.6 |
| Na+ | mmol/l | 163 | | 43 | 87 | | 77 |
| K+ | mmol/l | 258 | | 67 | 125 | | 75 |
| Cl− | mmol/l | 132 | | 43 | 52 | | 59 |
| Ca2+ | mmol/l | 5.15 | | 16.80 | 15.95 | | 15.50 |
| I.PHOS | mmol/l | 11.10 | | 1.05 | 11.30 | | 8.90 |
| Mg2+ | mmol/l | 2.75 | | 7.50 | 7.85 | | 6.25 |
| Na/Crea | mM/mM | 9.10 | | 7.90 | 11.00 | | 14.90 |
| K/Crea | mM/mM | 14.30 | | 12.20 | 15.80 | | 14.50 |
| Cl/Crea | mM/mM | 7.40 | | 7.90 | 6.50 | | 11.40 |
| Ca/Crea | mM/mM | 0.29 | | 3.08 | 2.01 | | 3.00 |
| Pho/Crea | mM/mM | 0.62 | | 0.19 | 1.43 | | 1.73 |
| Mg/Crea | mM/mM | 0.20 | | 1.40 | 1.00 | | 1.20 |
| LDH/crea | IU/mM | 0.33 | | nd | 1.01 | | 1.55 |
| NAG/crea | IU/mM | 0.19 | | 0.28 | 0.40 | | 0.31 |
| NTx/Crea | nME/mM | | 586 | 627 | | 4830 | 614 |
| CTx/Crea | μg/μm. | | 1270 | 1247 | | 10955 | 1032 |
| Pyr/Crea | nM/mM | | 138 | 203 | | 1171 | 283 |

Salmon Calcitonin

| Animal no. | | W62503 | | | W62504 | | |
|---|---|---|---|---|---|---|---|
| Test | Units | −6 | −5 | 13 | −6 | −5 | 13 |
| VOLUME | ml | 62 | 38 | 68 | 37 | 10 | 54 |
| CREAT | μmol/l | 4300 | 7840 | 4620 | 13600 | 17360 | 4400 |
| NTx | nM BCE | — | 6023 | 5186 | — | 16067 | 3790 |
| CTx | μg/l | — | 11618 | 10088 | — | 26370 | 6130 |
| D-PYR | nmol/l | — | 1733 | 1083 | — | 5113 | 1476 |
| LDH | IU/L | 9.0 | | 7.0 | 13.0 | | 17.0 |
| NAG | IU/l | 2.7 | | 1.4 | 4.2 | | 7.2 |
| Na+ | mmol/l | 22 | | 14 | 119 | | 15 |
| K+ | mmol/l | 65 | | 78 | 134 | | 76 |
| Cl− | mmol/l | 10 | | 55 | 64 | | 68 |
| Ca2+ | mmol/l | 0.90 | | 18.25 | 3.70 | | 23.40 |
| I.PHOS | mmol/l | 4.35 | | 2.50 | 5.33 | | 3.00 |
| Mg2+ | mmol/l | 1.40 | | 7.05 | 7.55 | | 9.80 |
| Na/Crea | mM/mM | 5.20 | | 3.10 | 8.70 | | 3.40 |
| K/Crea | mM/mM | 15.10 | | 16.90 | 9.90 | | 17.20 |
| Cl/Crea | mM/mM | 2.20 | | 11.80 | 4.70 | | 15.30 |
| Ca/Crea | mM/mM | 0.21 | | 3.95 | 0.27 | | 5.32 |
| Pho/Crea | mM/mM | 1.01 | | 0.54 | 0.39 | | 0.68 |
| Mg/Crea | mM/mM | 0.30 | | 1.50 | 0.60 | | 2.20 |
| LDH/crea | IU/mM | 2.09 | | 1.52 | 0.96 | | 3.86 |
| NAG/crea | IU/mM | 0.63 | | 0.30 | 0.31 | | 1.64 |
| NTx/Crea | nME/mM | | 768 | 1123 | | 926 | 861 |
| CTx/Crea | μg/μm. | | 1482 | 2184 | | 1519 | 1393 |
| Pyr/Crea | nM/mM | | 221 | 234 | | 295 | 336 |

PTS893

| Animal no. | | W62505 | | | W62506 | | |
|---|---|---|---|---|---|---|---|
| Test | Units | −6 | −5 | 13 | −6 | −5 | 13 |
| VOLUME | ml | 14 | 14 | 48 | 58 | 34 | 130 |
| CREAT | μmol/l | 16160 | 16160 | 7840 | 9940 | 16120 | 3840 |
| NTx | nM BCE | — | 5403 | 4871 | — | 8757 | 2102 |
| CTx | μg/l | — | 11865 | 9365 | — | 20108 | 3705 |
| D-PYR | nmol/l | — | 1660 | 1676 | — | 2278 | 782 |
| LDH | IU/L | 7.0 | | 14.0 | 9.0 | | 19.0 |
| NAG | IU/l | 23.4 | | 2.9 | 7.1 | | 2.6 |
| Na+ | mmol/l | 174 | | 111 | 59 | | 35 |
| K+ | mmol/l | 86 | | 107 | 125 | | 69 |
| Cl− | mmol/l | 22 | | 117 | 50 | | 48 |
| Ca2+ | mmol/l | 5.10 | | 7.55 | 3.50 | | 13.10 |
| I.PHOS | mmol/l | 74.40 | | 0.10 | 3.86 | | 0.17 |
| Mg2+ | mmol/l | 11.25 | | 8.70 | 2.95 | | 5.25 |
| Na/Crea | mM/mM | 10.80 | | 14.10 | 6.00 | | 9.10 |
| K/Crea | mM/mM | 5.30 | | 13.60 | 12.60 | | 17.90 |
| Cl/Crea | mM/mM | 1.40 | | 15.00 | 5.00 | | 12.60 |
| Ca/Crea | mM/mM | 0.32 | | 0.96 | 0.35 | | 3.41 |
| Pho/Crea | mM/mM | 4.60 | | 0.01 | 0.39 | | 0.04 |
| Mg/Crea | mM/mM | 0.70 | | 1.10 | 0.30 | | 1.40 |
| LDH/crea | IU/mM | 0.43 | | 1.79 | 0.91 | | 4.95 |
| NAG/crea | IU/mM | 1.45 | | 0.37 | 0.71 | | 0.68 |
| NTx/Crea | nME/mM | | 334 | 621 | | 543 | 547 |
| CTx/Crea | μg/μm. | | 734 | 1195 | | 1247 | 965 |
| Pyr/Crea | nM/mM | | 103 | 214 | | 141 | 204 |

d −6, d −5 and d 13 indicate day −6, day −5 and day 13 relative to the starting day of dosing

TABLE 8
Urinary analysis - Females
Control

| Animal no. | | W62551 | | | W62552 | | |
|---|---|---|---|---|---|---|---|
| Test | Units | −8 | −7 | 13 | −8 | −7 | 13 |
| VOLUME | ml | 21 | 21 | 43 | 18 | 53 | 53 |
| CREAT | μmol/l | 16420 | 16420 | 9560 | 14300 | 6700 | 5380 |
| NTx | nM BCE | — | 9248 | 7824 | — | 5053 | 4695 |
| CTx | μg/l | — | 19280 | 17916 | — | 12014 | 10557 |
| D-PYR | nmol/l | — | 2500 | 2748 | — | 1397 | 2159 |
| LDH | IU/L | 10.0 | | 15.0 | 9.0 | | 25.0 |
| NAG | IU/l | 19.2 | | 4.2 | 10.3 | | 3.5 |
| Na+ | mmol/l | 110 | | 44 | 140 | | 64 |
| K+ | mmol/l | 82 | | 122 | 124 | | 87 |
| Cl− | mmol/l | 24 | | 73 | 72 | | 56 |
| Ca2+ | mmol/l | 2.90 | | 16.10 | 11.90 | | 19.50 |
| I.PHOS | mmol/l | 88.2 | | 7.7 | 20.3 | | 3.5 |
| Mg2+ | mmol/l | 2.35 | | 7.20 | 9.00 | | 5.45 |
| Na/Crea | mM/mM | 6.70 | | 4.60 | 9.80 | | 11.90 |
| K/Crea | mM/mM | 5.00 | | 12.80 | 8.70 | | 16.20 |
| Cl/Crea | mM/mM | 1.50 | | 7.60 | 5.10 | | 10.50 |
| Ca/Crea | mM/mM | 0.18 | | 1.68 | 0.83 | | 3.63 |
| Pho/Crea | mM/mM | 5.37 | | 0.81 | 1.42 | | 0.64 |
| Mg/Crea | mM/mM | 0.10 | | 0.80 | 0.60 | | 1.00 |
| LDH/crea | IU/mM | 0.61 | | 1.57 | 0.63 | | 4.65 |
| NAG/crea | IU/mM | 1.17 | | 0.44 | 0.72 | | 0.65 |
| NTx/Crea | nME/mM | | 563 | 818 | | 754 | 873 |
| CTx/Crea | μg/μm. | | 1174 | 1874 | | 1793 | 1962 |
| Pyr/Crea | nM/mM | | 152 | 288 | | 209 | 401 |

Salmon Calcitonin

| Animal no. | | W62553 | | | W62554 | | |
|---|---|---|---|---|---|---|---|
| Test | Units | −8 | −7 | 13 | −8 | −7 | 13 |
| VOLUME | ml | 11 | 58 | 67 | 32 | 14 | 49 |
| CREAT | μmol/l | 10780 | 6920 | 4800 | 11260 | 13380 | 4200 |
| NTx | nM BCE | — | 4624 | 3465 | — | 7393 | 2812 |
| CTx | μg/l | — | 6983 | 5392 | — | 13411 | 5631 |
| D-PYR | nmol/l | — | 2762 | 1644 | — | 2016 | 1110 |
| LDH | IU/L | 14.0 | | 6.0 | 6.0 | | 36.0 |
| NAG | IU/l | 10.2 | | 2.8 | 1.2 | | 2.7 |
| Na+ | mmol/l | 98 | | 40 | 156 | | 32 |
| K+ | mmol/l | 104 | | 53 | 172 | | 57 |
| Cl− | mmol/l | 31 | | 63 | 156 | | 65 |
| Ca2+ | mmol/l | 3.00 | | 17.55 | 3.50 | | 12.70 |
| I.PHOS | mmol/l | 25.4 | | 5.1 | 10.8 | | 5.8 |
| Mg2+ | mmol/l | 3.35 | | 5.40 | 3.80 | | 4.85 |
| Na/Crea | mM/mM | 9.10 | | 8.30 | 13.90 | | 7.60 |
| K/Crea | mM/mM | 9.60 | | 11.10 | 15.20 | | 13.50 |
| Cl/Crea | mM/mM | 2.90 | | 13.20 | 13.80 | | 15.40 |
| Ca/Crea | mM/mM | 0.28 | | 3.66 | 0.31 | | 3.02 |
| Pho/Crea | mM/mM | 2.35 | | 1.05 | 0.96 | | 1.38 |
| Mg/Crea | mM/mM | 0.30 | | 1.10 | 0.30 | | 1.20 |
| LDH/crea | IU/mM | 1.30 | | 1.25 | 0.53 | | 8.57 |
| NAG/crea | IU/mM | 0.95 | | 0.58 | 0.11 | | 0.64 |
| NTx/Crea | nME/mM | | 668 | 722 | | 553 | 670 |
| CTx/Crea | μg/μm. | | 1009 | 1123 | | 1002 | 1341 |
| Pyr/Crea | nM/mM | | 399 | 343 | | 151 | 264 |

PTS893

| Animal no. | | W62555 | | | W62556 | | |
|---|---|---|---|---|---|---|---|
| Test | Units | −8 | −7 | 13 | −8 | −7 | 13 |
| VOLUME | ml | 14 | 15 | 52 | 39 | 69 | 42 |
| CREAT | μmol/l | 19160 | 18240 | 5620 | 14060 | 7600 | 8060 |
| NTx | nM BCE | — | 10499 | 2514 | — | 4818 | 5679 |
| CTx | μg/l | — | 21919 | 3813 | — | 8877 | 11236 |
| D-PYR | nmol/l | — | 2963 | 1356 | — | 1377 | 2036 |
| LDH | IU/L | 11.0 | | 10.0 | 18.0 | | 9.0 |
| NAG | IU/l | 0.5 | | 1.2 | 5.9 | | 5.1 |
| Na+ | mmol/l | 145 | | 71 | 118 | | 146 |
| K+ | mmol/l | 302 | | 150 | 164 | | 70 |
| Cl− | mmol/l | 119 | | 101 | 53 | | 133 |
| Ca2+ | mmol/l | 11.50 | | 20.05 | 6.60 | | 12.35 |
| I.PHOS | mmol/l | 0.2 | | 0.1 | 7.6 | | 2.9 |
| Mg2+ | mmol/l | 7.35 | | 6.90 | 4.00 | | 5.90 |
| Na/Crea | mM/mM | 7.60 | | 12.60 | 8.40 | | 18.10 |
| K/Crea | mM/mM | 15.80 | | 26.80 | 11.70 | | 8.60 |
| Cl/Crea | mM/mM | 6.20 | | 18.00 | 3.70 | | 16.50 |
| Ca/Crea | mM/mM | 0.60 | | 3.57 | 0.47 | | 1.53 |
| Pho/Crea | mM/mM | 0.01 | | 0.02 | 0.54 | | 0.36 |
| Mg/Crea | mM/mM | 0.40 | | 1.20 | 0.30 | | 0.70 |
| LDH/crea | IU/mM | 0.57 | | 1.78 | 1.28 | | 1.12 |
| NAG/crea | IU/mM | 0.03 | | 0.21 | 0.42 | | 0.63 |
| NTx/Crea | nME/mM | | 576 | 447 | | 634 | 705 |
| CTx/Crea | μg/μm. | | 1202 | 679 | | 1168 | 1394 |
| Pyr/Crea | nM/mM | | 163 | 241 | | 181 | 253 |

d −8, d −7 and d 13 indicate day −8, day −7 and day 13 relative to the starting day of dosing

The salmon calcitonin group presented with moderate decreases in serum somatomedin (S.MLD, see TABLES 9 and 10).

TABLE 9
Hormones - Males
Control

| Animal no. | | W62501 | | | W62502 | | |
|---|---|---|---|---|---|---|---|
| Test | Units | d −6 | d 7 | d 13 | d −6 | d 7 | d 13 |
| ACTH | pg/ml | 91 | 63 | 87 | 117 | 136 | 150 |
| CORTISOL | nmol/l | 2183 | 1415 | 1328 | 1378 | 1020 | 1348 |
| ALDOST | pg/ml | 316 | 433 | 484 | 501 | 644 | 622 |
| INSULIN | mU/l | 26.0 | 33.0 | 37.0 | 12.0 | 30.0 | 9.0 |
| GLUCAG | pg/ml | 791 | 486 | 704 | 577 | 353 | 585 |
| C-PEPTI | ng/ml | n/a | 5.20 | 5.50 | n/a | 3.60 | 1.60 |
| GASTRIN | pg/ml | n/a | 105 | 93 | n/a | 147 | 148 |
| T3 | nmol/l | 1.34 | 2.61 | 2.94 | 2.19 | 2.73 | 2.50 |
| T4 | nmol/l | 56 | 61 | 44 | 57 | 68 | 48 |
| TSH | mUI/l | 0.17 | 0.18 | 0.42 | 0.00 | 0.05 | 0.04 |
| IPH | pg/ml | 103 | 75 | 108 | 174 | 173 | 155 |
| CT | pg/ml | 5.9 | 4.6 | 4.8 | 16.4 | 15.0 | 13.1 |
| VD25-H | nmol/l | 49 | 47 | 54 | 76 | 71 | 58 |
| VD1-25dh | pmol/l | n/a | — | — | n/a | — | — |
| OSTEO | ng/ml | n/a | 26 | 34 | n/a | 41 | 40 |
| CTx | nmol/l | 10 | 15 | 20 | 17 | 19 | 20 |
| ICTP | ng/ml | 18 | 13 | 19 | 26 | 16 | 15 |
| PICP | ng/ml | n/a | 311 | 395 | n/a | 610 | 495 |
| G.H. | ng/ml | 13.8 | 7.0 | 16.2 | 15.2 | 3.6 | 17.2 |
| S.STA | pg/ml | n/a | — | — | n/a | — | — |
| S.MED | ng/ml | n/a | 888 | 1185 | n/a | 793 | 689 |
| PROLACT | ng/ml | 0.0 | 3.3 | 3.6 | 21.6 | 22.5 | 22.5 |
| TESTO | nmol/l | 10.5 | 8.4 | n.s. | 7.9 | 4.7 | n.s. |
| ESTR | nmol/l | n/a | n/a | n/a | n/a | n/a | n/a |
| PROG | pmol/l | n/a | n/a | n/a | n/a | n/a | n/a |

Salmon Calcitonin

| Animal no. | | W62503 | | | W62504 | | |
|---|---|---|---|---|---|---|---|
| Test | Units | d −6 | d 7 | d 13 | d −6 | d 7 | d 13 |
| ACTH | pg/ml | 98 | 87 | 87 | 115 | 78 | 73 |
| CORTISOL | nmol/l | 2316 | 979 | 1611 | 1578 | 1523 | 1709 |
| ALDOST | pg/ml | 983 | 1058 | 819 | 465 | 987 | 977 |
| INSULIN | mU/l | 13.0 | 14.0 | 17.0 | 4.0 | 10.0 | 22.0 |
| GLUCAG | pg/ml | 905 | 247 | 428 | 869 | 218 | 503 |
| C-PEPTI | ng/ml | n/a | 1.70 | 1.80 | n/a | 1.20 | 2.30 |
| GASTRIN | pg/ml | n/a | 83 | 88 | n/a | 128 | 136 |
| T3 | nmol/l | 1.06 | 2.35 | 2.51 | 1.48 | 1.65 | 1.90 |
| T4 | nmol/l | 53 | 64 | 47 | 62 | 79 | 65 |
| TSH | mUI/l | 0.99 | 1.12 | 1.03 | 0.14 | 0.41 | 0.40 |
| IPH | pg/ml | 213 | 75 | 78 | 99 | 62 | 71 |
| CT | pg/ml | 6.7 | 4.0 | 2.4 | 5.1 | 2.5 | 4.9 |
| VD25-H | nmol/l | 63 | 50 | 49 | 62 | 44 | 45 |
| VD1-25dh | pmol/l | n/a | — | — | n/a | — | — |
| OSTEO | ng/ml | n/a | 33 | 41 | n/a | 27 | 30 |
| CTx | nmol/l | 12 | 26 | 38 | 18 | 22 | 24 |
| ICTP | ng/ml | 21 | 15 | 15 | 22 | 21 | 20 |
| PICP | ng/ml | n/a | 284 | 363 | n/a | 361 | 439 |
| G.H. | ng/ml | 11.5 | 1.7 | 16.2 | 14.6 | 13.6 | 15.7 |
| S.STA | pg/ml | n/a | — | — | n/a | — | — |
| S.MED | ng/ml | n/a | 268 | 332 | n/a | 307 | 384 |
| PROLACT | ng/ml | 8.1 | 8.6 | 4.6 | 0.0 | 0.0 | 6.6 |
| TESTO | nmol/l | 8.5 | 3.6 | n.s. | 9.5 | 7.3 | n.s. |
| ESTR | nmol/l | n/a | n/a | n/a | n/a | n/a | n/a |
| PROG | pmol/l | n/a | n/a | n/a | n/a | n/a | n/a |

PTS893

| Animal no. | | W62505 | | | W62506 | | |
|---|---|---|---|---|---|---|---|
| Test | Units | d −6 | d 7 | d 13 | d −6 | d 7 | d 13 |
| ACTH | pg/ml | 96 | 101 | 83 | 115 | 88 | 91 |
| CORTISOL | nmol/l | 1662 | 1156 | 1299 | 1506 | 1432 | 1212 |
| ALDOST | pg/ml | 265 | 380 | 592 | 141 | 471 | 651 |
| INSULIN | mU/l | 16.0 | 22.0 | 14.0 | 12.0 | 38.0 | 10.0 |
| GLUCAG | pg/ml | 858 | 656 | 786 | 694 | 497 | 739 |
| C-PEPTI | ng/ml | n/a | 2.90 | 2.10 | n/a | 4.40 | 2.40 |
| GASTRIN | pg/ml | n/a | 84 | 78 | n/a | 98 | 94 |
| T3 | nmol/l | 2.48 | 3.47 | 3.55 | 1.38 | 2.76 | 2.43 |
| T4 | nmol/l | 84 | 90 | 68 | 59 | 80 | 56 |
| TSH | mUI/l | 0.22 | 0.40 | 0.15 | 0.00 | 0.07 | 0.03 |
| IPH | pg/ml | 123 | 96 | 78 | 71 | 62 | 55 |
| CT | pg/ml | 6.1 | 4.0 | 4.6 | 10.4 | 7.8 | 7.6 |
| VD25-H | nmol/l | 77 | 62 | 50 | 88 | 62 | 50 |
| VD1-25dh | pmol/l | n/a | — | — | n/a | — | — |
| OSTEO | ng/ml | n/a | 43 | 55 | n/a | 32 | 42 |
| CTx | nmol/l | 19 | 20 | 31 | 12 | 12 | 16 |
| ICTP | ng/ml | 28 | 23 | 22 | 18 | 16 | 18 |
| PICP | ng/ml | n/a | 420 | 500 | n/a | 774 | 706 |
| G.H. | ng/ml | 13.4 | 15.8 | 12.1 | 8.5 | 11.6 | 14.0 |
| S.STA | pg/ml | n/a | — | — | n/a | — | — |
| S.MED | ng/ml | n/a | 749 | 914 | n/a | 828 | 867 |
| PROLACT | ng/ml | 7.1 | 15.7 | 7.5 | 7.5 | 5.5 | 2.2 |
| TESTO | nmol/l | 11.8 | 10.5 | n.s. | 5.3 | 3.7 | n.s. |
| ESTR | nmol/l | n/a | n/a | n/a | n/a | n/a | n/a |
| PROG | pmol/l | n/a | n/a | n/a | n/a | n/a | n/a |

d −6, d 7 and d 13 indicate day −6, day 7 and day 13 relative to the starting day of dosing

TABLE 10
Hormones - Females
Control

| Animal no. | | W62551 | | | W62552 | | |
|---|---|---|---|---|---|---|---|
| Test | Units | d −8 | d 7 | d 13 | d −8 | d 7 | d 13 |
| ACTH | pg/ml | 146 | 276 | 121 | 58 | 60 | 101 |
| CORTISOL | nmol/l | 1983 | 1546 | 827 | 1894 | 837 | 818 |
| ALDOST | pg/ml | 244 | 953 | 312 | 149 | 90 | 199 |
| INSULIN | mU/l | 8.0 | 12.0 | 7.0 | 2.0 | 29.0 | 21.0 |
| GLUCAG | pg/ml | 729 | 779 | 583 | 818 | 507 | 514 |
| C-PEPTI | ng/ml | n/a | 2.40 | 1.40 | n/a | 3.30 | 2.30 |
| GASTRIN | pg/ml | n/a | 84 | 102 | n/a | 90 | 92 |
| T3 | nmol/l | 2.22 | 2.95 | 3.40 | 2.04 | 3.09 | 3.23 |
| T4 | nmol/l | 78 | 67 | 59 | 51 | 50 | 49 |
| TSH | mUI/l | 0.14 | 0.27 | 0.49 | 0.15 | 0.54 | 0.50 |
| IPH | pg/ml | 155 | 149 | 129 | 145 | 129 | 112 |
| CT | pg/ml | 4.70 | 3.90 | 4.10 | 11.50 | 11.60 | 11.20 |
| VD25-H | nmol/l | 64 | 59 | 51 | 80 | 78 | 70 |
| VD1-25dh | pmol/l | n/a | — | — | n/a | — | — |
| OSTEO | ng/ml | n/a | 37 | 39 | n/a | 34 | 39 |
| CTx | nmol/l | 11 | 26 | 28 | 12 | 16 | 20 |
| ICTP | ng/ml | 21 | 23 | 22 | 19 | 16 | 15 |
| PICP | ng/ml | n/a | 864 | 503 | n/a | 339 | 298 |
| G.H. | ng/ml | 8.5 | 13.4 | 1.7 | 7.0 | 12.0 | 4.5 |
| S.STA | pg/ml | n/a | — | — | n/a | — | — |
| S.MED | ng/ml | n/a | 696 | 839 | n/a | 1173 | 1527 |
| PROLACT | ng/ml | 4.30 | 8.30 | 5.90 | 2.90 | 0.00 | 0.00 |
| TESTO | nmol/l | | | | | | |
| ESTR | nmol/l | 58 | 64 | 61 | 48 | 45 | 60 |
| PROG | pmol/l | 3.40 | 3.50 | 1.70 | 2.70 | 1.10 | 1.40 |

Salmon Calcitonin

| Animal no. | | W62553 | | | W62554 | | |
|---|---|---|---|---|---|---|---|
| Test | Units | d −8 | d 7 | d 13 | d −8 | d 7 | d 13 |
| ACTH | pg/ml | 72 | 129 | 97 | 157 | 233 | 141 |
| CORTISOL | nmol/l | 1536 | 1220 | 1202 | 1222 | 1705 | 1128 |
| ALDOST | pg/ml | 185 | 948 | 523 | 155 | 1073 | 457 |
| INSULIN | mU/l | 12.0 | 8.0 | 9.0 | 20.0 | 18.0 | 24.0 |
| GLUCAG | pg/ml | 585 | 295 | 258 | 619 | 594 | 303 |
| C-PEPTI | ng/ml | n/a | 1.60 | 1.00 | n/a | 1.50 | 2.20 |
| GASTRIN | pg/ml | n/a | 83 | 84 | n/a | 91 | 84 |
| T3 | nmol/l | 1.17 | 1.68 | 1.51 | 1.43 | 1.51 | 2.00 |
| T4 | nmol/l | 58 | 76 | 60 | 61 | 87 | 60 |
| TSH | mUI/l | 0.81 | 1.31 | 1.16 | 0.08 | 0.34 | 0.41 |
| IPH | pg/ml | 59 | 47 | 58 | 145 | 82 | 53 |
| CT | pg/ml | 3.10 | 6.40 | 4.90 | 7.00 | 3.60 | 2.30 |
| VD25-H | nmol/l | 61 | 43 | 40 | 72 | 56 | 60 |
| VD1-25dh | pmol/l | n/a | — | — | n/a | — | — |
| OSTEO | ng/ml | n/a | 21 | 25 | n/a | 35 | 35 |
| CTx | nmol/l | 12 | 21 | 25 | 17 | 34 | 28 |
| ICTP | ng/ml | 28 | 28 | 24 | 29 | 30 | 24 |
| PICP | ng/ml | n/a | 115 | 142 | n/a | 240 | 287 |
| G.H. | ng/ml | 6.3 | 15.2 | 8.6 | 5.1 | 17.9 | 13.1 |
| S.STA | pg/ml | n/a | — | — | n/a | — | — |
| S.MED | ng/ml | n/a | 374 | 297 | n/a | 204 | 488 |
| PROLACT | ng/ml | 0.00 | 2.30 | 4.30 | 19.30 | 20.20 | 24.40 |
| TESTO | nmol/l | | | | | | |
| ESTR | nmol/l | 47 | 63 | 59 | 141 | 82 | 170 |
| PROG | pmol/l | 1.80 | 1.90 | 1.50 | 2.60 | 4.00 | 1.60 |

PTS893

| Animal no. | | W62555 | | | W62556 | | |
|---|---|---|---|---|---|---|---|
| Test | Units | d −8 | d 7 | d 13 | d −8 | d 7 | d 13 |
| ACTH | pg/ml | 109 | 104 | 110 | 95 | 132 | 126 |
| CORTISOL | nmol/l | 1482 | 1331 | 917 | 1532 | 1253 | 1375 |
| ALDOST | pg/ml | 314 | 217 | 330 | 210 | 228 | 226 |
| INSULIN | mU/l | 1.0 | 22.0 | 19.0 | 15.0 | 30.0 | 22.0 |
| GLUCAG | pg/ml | 711 | 591 | 657 | 696 | 437 | 380 |
| C-PEPTI | ng/ml | n/a | 3.00 | 2.40 | n/a | 3.80 | 3.50 |
| GASTRIN | pg/ml | n/a | 83 | 82 | n/a | 96 | 91 |
| T3 | nmol/l | 2.08 | 2.74 | 2.63 | 1.98 | 2.69 | 2.05 |
| T4 | nmol/l | 72 | 56 | 55 | 59 | 61 | 45 |
| TSH | mUI/l | 0.34 | 0.14 | 0.25 | 0.88 | 0.89 | 0.69 |
| IPH | pg/ml | 95 | 45 | 64 | 111 | 67 | 58 |
| CT | pg/ml | 2.50 | 1.90 | 2.70 | 1.80 | 2.90 | 2.80 |
| VD25-H | nmol/l | 72 | 53 | 47 | 55 | 44 | 43 |
| VD1-25dh | pmol/l | n/a | — | — | n/a | — | — |
| OSTEO | ng/ml | n/a | 38 | 43 | n/a | 32 | 36 |
| CTx | nmol/l | 13 | 11 | 15 | 17 | 14 | 14 |
| ICTP | ng/ml | 22 | 16 | 16 | 20 | 15 | 15 |
| PICP | ng/ml | n/a | 612 | 436 | n/a | 478 | 393 |
| G.H. | ng/ml | 3.5 | 1.5 | 0.0 | 1.1 | 8.2 | 11.8 |
| S.STA | pg/ml | n/a | — | — | n/a | — | — |
| S.MED | ng/ml | n/a | 533 | 502 | n/a | 432 | 589 |
| PROLACT | ng/ml | 0.00 | 0.20 | 3.20 | 9.90 | 5.70 | 3.60 |
| TESTO | nmol/l | | | | | | |
| ESTR | nmol/l | 67 | 68 | 60 | 59 | 66 | 57 |
| PROG | pmol/l | 2.80 | 1.70 | 1.50 | 2.40 | 2.20 | 2.40 |

d −8, d 7 and d 13 indicate day −8, day 7 and day 13 relative to the starting day of dosing

Tissue sampling. Animals were killed by deep anaesthesia induced by intravenous injection of Pentothal®, followed by exsanguinations. All relevant tissues were sampled for histopathology and gene expression profiling. The following tissue samples were processed for analysis: liver, kidney, pituitary, muscle, bone, duodenum, spleen and trachea. Samples for histopathology were fixed in phosphate-buffered 10% formalin. Bone demineralization was performed with 10% formic acid. Tissue samples were embedded in Paraplast® and sectioned at 4 microns, for staining with haematoxylin and eosin. Samples for gene expression profiling were quickly frozen in liquid nitrogen immediately after excision, stored on dry ice and subsequently in a deep-freezer at approximately −80° C. until further use. All selected tissues for gene expression profiling were examined histopathologically.

Histopathology. Histopathological examination of the tissues selected for gene profiling analysis exhibited a normal spectrum of incidental lesions which were in terms of severity and distribution of lesions not different to the controls in all groups of treatment.

A slightly higher incidence of inflammatory and regenerative changes in the kidneys of females administered salmon calcitonin was observed. These changes were not considered to be relevant, since no records of kidney toxicity exist after 40 years of calcitonin therapeutic use.

Bone sections were stained for osteonectin, osteopontin and osteocalcin and were evaluated histopathologically. Histomorphometry of the bone tissue was performed regarding parameters for bone resorption and synthesis (osteoid formation).

The osteonectin, osteopontin, and osteocalcin staining of the tibia showed no difference between the groups one (control) and two (salmon calcitonin). Osteonectin exhibited a major enlargement and deterioration of the epiphysial growth plate of animal no 2553 due to a severe non-treatment related pathological status (severe, subacute epiphysiolysis).

Histomorphometry of bone tissue was performed to determine parameters related to bone resorption and bone synthesis (osteoid formation).

The results (see, TABLES 11 and 12) showed that salmon calcitonin increased trabecular volume and thickness in about a 17% in tibia, but not in vertebra. PTS893 reduced the cortical thickness (18%) and increased the cortical porosity (54%) in tibia (T), but not in vertebra (V). In contrast, PTS893 induced an increase in osteoid volumne (37% T, 213% V) and surface (49% T, 37% V), as well as an increase in the osteoblast surface (40% T, 24% V), in both tibia and vertebra, respectively.

TABLE 11
Histomorphometry Tibia (Average Males and Females)

| | | Tb Th | Tb N | Tb Sp | | Ct Th | | | | |
|---|---|---|---|---|---|---|---|---|---|---|
| | BT/TV % | μm | mm⁻¹ | μm | Ct Por % | μm | OS/BS % | OV/BV % | ES/BS % | Obs/BS % |
| Control | 20.70 | 106.32 | 1.95 | 407.20 | 2.53 | 1583.13 | 40.00 | 8.76 | 5.73 | 17.53 |
| | 17.72 | 97.99 | 1.81 | 454.90 | 2.59 | 976.66 | 33.37 | 8.51 | 4.70 | 12.77 |
| | 28.74 | 109.18 | 2.63 | 270.69 | 1.21 | 1036.24 | 29.45 | 5.79 | 10.19 | 11.70 |
| | 20.15 | 103.59 | 1.94 | 410.62 | 1.19 | 1031.89 | 29.19 | 5.29 | 5.71 | 15.80 |
| mean | 21.83 | 104.27 | 2.08 | 385.85 | 1.88 | 1156.98 | 33.00 | 7.09 | 6.58 | 14.45 |
| SD | 4.79 | 4.77 | 0.37 | 79.79 | 0.78 | 285.39 | 5.04 | 1.80 | 2.45 | 2.69 |
| sCT | 32.28 | 140.64 | 2.30 | 295.01 | 2.10 | 895.98 | 42.71 | 11.72 | 5.02 | 18.32 |
| | 25.00 | 122.19 | 2.05 | 366.51 | 1.98 | 1022.55 | 31.58 | 5.86 | 2.31 | 6.37 |
| | 29.96 | 129.05 | 2.32 | 301.75 | 1.61 | 939.32 | 35.21 | 5.03 | 6.89 | 18.58 |
| | 16.08 | 115.65 | 1.39 | 603.45 | 2.40 | 1178.70 | 30.37 | 4.01 | 5.61 | 19.36 |
| mean | 25.83 | 126.88 | 2.01 | 391.68 | 2.02 | 1009.14 | 34.97 | 6.65 | 4.95 | 15.66 |
| SD | 7.17 | 10.68 | 0.43 | 144.81 | 0.33 | 124.65 | 5.56 | 3.46 | 1.93 | 6.21 |
| PTS893 | 19.69 | 129.22 | 1.52 | 526.99 | 2.76 | 1022.62 | 54.84 | 11.24 | 4.62 | 16.16 |
| | 16.65 | 93.20 | 1.79 | 466.69 | 2.94 | 893.43 | 43.57 | 9.61 | 4.76 | 21.25 |
| | 25.74 | 120.52 | 2.13 | 347.63 | 2.94 | 950.33 | 43.63 | 8.14 | 4.21 | 18.46 |
| | 24.78 | 126.07 | 1.97 | 382.61 | 2.95 | 939.53 | 54.97 | 9.95 | 2.85 | 25.25 |
| mean | 21.72 | 117.25 | 1.85 | 430.98 | 2.90 | 951.48 | 49.25 | 9.74 | 4.11 | 20.28 |
| SD | 4.30 | 16.43 | 0.26 | 81.20 | 0.09 | 53.46 | 6.53 | 1.28 | 0.87 | 3.91 |

sCT: salmon Calcitonin;
SD: Standard deviation
BV/TV trabecular bone volume;
Tb. Th. Trabecular thickness;
Tb. N. Trabecular number;
Tb. Sp. Trabecular Separation;
Ct. Por. Cortical porosity;
Ct, Th. Cortical thickness;
OS/BS osteoid surface;
OV/BV osteoid volume;
ES/BS eroded surface;
Obs/BS osteoblast surface.

TABLE 12
Histomorphometry Vertebra (Average Males and Females)

| | | Tb Th | Tb N | Tb Sp | | Ct Th | | | | |
|---|---|---|---|---|---|---|---|---|---|---|
| | BT/TV % | μm | mm⁻¹ | μm | Ct Por % | μm | OS/BS % | OV/BV % | ES/BS % | Obs/BS % |
| Control | 21.67 | 179.80 | 1.21 | 649.79 | 0.88 | 887.91 | 23.61 | 1.22 | 8.94 | 16.14 |
| | 15.85 | 144.89 | 1.09 | 769.35 | 0.26 | 639.93 | 20.77 | 2.02 | 8.81 | 5.96 |
| | 19.54 | 122.91 | 1.59 | 506.23 | 0.87 | 416.48 | 17.91 | 1.58 | 5.85 | 4.07 |
| | 21.95 | 131.30 | 1.67 | 466.91 | 0.85 | 604.45 | 11.58 | 0.97 | 1.82 | 4.79 |
| mean | 19.75 | 144.72 | 1.39 | 598.07 | 0.71 | 637.20 | 18.47 | 1.45 | 6.36 | 7.74 |
| SD | 2.82 | 25.07 | 0.28 | 138.62 | 0.30 | 193.78 | 5.15 | 0.45 | 3.34 | 5.65 |
| sCT | 17.32 | 113.29 | 1.53 | 540.84 | 1.70 | 705.10 | 3.95 | 0.46 | 11.60 | 3.21 |
| | 19.33 | 144.31 | 1.34 | 602.15 | 1.18 | 810.09 | 5.82 | 0.86 | 2.55 | 3.97 |
| | 20.11 | 118.49 | 1.70 | 470.71 | 1.18 | 576.42 | 11.48 | 1.43 | 4.93 | 6.81 |
| | 19.46 | 123.71 | 1.57 | 511.96 | 0.12 | 907.16 | 4.91 | 0.32 | 3.47 | 1.23 |
| mean | 19.06 | 124.95 | 1.53 | 531.42 | 1.05 | 749.69 | 6.54 | 0.77 | 5.64 | 3.80 |
| SD | 1.21 | 13.59 | 0.15 | 55.24 | 0.66 | 141.96 | 3.38 | 0.50 | 4.09 | 2.31 |
| PTS893 | 15.15 | 105.46 | 1.44 | 590.67 | 1.49 | 707.43 | 18.84 | 3.24 | 9.31 | 10.36 |
| | 20.23 | 118.79 | 1.70 | 468.39 | 1.45 | 629.35 | 41.28 | 8.42 | 2.30 | 9.07 |
| | 23.56 | 134.66 | 1.75 | 436.79 | 0.41 | 740.87 | 23.65 | 3.49 | 2.55 | 10.47 |
| | 24.86 | 134.82 | 1.84 | 407.56 | 0.92 | 624.35 | 17.66 | 2.66 | 3.96 | 8.33 |
| mean | 20.95 | 123.43 | 1.68 | 475.85 | 1.07 | 675.50 | 25.36 | 4.45 | 4.53 | 9.56 |
| SD | 4.33 | 14.15 | 0.17 | 80.47 | 0.51 | 57.85 | 10.93 | 2.67 | 3.27 | 1.04 |

sCT: salmon Calcitonin;
SD: Standard deviation
BV/TV trabecular bone volume;
Tb. Th. Trabecular thickness;
Tb. N. Trabecular number;
Tb. Sp. Trabecular Separation;
Ct. Por. Cortical porosity;
Ct, Th. Cortical thickness;
OS/BS osteoid surface;
OV/BV osteoid volume;
ES/BS eroded surface;
Obs/BS osteoblast surface.

Histomorphometry showed inconsistent results between tibial and vertebral bone, except for an increase in osteoid synthesis induced by PTS893. This effect is well documented for parathyroid hormone, when administered in a discontinuous way.

RNA extraction andpurificationz. A set of tissues was selected for gene expression profiling. These set included samples from kidney, bone, muscle, duodenum, pituitary and liver. In particular, diaphyseal bone from femur and tibia were processed for gene expression profiling. Briefly, total RNA was obtained by acid guanidinium thiocyanate-phenol-chloroform extraction (Trizol®, Invitrogen Life Technologies, Carlsbad, Calif. USA) from each frozen tissue section and the total RNA was then purified on an affinity resin (RNeasy®, Qiagen) according to the manufacturer's instructions. Total RNA was quantified by the absorbance at λ=260 nm (A260 nm), and the purity was estimated by the ratio A260 nm/A280 nm. Integrity of the RNA molecules was confirmed by non-denaturing agarose gel electrophoresis. RNA was stored at approximately −80° C. until analysis. One part of each individual RNA sample was kept for the analysis of critical genes by means of Real-time PCR.

Hybridization assay. Transcript profiling by means of GeneChip® expression probe arrays was done in the laboratories of the Genomics Factory EU, as recommended by the manufacturer of the GeneChip® system (GeneChip Expression Analysis Technical Manual, Affymetrix Inc., Santa Clara, Calif. USA). HG-U95Av2 GeneChip® expression probe arrays (Affymetrix, Santa Clara Calif. USA) were used. Double stranded cDNA was synthesized with a starting amount of approximately 5 μg full-length total RNA using the Superscript Choice System (Invitrogen Life Technologies) in the presence of a T7-(dT) 24 DNA oligonucleotide primer. Following synthesis, the cDNA was purified by phenol/chloroform/isoamylalcohol extraction and ethanol precipitation. The purified cDNA was then transcribed in vitro using the BioArray® High Yield RNA Transcript Labelling Kit (ENZO) in the presence of biotinylated ribonucleotides form biotin labelled cRNA. The labelled cRNA was then purified on an affinity resin (Rneasy®, Qiagen), quantified and fragmented. An amount of approximately 10 μg labelled cRNA was hybridized for approximately 16 hours at 45° C. to an expression probe array. The array was then washed and stained twice with streptavidin-phycoerythrin (Molecular Probes) using the GeneChip Fluidics Workstation 400 (Affymetrix). The array was then scanned twice using a confocal laser scanner (GeneArray® Scanner, Agilent) resulting in one scanned image. This resulting “.data-file” was processed using the Micro Array Analysis Suite version 4 (MAS4) program (Affymetrix) into a “.cel-file”. The “.cel file” was captured and loaded into the Affymetrix GeneChip Laboratory Information Management System (LIMS). The LIMS database is connected to a UNIX Sun Solaris server through a network filing system that allows for the average intensities for all probes cells (CEL file) to be downloaded into an Oracle database. Raw data was converted to expression levels using a “target intensity” of 150. The numerical values displayed are weighted averages of the signal intensities of the probe-pairs comprised in a probe-set for a given transcript sequence (AvgDiff value). The data were checked for quality and loaded into the GeneSpring® software versions 4.2.4 and 5 (Silicon Genetics, Calif. USA) for analysis.

Data analysis. Data analysis was performed with the Silicon Genetics software package GeneSpring version 4.2.1 and 5. Average difference values below 20 were set to 20. Various filtering and clustering tools in these programs were used to explore the data sets and identify transcript level changes that inform on altered cellular and tissue functions and that can be used to establish working hypotheses on the modes of action of the compound.

The threshold range for considering as up or down regulation was determined within the context of the biological interpretation of the EXAMPLE.

The information content of these data sets is a conjunction of numerical changes and biological information. The decision to consider a specific gene relevant was based on a conjunction of numerical changes identified by comparative and statistical algorithms and the relationship to other modulated genes that point to a common biological theme. The weight of that relationship was assessed by the analyst through a review of the relevant scientific literature.

Increase and decrease reported here refer to transcript abundance, unless specifically stated.

Gene expression profiling. Multi-organ comparative gene profiling analysis was performed in the group administered salmon calcitonin at 50 μg/animnal/day. The organs chosen for analysis were liver, kidney, pituitary, skeletal muscle, bone, duodenum, spleen and trachea.

TABLE 13
Multi-Organ Gene Expression Profiling of Salmon Calcitonin

| GeneChip ® | | | | | | | |
|---|---|---|---|---|---|---|---|
| expression probe | | | | | | | |
| set identifier | Coding Gene | bone | kidney | liver | muscle | pituitary | trachea |
| 36611_at | acid phosphatase 1 isoform a | | | −1.33 | | −1.33 | |
| 32714_s_at | activin A receptor type II-like 1 | −1.62 | | | −1.83 | | |
| 39314_at | activin A type IIB receptor precursor | | | −1.12 | 1.41 | −4.15 | |
| 35915_at | activin beta-C chain. | −1.21 | | | −2.41 | −1.67 | |
| 36621_at | alpha-2-HS-glycoprotein | 1.33 | 1.53 | | | | 1.12 |
| 34588_i_at | amelogenin | | | −1.61 | | | |
| 37747_at | annexin V | | −1.30 | 1.87 | | −2.58 | |
| 40376_at | arylsulfatase E precursor | | −1.59 | | | | |
| 39326_at | ATPase H(+)-vacuolar | −1.57 | −2.80 | | | −1.62 | |
| 38814_at | ATFase H(+)-vacuolar subunit | 1.22 | | | | | |
| 33741_at | ATPase, H+ transport, lysosomal | 1.23 | | | | −1.50 | |
| 33033_at | ATPase, H+ transporting, lysosomal | −1.29 | | −3.19 | | −1.43 | 1.23 |
| 38814_at | ATPase, H+ transporting, lysosomal | | | | 1.30 | −1.28 | 1.14 |
| 38126_at | biglycan | | | | | 1.75 | −1.61 |
| 39407_at | bone morphogenetic protein 1 | | | −1.20 | | −1.55 | |
| 31399_at | bone morphogenetic protein 10 | 1.44 | 1.45 | −1.31 | −1.77 | | |
| 1113_at | bone morphogenetic protein 2A | −1.12 | 2.63 | | | | 1.29 |
| 1831_at | bone morphogenetic protein 5 | −1.43 | 1.39 | 1.40 | | | |
| 1733_at | bone morphogenetic protein 6 | | −1.37 | −1.17 | −1.64 | −1.27 | −1.1 |
| | precursor | | | | | | |
| 34500_at | calcium binding protein 1 (calbrain) | | 2.31 | | | 1.21 | |
| 31670_s_at | calcium/calmodulin-dependent protein | | 1.17 | | 1.57 | −1.28 | 1.60 |
| | kinase (CaM kinase) II gamma | | | | | | |
| 1751_g_at | calreticulin | −4.03 | −1.60 | | 1.67 | | |
| 32067_at | cAMP responsive element modulator | 1.39 | | −1.24 | | −1.50 | |
| | (CREM) | | | | | | |
| 39241_at | carbonic anhydrase I | −2.68 | | 1.18 | | −1.69 | |
| 40095_at | carbonic anhydrase II | −1.69 | | | | | |
| 40163_r_at | cartilage oligomeric matrix protein | 2.36 | 5.61 | | | | |
| | precursor | | | | | | |
| 128_at | cathepsin k | 1.18 | | | 1.35 | −2.33 | |
| 129_g_at | cathepsin k | 1.20 | −1.54 | | 1.17 | −1.28 | |
| 38466_at | cathepsin k | 1.27 | | | 1.40 | | −1.19 |
| 40718_at | cathepsin w | −1.31 | | −1.54 | | 2.05 | |
| 32833_at | CDC-like kinase 1 | 1.63 | | | | | |
| 646_s_at | CDC-like kinase 2 isoform hclk2/139 | 1.19 | | | 1.86 | | |
| 38112_g_at | chondroitin sulphate proteoglycan 2 | | −2.16 | | 1.51 | | −1.68 |
| | (versican) | | | | | | |
| 32642_at | chondroitin sulphate proteoglycan 3 | | | | | −1.49 | |
| | (neurocan) | | | | | | |
| 31493_s_at | chorionic somatomammotropin | | | | | −1.59 | |
| | hormone 1 | | | | | | |
| 40714_at | chymotrypsin C (caldecrin) | | | | | 1.39 | 3.22 |
| 35474_s_at | Collagen type 1 and PDGFB fusion | | | | −7.30 | | −3.35 |
| | transcript | | | | | | |
| 598_at | collagen type II alpha-1 | −1.38 | | 1.69 | −1.27 | 2.77 | −3.02 |
| 32488_at | collagen type III alpha 1 | −1.41 | −1.59 | −1.53 | −3.20 | −1.89 | −1.35 |
| 38952_s_at | collagen type IV alpha-2 | 1.23 | | | | −1.73 | |
| 35379_at | collagen type IX alpha1 | −2.22 | −3.28 | | | | |
| 38722_at | collagen type VI alpha-1 | | −3.38 | −1.13 | −1.42 | | |
| 34802_at | collagen type VI alpha-2 (AA 570-998) | −1.37 | | −1.10 | −1.39 | | −1.28 |
| 37892_at | collagen type XI alpha-1 | 1.24 | | | | −2.46 | −1.51 |
| 1026_s_at | collagen type XI alpha2 | −1.20 | −1.32 | | | 1.15 | −2.20 |
| 1027_at | collagen type XI alpha2 | 1.11 | | −1.25 | | | 1.37 |
| 32305_at | collagen, type I, alpha 2 | | −1.45 | | | | −1.54 |
| 39333_at | collagen, type IV, alpha 1 | | | | | | −1.49 |
| 39925_at | collagen, type IX, alpha 2 | | | | | −2.38 | −1.36 |
| 38420_at | collagen, type V, alpha 2 | | −1.29 | −1.18 | −1.11 | | −1.10 |
| 41351_at | collagen, type VI, alpha 1 | | −2.29 | | −1.27 | −1.50 | |
| 41350_at | collagen, type VI, alpha 1 precursor | | | | | | −3.55 |
| 35168_f_at | collagen, type XVI, alpha 1 | | | | | | −1.59 |
| 35169_at | collagen, type XVI, alpha 1 | | | | | | −1.18 |
| 39632_at | collagenase 3 (matrix metalloproteinase | 1.20 | | | | | |
| | 13) | | | | | | |
| 36638_at | connective tissue growth factor | | | | | −2.11 | |
| 40697_at | cyclin A2 | −1.60 | | | | | |
| 34736_at | cyclin B1 | −2.83 | | | | | |
| 36650_at | cyclin D2 | 1.21 | | | | | |
| 35249_at | cyclin E2 | −2.95 | | | | | |
| 1206_at | cyclin-dependent kinase 5 | 1.56 | | | | | −1.54 |
| 799_at | cyclin-dependent kinase 5, regulatory | 1.32 | | | | | |
| | subunit 1 (p35) | | | | | | |
| 41546_at | cyclin-dependent kinase 6 | 1.15 | 1.52 | | | 1.34 | |
| 2031_s_at | cyclin-dependent kinase inhibitor 1A | 1.95 | | | | | |
| | (p21, Cip1) | | | | | | |
| 35816_at | cystatin B (stefin B) | 1.57 | | | | | |
| 806_at | cytokine-inducible kinase | 1.20 | | | | 1.35 | |
| 40049_at | death-associated protein kinase 1 | | | | | −1.47 | −1.29 |
| 33903_at | death-associated protein kinase 3 | −1.22 | | | | | |
| 34029_at | dentin matrix acidic phosphoprotein 1 | 1.65 | | | | | |
| | (DMP1) | | | | | | |
| 40186_at | dual specificity phosphatase 9 | | | | | | 1.59 |
| 37996_s_at | dystrophia myotonica-protein kinase | | 1.25 | | | | −1.50 |
| 342_at | ectonucleotide Pyrophosphatase/ | 1.45 | | | | | |
| | Phosphodiesterase 1; | | | | | | |
| 343_s_at | ectonucleotide pyrophosphatase/ | 1.11 | | | −1.42 | | |
| | phosphodiesterase 1; | | | | | | |
| 33602_at | endothelial differentiation, G protein | | 1.15 | | | 2.24 | −1.66 |
| | coupled receptor 6 precursor | | | | | | |
| 1442_at | oestrogen receptor | 1.47 | 1.23 | | | 1.60 | |
| 33670_at | oestrogen receptor | 1.30 | | | | | |
| 1487_at | oestrogen receptor-related protein | 1.11 | | | −1.52 | | 1.24 |
| 38882_r_at | oestrogen-responsive B box protein | 1.22 | | −1.51 | | | |
| | (EBBP) | | | | | | |
| 39945_at | fibroblast activation protein | −1.27 | | | | −1.48 | −1.32 |
| 996_at | fibroblast growth factor 1 (acidic) | | 1.17 | | −1.41 | | |
| 41586_at | fibroblast growth factor 18 | | 2.06 | | | | |
| 1730_s_at | fibroblast growth factor 4 | | 1.55 | | | | 1.46 |
| 424_s_at | fibroblast growth factor receptor. | −1.17 | −1.59 | | | | |
| 40131_at | follistatin-like 1 | −1.31 | | | | | |
| 40132_g_at | follistatin-like 1 | | | | | −1.22 | 1.15 |
| 33510_s_at | glutamate receptor, metabotropic 1 | 1.26 | | | −1.31 | | |
| 33269_at | GPI1 N-acetylglucosaminyl transferase | 1.24 | | | | | |
| | component Gpi1 | | | | | | |
| 1401_g_at | granulocyte-macrophage | −3.07 | 2.24 | | | | |
| | colony-stimulating factor (CSF1) | | | | | | |
| 1911_s_at | growth arrest and | | 1.84 | | −3.84 | | 1.24 |
| | DNA-damage-inducible, alpha | | | | | | |
| 37615_at | growth factor receptor-bound protein | 1.21 | | | | −1.61 | |
| | 10 | | | | | | |
| 32845_at | heparan sulphate proteoglycan 2 | | | 1.27 | | | −1.11 |
| | (perlecan) | | | | | | |
| 32778_at | inositol 1,4,5-triphosphate receptor, | | 1.75 | | | −2.57 | 1.20 |
| | type 1 | | | | | | |
| 32779_s_at | inositol 1,4,5-triphosphate receptor, | | | 1.21 | 2.02 | | |
| | type 1 | | | | | | |
| 756_at | inositol 1,4,5-triphosphate receptor, | | | | | | 1.24 |
| | type 2 | | | | | | |
| 34209_at | inositol 1,4,5-trisphosphate 3-kinase | | 2.29 | | 1.42 | −1.36 | 1.75 |
| | isoenzyme | | | | | | |
| 33506_at | inositol polyphosphate 4-phosphatase | 1.12 | 1.66 | 2.09 | | 1.27 | |
| | type I-beta | | | | | | |
| 172_at | inositol polyphosphate-5-phosphatase, | | | −1.22 | −1.15 | | |
| 32697_at | inositol(myo)-1(or | −1.36 | −2.70 | | | 1.61 | |
| | 4)-monophosphatase 1 | | | | | | |
| 36496_at | inositol(myo)-1(or | | | | | | 1.13 |
| | 4)-monophosphatase 2 | | | | | | |
| 2079_s_at | insulin-like growth factor (IGF-II) | | | | −1.32 | 1.15 | −1.31 |
| 36782_s_at | insulin-like growth factor 2 | | | | | | −1.69 |
| | (somatomedin A) | | | | | | |
| 1232_s_at | insulin-like growth factor binding | −1.31 | | | −1.53 | | |
| | protein | | | | | | |
| 40422_at | insulin-like growth factor binding | | | | | −2.97 | −1.16 |
| | protein 2 | | | | | | |
| 1586_at | insulin-like growth factor binding | 1.45 | | −1.16 | 1.70 | | |
| | protein 3 | | | | | | |
| 37319_at | insulin-like growth factor binding | 2.17 | | | 1.58 | | −1.52 |
| | protein 3 | | | | | | |
| 41420_at | insulin-like growth factor binding | | 1.15 | | −2.66 | | |
| | protein 5 | | | | | | |
| 1741_s_at | insulin-like growth factor binding | −2.49 | | | | −2.17 | −1.22 |
| | protein-2 | | | | | | |
| 1464_at | insulin-like growth factor II precursor | 1.18 | 1.10 | | | −1.26 | |
| 1591_s_at | insulin-like growth factor II precursor | 1.41 | | | | | −2.80 |
| 33082_at | integrin alpha 10 subunit | 1.33 | | | −2.32 | −1.18 | |
| 1100_at | interleukin-1 receptor-associated kinase | | | | 1.39 | −1.48 | |
| 2005_s_at | Janus kinase 3 | | | | −1.51 | | 1.57 |
| 40060_r_at | LIM protein (similar to rat protein | 1.44 | | | | −1.68 | −1.31 |
| | kinase C-binding enigma) | | | | | | |
| 36811_at | lysyl oxidase-like protein | −1.44 | 1.14 | | 1.30 | −1.19 | |
| 1433_g_at | MAD, mothers against decapentaplegic | 1.14 | −1.13 | | −1.61 | −1.65 | −1.69 |
| | homolog 3 | | | | | | |
| 34655_at | MAGUKs (membrane-associated | 1.23 | | | | | |
| | guanylate kinase homologues | | | | | | |
| 35652_g_at | MAP kinase kinase kinase (MTK1) | 1.14 | | | | | |
| 33246_at | MAPK13: mitogen-activated protein | | | −1.24 | −1.13 | −1.91 | 1.65 |
| | kinase 13 | | | | | | |
| 41280_r_at | MAPK8IP1: mitogen-activated protein | −1.31 | 1.92 | | | 1.58 | |
| | kinase 8 interacting protein 1 | | | | | | |
| 2004_at | MEK kinase | | | | 1.13 | −1.62 | 1.16 |
| 1509_at | metalloproteinase | −1.42 | | −1.11 | | −1.23 | −1.18 |
| 976_s_at | mitogen-activated protein kinase 1 | −1.61 | | | | | |
| 34006_s_at | mitogen-activated protein kinase 8 | 1.32 | | | | | |
| 1844_s_at | mitogen-activated protein kinase kinase 1 | | | | | −1.60 | 1.15 |
| 35694_at | mitogen-activated protein kinase kinase | | | | 1.26 | | |
| | kinase kinase 4 | | | | | | |
| 1469_at | mitogen-activated protein | | 1.13 | −1.30 | | | 1.16 |
| | kinase-activated protein kinase 2 | | | | | | |
| 1637_at | mitogen-activated protein | | | | 1.11 | | 1.34 |
| | kinase-activated protein kinase 3 | | | | | | |
| 37565_at | MMD: monocyte to macrophage | 1.28 | −2.48 | | | | −1.28 |
| | differentiation-associated | | | | | | |
| 38307_at | neurochondrin, | | | 2.80 | | −1.39 | |
| 39144_at | nuclear factor of activated T-cells, | | 2.72 | | 1.42 | | −1.70 |
| | cytoplasmic, calcineurin-dependent 1 | | | | | | |
| 41202_s_at | OS-4 protein (OS-4) | 1.24 | | | | −1.72 | |
| 1451_s_at | OSF-2os osteoblast specific factor-2 | −1.65 | | | | −2.06 | 1.56 |
| | (periostin) | | | | | | |
| 467_at | osteoclast stimulating factor (OSF) | −1.23 | −1.50 | −1.58 | | −4.12 | |
| 33814_at | PAK4 | 1.16 | | | −1.33 | 1.11 | |
| 38757_at | PDGF associated protein. | −1.89 | | | | −1.15 | 1.20 |
| 146_at | phosphatidylinositol 4-kinase, catalytic, | | | | 1.19 | | 1.23 |
| | beta polypeptide | | | | | | |
| 34496_at | phosphatidylinositol glycan, class L | | 2.34 | 1.34 | 1.51 | | |
| 34169_s_at | phosphatidylinositol polyphosphate | | | | | −1.33 | 1.49 |
| | 5-phosphotase, isoform b | | | | | | |
| 37412_at | phosphatidylinositol-4-phosphate | −1.87 | | −1.31 | | | |
| | 5-kinase isoform C (−1) | | | | | | |
| 37253_at | phosphatidylinositol-4-phosphate | | 1.17 | | −1.13 | | 1.11 |
| | 5-kinase, type I, beta | | | | | | |
| 35741_at | phosphatidylinositol-4-phosphate | | | | −1.18 | | −1.18 |
| | 5-kinase, type II, beta | | | | | | |
| 751_at | phosphatidylinositol-glycan-class C | 1.14 | 1.19 | | | −1.22 | |
| | (PIG-C) | | | | | | |
| 666_at | phosphodiesterase 4A, cAMP-specific | 1.33 | | −1.32 | | −1.18 | |
| 38526_at | phosphodiesterase 4D, cAMP-specific | 1.30 | 1.15 | | | | 3.51 |
| | (dunce (Drosophila)-homolog | | | | | | |
| | phosphodiesterase E3) | | | | | | |
| 38921_at | phosphodiesterase IB, | 1.52 | | | | 1.42 | 1.12 |
| | calmodulin-dependent | | | | | | |
| 31699_at | phosphoinositide-3-kinase | 1.56 | | −1.56 | | | |
| 36287_at | phosphoinositide-3-kinase, catalytic, | 1.31 | | | | | |
| | gamma polypeptide | | | | | | |
| 35665_at | phosphoinositide-3-kinase, class 3 | | | | | −1.11 | 1.21 |
| 364_s_at | phospholipase C b3 | 1.22 | | | | | |
| 901_g_at | phospholipase C, beta 4 | −1.20 | | 1.41 | | −1.55 | |
| 1293_s_at | phospholipase D | −1.26 | | | | | |
| 38023_at | phosphotidylinositol transfer protein | | 2.25 | | 1.33 | 1.55 | 1.71 |
| 38269_at | PKD2 Protein kinase D2 | | | 1.34 | | | |
| 32306_g_at | preprocollagen type I alpha-2 | 1.19 | | −1.38 | | −1.75 | −1.31 |
| 35473_at | preprocollagen type I alpha1. | −2.72 | −1.37 | | −3.94 | | −2.70 |
| 32307_s_at | procollagen | 1.13 | | −1.26 | −2.44 | −1.56 | −1.82 |
| 37605_at | procollagen alpha 1 type II | | | | −1.84 | | −1.61 |
| 36184_at | procollagen-lysine 5-dioxygenase | | 2.52 | | −2.15 | | −1.30 |
| 37037_at | procollagen-proline, 2-oxoglutarate | | | 1.87 | 1.46 | −1.67 | 1.29 |
| | 4-dioxygenase (proline 4-hydroxylase), | | | | | | |
| | alpha polypeptide I | | | | | | |
| 37633_s_at | progestagen-associated endometrial | | 2.00 | | | | |
| | protein (placental protein 14, | | | | | | |
| | pregnancy-associated endometrial | | | | | | |
| | alpha-2-globulin, alpha uterine protein) | | | | | | |
| 36109_at | prolidase (imidodipeptidase) PEPD: | −2.55 | | | | −2.05 | |
| 1884_s_at | proliferating cell nuclear antigen | −1.85 | | | | | |
| 36666_at | prolyl 4-hydroxylase beta | 1.95 | | | 1.37 | 2.08 | |
| 718_at | protease, serine, 11 (IGF binding) | −1.30 | | | | −1.81 | −1.30 |
| 719_g_at | protease, serine, 11 (IGF binding) | −1.43 | | | | −1.97 | −1.27 |
| 385_at | proteasome (prosome, macropain) | 1.36 | | −1.29 | | | |
| | subunit, beta type, 10 | | | | | | |
| 37431_at | protein inhibitor of activated STAT X | | | | | −1.23 | 1.28 |
| 39183_at | protein kinase 1 PCTAIRE | −1.17 | | | | | |
| 39711_at | protein kinase C substrate 80K-H | | | | | | 1.31 |
| 1437_at | protein kinase C, alpha | | | −2.06 | | | 1.82 |
| 36359_at | protein kinase, cAMP-dependent, | 1.39 | | 1.14 | −1.49 | 1.30 | 1.13 |
| | catalytic, gamma | | | | | | |
| 1091_at | protein kinase, cAMP-dependent, | 1.65 | −1.80 | | 2.06 | | |
| | regulatory, type I, beta | | | | | | |
| 116_at | protein kinase, cAMP-dependent, | 1.28 | | | −1.18 | | |
| | regulatory, type II, alpha | | | | | | |
| 33633_at | purinergic receptor P2Y, G-protein | 1.90 | −1.82 | | | | |
| | coupled, 11 | | | | | | |
| 32737_at | RAC2 Ras-related C3 botulinum toxin | 1.16 | | | | | 1.22 |
| | substrate 2 (rho family, small GTP | | | | | | |
| | binding protein Rac2) | | | | | | |
| 1007_s_at | receptor tyrosine kinase DDR | 1.21 | | | | | |
| 1048_at | retinoid X receptor-gamma | 1.47 | | | | 1.47 | |
| 41404_at | ribosomal protein S6 kinase | −1.67 | −1.40 | | −1.83 | | −1.40 |
| 865_at | ribosomal protein S6 kinase, 90 kD, | | −1.42 | | | | 1.27 |
| | polypeptide 3 | | | | | | |
| 32290_at | SCAMP1: secretory carrier membrane | 2.50 | | | | −1.27 | −1.39 |
| | protein 1 (vesicular transport) | | | | | | |
| 34342_s_at | secreted phosphoprotein 1 | | 1.15 | | | | −3.01 |
| | (osteopontin, bone sialoprotein I, early | | | | | | |
| | T-lymphocyte activation 1) | | | | | | |
| 39166_s_at | serine (or cysteine) proteinase | | −2.82 | 1.56 | 2.04 | | −1.29 |
| | inhibitor, clade H (heat shock protein | | | | | | |
| | 47), member 2 | | | | | | |
| 36217_at | serine/threonine kinase 38 | 1.54 | | | | | −1.59 |
| 1223_at | serine/threonine protein kinase | | | 2.42 | | | |
| 32447_at | SF-1; Steroidogenic factor-1 | 8.76 | | 1.59 | | 1.27 | −2.01 |
| 33338_at | signal transducer and activator of | | | −1.14 | 1.15 | −2.11 | −1.93 |
| | transcription 1 | | | | | | |
| 1244_at | signal transducer and activator of | | | | | | 1.57 |
| | transcription 2, 113 kD | | | | | | |
| 40458_at | signal transducer and activator of | | | | | 1.14 | 1.39 |
| | transcription 5A | | | | | | |
| 506_s_at | signal transducer and activator of | | | | 1.32 | 2.60 | |
| | transcription 5A | | | | | | |
| 41222_at | signal transducer and activator of | 1.44 | | | 1.14 | −1.46 | |
| | transcription 6 (STAT6) | | | | | | |
| 1950_s_at | Smad 3 | | | | −2.44 | | −1.16 |
| 38889_at | Smad anchor for receptor activation, | | 1.28 | | | −1.14 | −1.51 |
| | isoform 1 | | | | | | |
| 1013_at | Smad5 | | | | | −2.62 | 1.22 |
| 1955_s_at | SMAD6 (inhibits BMP/Smad1 | 1.19 | | | | −1.37 | |
| | (MADH1) | | | | | | |
| 37718_at | SNF-1 related kinase | 1.49 | | −1.13 | 1.18 | | |
| 35883_at | Spi-B transcription factor (SPI1/PU.1 | | 3.76 | | | −2.96 | 1.15 |
| | related) | | | | | | |
| 472_at | Stat5b (stat5b) | −1.42 | −1.28 | | | −1.83 | −2.50 |
| 38669_at | Ste20-related serine/threonine kinase | 1.24 | | | | −1.78 | |
| 38374_at | TEIG; TGFB inducible early growth | 1.18 | | | | −1.79 | |
| | response | | | | | | |
| 224_at | TGFB inducible early growth response; | 1.26 | | | | −2.69 | |
| | TIEG | | | | | | |
| 36940_at | TGFB1-induced anti-apoptotic factor 1 | 1.22 | | | 1.28 | −1.38 | |
| 32217_at | TGF-beta induced apoptosis protein 12 | 1.40 | 1.55 | | 1.12 | | |
| 41445_at | TGF-beta precursor | 1.14 | 1.11 | | | | |
| 1890_at | TGF-beta superfamily protein | 1.74 | 1.85 | 1.12 | | 1.38 | |
| 40631_at | Tob | −1.14 | | | 1.28 | −2.09 | |
| 32219_at | tousled-like kinase 1 | | −1.16 | | | | |
| 1897_at | transforming growth factor, beta | | 1.18 | | | | 1.12 |
| | receptor III (betaglycan, 300 kD) | | | | | | |
| 1735_g_at | transforming growth factor-beta 3 | | −1.15 | | −4.45 | −1.39 | −2.23 |
| 1767_s_at | transforming growth factor-beta 3 | −1.71 | 1.41 | | −1.71 | | |
| | (TGF-beta 3) | | | | | | |
| 40581_at | TRIO: triple functional domain | 1.65 | 1.62 | | 1.34 | | −1.42 |
| | (PTPRF interacting) | | | | | | |
| 32272_at | tubulin alpha | −1.20 | | | 1.18 | | |
| 330_s_at | tubulin alpha 1 | −1.80 | | 1.23 | −1.20 | −1.19 | |
| 40567_at | tubulin alpha 3 | −1.39 | | −1.18 | | | −1.10 |
| 685_f_at | tubulin alpha isotype H2-alpha | −4.36 | | 1.32 | | 2.13 | |
| 151_s_at | tubulin beta | −1.40 | −1.14 | 1.16 | 1.22 | 1.16 | |
| 33678_i_at | tubulin beta 2 | −1.15 | | | 1.75 | | |
| 33679_f_at | tubulin beta 2 | −1.31 | | | 1.45 | | |
| 709_at | tubulin beta 3 | −1.18 | | | | −1.35 | 1.20 |
| 471_f_at | tubulin beta 4 | −1.38 | | | 1.50 | | |
| 39399_at | tubulin beta, cofactor D | −1.85 | | | | −4.69 | |
| 32098_at | type VI collagen alpha 2 chain | | | | | | −3.79 |
| | precursor | | | | | | |
| 1651_at | ubiquitin carrier protein E2-C | −3.74 | | | | | |
| 1953_at | vascular endothelial growth factor | 1.40 | | | | | |
| 36101_s_at | vascular endothelial growth factor | 1.45 | | | | | |
| 37268_at | vascular endothelial growth factor B | | | | −1.58 | | |
| 36140_at | Y box binding protein-1 | 2.30 | 1.86 | | 2.36 | −2.72 | |

In addition, the effect of PTS893 was assessed in bone.

TABLE 14
Gene-Profiling Analysis of Salmon Calcitonin and PTS893 in Bone

| GeneChip ® | | Fold Increase | |
|---|---|---|---|
| Expression Probe | | Salmon | Fold Increase |
| Set Identifier | Coding Gene | Calcitonin | PTS893 |
| 38909_at | 25-hydroxyvitamin D3 1-alpha-hydroxylase | | −1.14 |
| 32714_s_at | activin A receptor type II-like 1 | −1.62 | |
| 35915_at | activin beta-C chain. | −1.21 | |
| 39279_at | activin type II receptor | | 1.24 |
| 39383_at | adenylate cyclase 6, isoform a | | −1.22 |
| 38965_at | aggrecan 1 | | 2.03 |
| 39206_s_at | aggrecan 1 | | 1.41 |
| 36621_at | alpha-2-HS-glycoprotein | 1.33 | |
| 34589_f_at | Amelogenin | 1.10 | −3.10 |
| 39326_at | ATPase H(+) vacuolar | −1.57 | −1.19 |
| 38814_at | ATPase H(+) vacuolar | 1.22 | |
| 33741_at | ATPase, H+ transport, lysosomal | 1.23 | |
| 33033_at | ATPase, H+ transporting, lysosomal | −1.29 | −1.17 |
| 40328_at | bHLH transcription factor | | 2.57 |
| 39407_at | bone morphogenetic protein 1 | | 1.16 |
| 31399_at | bone morphogenetic protein 10 | 1.44 | 1.20 |
| 1113_at | bone morphogenetic protein 2A | −1.12 | −1.13 |
| 40367_at | bone morphogenetic protein 2A | | −1.18 |
| 1114_at | bone morphogenetic protein 2B or BMP4 | | −1.70 |
| 1831_at | bone morphogenetic protein 5 | −1.43 | −1.60 |
| 1733_at | bone morphogenetic protein 6 precursor | | 1.27 |
| 40333_at | bone morphogenetic protein-4 (hBMP-4) | | −1.42 |
| 34847_s_at | calcium/calmodulin-dependent protein kinase (CaM kinase) II | | 1.13 |
| | beta | | |
| 33935_at | calcyclin binding protein | | 1.41 |
| 1751_g_at | Calreticulin | −4.03 | |
| 32067_at | cAMP responsive element modulator (CREM) | 1.39 | 2.75 |
| 39241_at | carbonic anhydrase I | −2.68 | |
| 40095_at | carbonic anhydrase II | −1.69 | |
| 40163_r_at | cartilage oligomeric matrix protein precursor | 2.36 | |
| 128_at | cathepsin k | 1.18 | |
| 129_g_at | cathepsin k | 1.20 | |
| 38466_at | cathepsin k | 1.27 | |
| 40718_at | cathepsin w | −1.31 | |
| 32833_at | CDC-like kinase 1 | 1.63 | |
| 646_s_at | CDC-like kinase 2 isoform hclk2/139 | 1.19 | |
| 34763_at | chondroitin sulphate proteoglycan 6 | | −1.18 |
| 598_at | collagen type II alpha-1 | −1.38 | −1.19 |
| 32488_at | collagen type III alpha 1 | −1.41 | |
| 38952_s_at | collagen type IV alpha-2 | 1.23 | 1.44 |
| 35379_at | collagen type IX alpha 1 | −2.22 | |
| 34802_at | collagen type VI alpha-2 (AA 570-998) | −1.37 | |
| 38566_at | collagen type X alpha-1 | | 1.67 |
| 37892_at | collagen type XI alpha-1 | 1.24 | 1.18 |
| 1026_s_at | collagen type XI alpha2 | −1.20 | |
| 1027_at | collagen type XI alpha2 | 1.11 | |
| 39632_at | collagenase 3 (matrix metalloproteinase 13) | 1.20 | |
| 36638_at | connective tissue growth factor. | | −1.32 |
| 1943_at | cyclin A | | −74 |
| 40697_at | cyclin A2 | −1.60 | −1.39 |
| 34736_at | cyclin B1 | −2.83 | |
| 39251_at | cyclin C | | −2.03 |
| 1983_at | cyclin D2 | | −1.28 |
| 36650_at | cyclin D2 | 1.21 | |
| 35249_at | cyclin E2 | −2.95 | |
| 1649_at | cyclin G1 interacting protein | | 1.31 |
| 1913_at | cyclin G2 | | −1.29 |
| 160024_at | cyclin-dependent kinase (CDC2-like) 10 PISSLRE | | 1.53 |
| 1942_s_at | cyclin-dependent kinase 4 | | −1.22 |
| 1206_at | cyclin-dependent kinase 5 | 1.56 | |
| 40549_at | cyclin-dependent kinase 5 | | −1.40 |
| 799_at | cyclin-dependent kinase 5, regulatory subunit 1 (p35) | 1.32 | |
| 41546_at | cyclin-dependent kinase 6 | 1.15 | |
| 2031_s_at | cyclin-dependent kinase inhibitor 1A (p21, Cip1) | 1.95 | |
| 1787_at | cyclin-dependent kinase inhibitor 1C | | 1.18 |
| 38673_s_at | cyclin-dependent kinase inhibitor 1C | | 1.13 |
| 39545_at | cyclin-dependent kinase inhibitor 1C | | 1.24 |
| 1797_at | cyclin-dependent kinase inhibitor 2D (p19, inhibits CDK4) | | −1.21 |
| 35816_at | cystatin B (stefln B) | 1.57 | |
| 806_at | cytokine-inducible kinase | 1.20 | |
| 40049_at | death-associated protein kinase 1 | | −1.30 |
| 33903_at | death-associated protein kinase 3 | −1.22 | −7.73 |
| 34029_at | dentin matrix acidic phosphoprotein 1 (DMP1) | 1.65 | |
| 38059_g_at | dermatopontin | | 1.72 |
| 343_s_at | ectonucleotide pyrophosphatase/phosphodiesterase 1 | 1.11 | |
| 342_at | ectonucleotide Pyrophosphatase/Phosphodiesterase 1 | 1.45 | |
| 1442_at | oestrogen receptor | 1.47 | |
| 33670_at | oestrogen receptor | 1.30 | |
| 1487_at | oestrogen receptor-related protein | 1.11 | |
| 38882_r_at | oestrogen-responsive B box protein (EBBP) | 1.22 | |
| 38902_r_at | oestrogen-responsive B box protein (EBBP) | | 1.23 |
| 39945_at | fibroblast activation protein | −1.27 | |
| 424_s_at | fibroblast growth factor receptor. | −1.17 | |
| 466_at | general transcription factor II, | | 1.34 |
| 1102_s_at | glucocorticoid receptor alpha | | 1.43 |
| 33510_s_at | glutamate receptor, metabotropic 1 | 1.26 | 1.23 |
| 33269_at | GPI1 N-acetylglucosaminyl transferase component Gpil | 1.24 | 1.21 |
| 41476_at | G-protein alpha subunit 11 | | 1.24 |
| 1401_g_at | granulocyte-macrophage colony-stimulating factor (CSF1) | −3.07 | −2.57 |
| 1911_s_at | growth arrest and DNA-damage-inducible protein (gadd45) | | 2.87 |
| 888_s_at | growth differentiation factor 1 | | −1.43 |
| 37615_at | growth factor receptor-bound protein 10 | 1.21 | |
| 33929_at | heparan sulphate proteoglycan (glypican). | | 2.00 |
| 39757_at | heparan sulphate proteoglycan core protein | | 1.10 |
| 755_at | inositol 1,4,5-trisphosphate receptor type 1 | | 1.27 |
| 33506_at | inositol polyphosphate 4-phosphatase type I-beta | 1.12 | −1.24 |
| 33290_at | inositol polyphosphate 5-phosphatase (5ptase) | | −1.20 |
| 32697_at | inositol(myo)-1(or 4)-monophosphatase 1 | −1.36 | |
| 1975_s_at | insulin-like growth factor 1 | | −1.41 |
| 1501_at | insulin-like growth factor 1 (somatomedin C) | | −1.12 |
| 1232_s_at | insulin-like growth factor binding protein | −1.31 | |
| 40422_at | insulin-like growth factor binding protein 2 | | −1.27 |
| 1586_at | insulin-like growth factor binding protein 3 | 1.45 | |
| 37319_at | insulin-like growth factor binding protein 3 | 2.17 | |
| 1737_s_at | insulin-like growth factor binding protein 4 | | 1.13 |
| 41420_at | insulin-like growth factor binding protein 5 | | 1.18 |
| 1396_at | insulin-like growth factor binding protein 5 | | 1.62 |
| 1678_g_at | insulin-like growth factor binding protein 5 | | 1.44 |
| 38650_at | insulin-like growth factor binding protein 5 | | 1.53 |
| 1741_s_at | insulin-like growth factor binding protein-2 | −2.49 | −2.11 |
| 1464_at | insulin-like growth factor II precursor | 1.18 | |
| 1591_s_at | insulin-like growth factorn II precursor | 1.41 | 1.31 |
| 39781_at | insulin-like growth factor-binding protein 4 | | 1.16 |
| 33082_at | integrin alpha 10 subunit | 1.33 | |
| 35131_at | integrin-binding sialoprotein (bone sialoprotein, bone | | 1.15 |
| | sialoprotein II) | | |
| 40060_r_at | LIM protein (similar to rat protein kinase C-binding enigma) | 1.44 | 1.32 |
| 36184_at | lysyl hydroxylase (PLOD) procollagen-lysine, 2-oxoglutarate 5 | | −1.40 |
| | dioxygenase | | |
| 34795_at | lysyl hydroxylase isoform 2 (PLOD2) | | 1.49 |
| 36811_at | lysyl oxidase-like protein | −1.44 | |
| 1433_g_at | MAD, mothers against decapentaplegic homolog 3 | 1.14 | 1.73 |
| 34655_at | MAGUKs (membrane-associated guanylate kinase homologues | 1.23 | |
| 36179_at | MAP kinase activated protein kinase 2 | | 1.18 |
| 35652_g_at | MAP kinase kinase kinase (MTK1) | 1.14 | |
| 41279_f_at | MAPK8IP1 Mitogen-activated protein kinase 8 interacting | | 1.25 |
| | protein 1 | | |
| 41280_r_at | MAPK8IP1: mitogen-activated protein kinase 8 interacting | −1.31 | −1.31 |
| | protein 1 | | |
| 1509_at | Metalloproteinase | −1.42 | |
| 976_s_at | mitogen-activated protein kinase 1 | −1.61 | 1.12 |
| 34006_s_at | mitogen-activated protein kinase 8 | 1.32 | |
| 1439_s_at | mitogen-activated protein kinase-activated protein kinase 2 | | 1.78 |
| 37565_at | MMD: monocyte to macrophage differentiation-associated | 1.28 | 1.30 |
| 38369_at | myeloid differentiation primary response gene (88) | | −1.10 |
| 1052_s_at | NF-IL6-beta protein | | 1.30 |
| 36472_at | N-myc and STAT interacter- | | −1.35 |
| 38354_at | nuclear factor NF-IL6 (AA 1-345) | | 1.92 |
| 33106_at | nuclear orphan receptor LXR-alpha nuclear receptor subfamily | | 3.29 |
| | 1, group H, member 3 | | |
| 33381_at | nuclear receptor co-activator | | 1.11 |
| 279_at | nuclear receptor subfamily 4, group A, member 1 | | 2.30 |
| 280_g_at | nuclear receptor subfamily 4, group A, member 1 | | 3.08 |
| 37623_at | nuclear receptor subfamily 4, group A, member 2 Member of the | | 27.72 |
| | steroid/thyroid hormone receptor family | | |
| 547_s_at | nuclear receptor subfamily 4, group A, member 2 Member of the | | 26.77 |
| | steroid/thyroid hormone receptor family | | |
| 190_at | nuclear receptor subfamily 4, group A, member 3 Member of | | 5.45 |
| | steroid/thyroid receptor family of nuclear hormone receptors | | |
| 41202_s_at | OS-4 protein (OS-4) | 1.24 | |
| 1451_s_at | OSF-2os osteoblast specific factor-2 (periostin) | −1.65 | |
| 38822_at | O-sialoglycoprotein endopeptidase | | 2.43 |
| 467_at | osteoclast stimulating factor (OSF | −1.23 | |
| 35107_at | osteoprotegerin ligand | | 3.33 |
| 33814_at | PAK4 protein | 1.16 | |
| 38757_at | PDGF associated protein. | −1.89 | |
| 40253_at | phosphatidylinositol 4-kinase (NPIK-C). | 1.77 | |
| 37412_at | phosphatidylinositol-4-phosphate 5-kinase isoform C (−1) | −1.87 | |
| 751_at | phosphatidylinositol-glycan-class C (PIG-C) | 1.14 | −1.25 |
| 666_at | phosphodiesterase 4A, cAMP-specific | 1.33 | 1.30 |
| 38526_at | phosphodiesterase 4D, cAMP-specific | 1.30 | 3.53 |
| 38921_at | phosphodiesterase IB, calmodulin-dependent | 1.52 | |
| 38944_at | phosphodiesterase IB, calmodulin-dependent | | 1.17 |
| 32029_at | phosphoinositide dependent protein kinase-1 (3) | | 1.16 |
| 31699_at | phosphoinositide-3-kinase | 1.56 | 1.16 |
| 1085_s_at | phospholipase C | | −1.14 |
| 364_s_at | phospholipase C b3 | 1.22 | |
| 901_g_at | phospholipase C, beta 4 | −1.20 | |
| 1293_s_at | phospholipase D | −1.26 | |
| 32306_g_at | preprocollagen type I alpha-2 | 1.19 | |
| 35473_at | preprocollagen type I alpha 1. | −2.72 | |
| 38951_at | PRKCQ Protein kinase C, theta | | 1.43 |
| 32307_s_at | procollagen | 1.13 | |
| 34494_at | procollagen I-N proteinase. | | 1.92 |
| 37605_at | procollagen type II alpha 1 | | 1.91 |
| 36109_at | prolidase (imidodipeptidase) PEPD | −2.55 | |
| 1884_s_at | proliferating cell nuclear antigen | −1.85 | |
| 34390_at | prolyl 4-hydroxylase alpha (II) subunit | | 1.19 |
| 37037_at | prolyl 4-hydroxylase alpha subunit | | 1.20 |
| 36666_at | prolyl 4-hydroxylase beta | 1.95 | |
| 36533_at | prostacyclin synthase | | 1.20 |
| 718_at | protease, serine, 11 (IGF binding) | −1.30 | |
| 719_g_at | protease, serine, 11 (IGF binding) | −1.43 | |
| 385_at | proteasome (prosome, macropain) subunit, beta type, 10 | 1.36 | |
| 39183_at | protein kinase 1 PCTAIRE | −1.17 | |
| 37698_at | protein kinase A (PRKA) anchor protein 1 | | 1.29 |
| 39711_at | protein kinase C substrate 80K-H | | 1.13 |
| 39161_at | protein kinase Njmu-R1 | | 1.21 |
| 35348_at | protein kinase, AMP-activated, beta 1 non-catalytic subunit | | 2.10 |
| 36359_at | protein kinase, cAMP-dependent, catalytic, gamma | 1.39 | |
| 546_at | protein kinase, cAMP-dependent, catalytic, inhibitor alpha | | 1.14 |
| 227_g_at | protein kinase, cAMP-dependent, regulatory, type I, alpha | | 1.18 |
| 41768_at | protein kinase, cAMP-dependent, regulatory, type I, alpha | | 1.15 |
| 1091_at | protein kinase, cAMP-dependent, regulatory, type I, beta | 1.65 | |
| 116_at | protein kinase, cAMP-dependent, regulatory, type II, alpha | 1.28 | |
| 33633_at | purinergic receptor P2Y, G-protein coupled, 11 | 1.90 | |
| 32737_at | RAC2 Ras-related C3 botulinum toxin substrate 2 (rho family, | 1.16 | |
| | small GTP binding protein Rac2) | | |
| 40299_at | RE2 G-protein coupled receptor | | 1.24 |
| 35668_at | receptor (calcitonin) activity modifying protein 1 RAMP 1 | | 1.34 |
| 40696_at | receptor (TNFRSF)-interacting serine-threonine kinase 1 | | 1.12 |
| 1007_s_at | receptor tyrosine kinase DDR | 1.21 | |
| 37701_at | regulator of G-protein signalling 2, 24 kD | | 2.06 |
| 1048_at | retinoid X receptor-gamma | 1.47 | 1.34 |
| 36217_at | serine/threonine kinase 38 | 1.54 | |
| 41544_at | serum-inducible kinase | | 1.16 |
| 32447_at | SF-1; Steroidogenic factor-1 | 8.76 | |
| 36487_at | short stature homeobox 2, | | −1.46 |
| 41222_at | signal transducer and activator of transcription 6 (STAT6) | 1.44 | |
| 1955_s_at | SMAD6 (inhibits BMP/Smad1 (MADH1) signalling) | 1.19 | |
| 37718_at | SNF-1 related kinase | 1.49 | 1.19 |
| 35883_at | Spi-B | | −2.80 |
| 1244_at | Stat2 | | −1.12 |
| 506_s_at | Stat5A | | 1.16 |
| 38994_at | STAT-induced STAT inhibitor-2 | | 1.25 |
| 38669_at | Ste20-related serine/threonine kinase | 1.24 | 1.65 |
| 37152_at | steroid hormone receptor superfamily | | 1.19 |
| 35844_at | syndecan 4 | | 1.37 |
| 38374_at | TEIG; TGFB inducible early growth response | 1.18 | |
| 38427_at | TEIG; TGFB inducible early growth response | | 1.38 |
| 32080_at | tetracycline transporter-like protein | | 1.41 |
| 224_at | TGFB inducible early growth response; TIEG | 1.26 | |
| 36940_at | TGFB1-induced anti-apoptotic factor 1 | 1.22 | 1.60 |
| 32217_at | TGF-beta induced apotosis protein 12 | 1.40 | |
| 41445_at | TGF-beta precursor | 1.14 | |
| 1890_at | TGF-beta superfamily protein | 1.74 | |
| 40631_at | Tob | −1.14 | 1.59 |
| 39358_at | transcriptional co-repressor nuclear receptor co-repressor 2 | | 1.42 |
| 1385_at | transforming growth factor induced protein | | 1.36 |
| 1830_s_at | transforming growth factor-beta | | 1.17 |
| 1767_s_at | transforming growth factor-beta 3 (TGF-beta 3) | −1.71 | −1.63 |
| 40581_at | TRIO: triple functional domain (PTPRF interacting) | 1.65 | 1.56 |
| 32272_at | tubulin alpha | −1.20 | |
| 685_f_at | tubulin alpha isotype H2-alpha | −4.36 | −1.79 |
| 330_s_at | tubulin alpha, 1, | −1.80 | −1.15 |
| 151_s_at | tubulin beta | −1.40 | |
| 39399_at | tubulin beta cofactor D | −1.85 | |
| 471_f_at | tubulin beta, 4 | −1.38 | |
| 40567_at | tubulin, alpha 3 | −1.39 | |
| 709_at | tubulin, beta 3 | −1.18 | |
| 33678_i_at | tubulin, beta, 2 | −1.15 | |
| 33679_f_at | tubulin, beta, 2 | −1.31 | |
| 1651_at | ubiquitin carrier protein E2-C | −3.74 | −1.22 |
| 32548_at | unactive progesterone receptor | | −1.33 |
| 1953_at | vascular endothelial growth factor | 1.40 | 1.20 |
| 36101_s_at | vascular endothelial growth factor | 1.45 | 1.44 |
| 36140_at | Y box binding protein-1 | 2.30 | 5.49 |

− numbers = fold down-regulated
+ numbers = fold up-regulated

Real-time PCR. Based on the DNA microarray data a set of transcripts was chosen for quantitative analysis by real time-PCR (RT-PCR).

Briefly, the method exploits the SyBr Green dye which intercalates into double stranded DNA. Accumulation of PCR products is detected directly by monitoring the increase in fluorescence of the SyBr Green dye. Reactions are characterised by the point in time during cycling when amplification of a PCR product is first detected rather than the amount of PCR product accumulated after a fixed number of cycles. The higher the starting copy number of nucleic acid target, the sooner a significant increase in fluorescence is observed.

From each RNA sample, cDNA was made using an Applied Biosystem kit (Applied Biosystems # N808-0234) following the recommendation of the manufacturer. The PCR mixture was prepared using the SyBr Green Universal PCR Master Mix (Applied Biosystems # 4309155) as follows: 5 μl cDNA template, 400 nM of each primer, 0.2 mM deoxynucleotide triphosphates, 1 mM MgCl2 and 0.5 U Taq DNA polymerase, 5 μl SyBr Green PCR buffer and RNase free water up to a final volume of 50 μl. The PCR was performed using the ABI Prism 7700 Sequence Detection System, after a step at 95° C. for 10 min, the step-cycle program was performed for a total of 40 cycles as follows: 95° C. for 30 s, 60° C. for 1 min. A negative control was included: PCR reaction mixture with water in place of the cDNA sample.

The initial template concentration was determined based on the threshold cycle. The threshold cycle is the PCR cycle at which fluorescence is first detected above background and has been shown to be inversely proportional to the number of target copies present in the sample. Quantification was performed by calculating the unknown target concentration relative to an absolute standard and by normalizing to a validated endogenous control such as a housekeeping gene (β-actin). Results are presented as percentage of control, once the ratio between the numbers of molecule for the gene of interest divided by the number of molecule for beta-actin has been calculated.

Based on the DNA microarray data the following set of transcripts was chosen for quantitative analysis by RT-PCR: adhesion receptor CD44, angiopoietin, bone morphogenetic protein 5, carbonic anhydrase II, cartilage oligomeric matrix protein, cathepsin K, osteopontin, pre-pro-alpha-2 type I collagen, Spi-B and Y-box binding protein.

TABLE 15
Real Time PCR Results

| GeneChip ® | | Treatment Effect | Treatment Effect |
|---|---|---|---|
| Expression Probe | | Salmon Calcitonin | PTS893 |
| Set Identifier | Coding Gene | (% respect to control) | (% respect to control) |
| 1372_at | adhesion receptor | No change | No change |
| | CD44 | | |
| 1929_at | angiopoietin-1 | No change | No change |
| 1831_at | bone morphogenetic | +16 | +18 |
| | protein 5 | | |
| 40095_at | carbonic anhydrase II | −60 | No change |
| 40161_at | cartilage oligomeric | +34.23 | No change |
| | matrix protein | | |
| 128_at | cathepsin K | +67.2 | No change |
| 2092_s_at | osteopontin | No change | No change |
| 32306_g_at | pre-pro-alpha-2 type I | +38 | +62 |
| | collagen | | |
| 35883_at | Spi-B | −44 | −18 |
| 36140_at | Y-box binding | +14 | +26 |
| | protein (bone) | | |
| 36140_at | Y-box binding | +15 | n.a. |
| | protein (kidney) | | |
| 36140_at | Y-box binding | −26 | n.a. |
| | protein (muscle) | | |

n.a.: not applicable

RT-PCR confirmed in most of the cases the changes observed in the gene profiling analysis, as it was the case for bone morphogenetic protein 5, carbonic anhydrase II, cathepsin K, cartilage oligomeric matrix protein, pre-pro-alpha-2 type I collagen, Spi-B and Y-Box binding protein. No changes were however detected in the level of expression of adhesion receptor CD44, angiopoietin-1 and osteopontin.

Analysis. Calcitonin is known to exert an effect on the differentiation, survival and resorptive activity of osteoclasts, resulting in a decreased osteoclastic activity. Pondel M, Intl. J. Exp. Pathol. 81(6): 405-22 (2000). These effects could be reconstructed by multi-organ gene profiling (TABLE 16).

TABLE 16
Effects on Osteoclasts

| | | Salmon | |
|---|---|---|---|
| Function | Coding genes | Calcitonin | PTS893 |
| Osteoclast | PU.1 (SPI1) | B, K, P, T | B |
| determination. | Granulocyte to macrophage | B, K | B |
| survival and | colony-stimulating factor (CSF1) | | |
| differentiation | Monocyte to macrophage | B, K, T | B |
| | differentiation associate (MMD) | | |
| | Osteoclast stimulating factor 1 | B, K, L, P | |
| | (Autocrine stimulation of | | |
| | osteoclast resorptive activity) | | |
| Bone resorption | H+ ATP-ases | ALL | B |
| by osteoclast | Carbonic anhydrase I, II. | B, L, P | |
| | Cathepsin K | ALL | |
| | ODF/OPGL: osteoprotegerin | | B |
| | ligand | | |
| Osteoclast | Tubulins | ALL | |
| motility | PAK4 protein | B, M, P | |

Multi-organ gene expression profiling in salmon calcitonin treated animals. Organs where changes in expression were seen are displayed.
B = bone;
K = kidney;
M = muscle;
P = pituitary;
L = liver;
T = trachea.

Salmon calcitonin seems to exert a paracrine regulation of the osteoclast resorptive activity, through the regulation of cystatin expression in the osteoblast, as shown in TABLE 17.

TABLE 17
Gene Expression Profiling: Osteoclast Function

| GeneChip ® | | | | |
|---|---|---|---|---|
| expression | | | | |
| probe set | | Average | Average | Fold |
| identifier | Coding Gene | controls | sCT | change |
| 40729_s_at | ATPase, H+ transporting, | 204 | 327 | 1.6 |
| | lysosomal (vacuolar proton | | | |
| | pump) subunit G isoform 2 | | | |
| 37367_at | ATPase, H+ transporting, | 272 | 328 | 1.2 |
| | lysosomal 31 kDa, V1 | | | |
| | subunit E isoform 1 | | | |
| 40568_at | ATPase, H+ transporting, | 938 | 1132 | 1.21 |
| | lysosomal 56/58 kDa, V1 | | | |
| | subunit B, isoform 2 | | | |
| 39241_at | carbonic anhydrase I | 1266 | 441 | −2.87 |
| 128_at | cathepsin K (pycnodysostosis) | 5690 | 7821 | 1.37 |
| 129_g_at | cathepsin K (pycnodysostosis) | 5036 | 6757 | 1.34 |
| 38466_at | cathepsin K (pycnodysostosis) | 5494 | 7267 | 1.32 |
| 36611_at | acid phosphatase 1, soluble | 254 | 331 | 1.3 |

PU.1 is involved in the initial stages of osteoclastogenesis. Tondravi M M et al., Nature 386(6620): 81-4 (1997). CSF-1 is imperative for macrophage maturation; it binds to its receptor c-fms on early osteoclast precursors, providing signals required for their survival and proliferation. Teitelbaum S L, Science 289(5484):1504-1508 (2000).

Interestingly, PTS893 also regulates the genes implicated in osteoclast differentiation and survival, SPI1, CSF-1 and MMD. This osteoclast regulation has not been previously described.

Salmon calcitonin was shown to regulate the expression of the gene coding for osteoclast stimulating factor (OSF), which is an intracellular protein produced by osteoclasts that indirectly induces osteoclast formation and bone resorption. Reddy S et al., J. Cell Physiol. 177 (4): 636-45 (1998). This would imply an autocrine effect of salmon calcitonin in the regulation of the osteoclast function, which is described here for the first time.

In addition, salmon calcitonin seems to exert a paracrine regulation of the osteoclast resorptive activity, through the regulation of cystatin expression in the osteoblast. Carbonic anhydrase I, II, H⁺-ATPases and cathepsin K are the main effectors for dissolving bone mineral and matrix degradation. Blair H C et al., Biochem. (2002). Regulation of tubulins and PAK4 genes can be related to the effect of calcitonin on osteoclast motility PAK 4. Zaidi M et al., Bone 30(5): 655-63 (2002); Jaffer Z M & Chemoff J, Intl. J. Biochem. Cell Biol. 34(7): 713-7 (2002).

These results show modulating effects of calcitonin on genes affecting the direct, autocrine, paracrine and endocrine regulation of the osteoblast function (TABLE 18). These data support the hypothesis that attributes a bone anabolic effect to calcitonin.

TABLE 18
Effects on Osteoblasts

| | | Salmon | |
|---|---|---|---|
| Function | Coding Gene | Calcitonin | PTS893 |
| Antagonists of | Cystatins | B | |
| cathepsins: | Alpha-2-HS-Glycoprotein | B, K, T | |
| antiresorptive activity | Bone Morphogenetic Proteins | ALL | B |
| Autocrine/paracrine | Fibroblast Growth Factors | B, K, M, P, T | B |
| regulation of | IL6/LIF | | B |
| osteoblast function | Insulin-like Growth Factors | ALL | B |
| | TGFs | B, K, M, P | B |
| | Tob | B, M, P | B |
| | Vascular Endothelial Growth Factor | B, M | X |
| Endocrine regulation | Activin | B, L, M, P | B |
| of osteoblast function | Oestrogen receptor | ALL | |
| | Retinoic receptor X | B, P | B |
| | Steroidogenic factor | B, L, P, T | |
| | nuclear receptors (steroid/thyroid | | B |
| | family) | | |
| Transcription factor | Y-box binding protein | B, K, M, P | B |
| that regulates collagen | | | |
| type 1 synthesis | | | |

Multi-organ gene expression profiling in salmon calcitonin treated animals. Organs where changes in expression were seen are displayed.
B = bone;
K = kidney;
M = muscle;
P = pituitary;
L = liver;
T = trachea.

The results of this EXAMPLE show modulating effects of calcitonin on genes affecting the direct, autocrine, paracrine and endocrine regulation of the osteoblast function. These data support the hypothesis that attributes a bone anabolic effect to calcitonin.

Three families of growth factors, the transforming growth factor betas (TGF-βs), insulin-like growth factors (IGFs), and bone morphogenetic proteins (BMPs), are considered to be principal local regulators of osteogenesis. Bone morphogenetic proteins are thought to have their major effects on early precursor bone cell replication and osteoblast commitment. In contrast, TGB-βs are thought to be the most potent inducers of committed bone cell replication and osteoblast matrix production, while IGFs appear to integrate and extend the effect of both factors. McCarthy T L et al., Crit Rev. Oral Biol. Med. 11(4): 409-22 (2000). These results support the fact that both salnon calcitonin and PTS893 are able to regulate these local and systemic factors implicated in bone metabolism.

The fact that salmon calcitonin regulates α2-HS glycoprotein (AHSG), which blocks TGF-β-dependent signalling in osteoblastic cells, also supports this role. Mice lacking AHSG display growth plate defects, increased bone formation with age, and enhanced cytokine-dependent osteogenesis. Szweras M et al., J. Biol. Chem., 277(22): 19991-19997 (2002).

Salmon calcitonin and PTS893 were also shown to modulate the expression of the genes coding for vascular endothelial growth factor (VEGF). VEGF is known for playing a key role in normal and pathological angiogenesis. The critical role of angiogenesis for successful osteogenesis during the endochondral ossification is well documented. VEGF indirectly induces proliferation and differentiation of osteoblasts by stimulating endothelial cells to produce osteoanabolic growth factors. Wang D S et al., Endocrinology 138(7): 2953-62 (1997). In addition, VEGF stimulates chemotactic migration of primary human osteoblasts, suggesting a functional role in bone formation and remodelling. Mayr-Wohlfahrt U et al., Bone 30 (3): 472-7 (2002).

The effects of parathyroid hormone on osteoblast for mediating both bone resorption and formation have been widely described. Swarthout J T et al., Gene 282(1-2):1-17 (2002). It was here possible to confirm the effect of PTS893 on cytokines like interleukin 6 (IL-6), which mediates the paracrine activation of osteoclast differentiation and activity. Greenfield E M et al., Life Sci. 65:1087-102 (1999). PTS893 also produced a strong up-regulation on nuclear receptors (steroid/thyroid family).

TABLE 19
Gene Expression Profiling: Growth Factors and Hormones

| GeneChip ® | | | | |
|---|---|---|---|---|
| expression | | | | |
| probe set | | Average | | |
| identifier | Coding Gene | controls | Average sCT | Fold change |
| 39407_at | bone morphogenetic protein 1 | 448 | 607 | 1.36 |
| 1122_f_at | chorionic gonadotropin, beta polypeptide | 263 | 380 | 1.44 |
| 39945_at | fibroblast activation protein, alpha | 636 | 436 | −1.46 |
| 1970_s_at | fibroblast growth factor receptor 2 | 184 | 108 | −1.69 |
| | (bacteria-expressed kinase, keratinocyte | | | |
| | growth factor receptor, craniofacial | | | |
| | dysostosis 1, Crouzon syndrome, Pfeiffer | | | |
| | syndrome, Jackson-Weiss syndrome) | | | |
| 32254_at | follistatin-like 3 (secreted glycoprotein) | 1514 | 2209 | 1.46 |
| 38737_at | insulin-like growth factor 1 (somatomedin | 66 | 37 | −1.79 |
| | C) | | | |
| 36782_s_at | insulin-like growth factor 2 (somatomedin | 212 | 323 | 1.52 |
| | A) | | | |
| 1591_s_at | insulin-like growth factor 2 (somatomedin | 293 | 402 | 1.37 |
| | A) | | | |
| 40422_at | insulin-like growth factor binding protein | 181 | 105 | −1.73 |
| | 2, 36 kDa | | | |
| 37319_at | insulin-like growth factor binding protein 3 | 495 | 1561 | 3.15 |
| 1586_at | insulin-like growth factor binding protein 3 | 428 | 722 | 1.69 |
| 37319_at | insulin-like growth factor binding protein 3 | 604 | 879 | 1.46 |
| 1586_at | insulin-like growth factor binding protein 3 | 355 | 445 | 1.25 |
| 1451_s_at | osteoblast specific factor 2 (fasciclin I- | 538 | 292 | −1.84 |
| | like) periostin | | | |
| 532_at | parathyroid hormone receptor 1 | 1337 | 1849 | 1.38 |
| 234_s_at | pleiotrophin (osteoblast specific factor 1) | 710 | 507 | −1.4 |
| 34820_at | pleiotrophin (heparin binding growth | 422 | 329 | −1.28 |
| | factor 8, neurite growth-promoting factor | | | |
| | 1) | | | |
| 1897_at | transforming growth factor beta 1 induced | 176 | 296 | 1.68 |
| | transcript 1 | | | |
| 1385_at | transforming growth factor, beta-induced, | 187 | 292 | 1.57 |
| | 68 kDa | | | |
| 39588_at | tumour necrosis factor (ligand) | 176 | 127 | −1.39 |
| | superfamily, member 12 | | | |
| 31410_at | tumour necrosis factor (ligand) | 197 | 128 | −1.54 |
| | superfamily, member 4 | | | |
| 38631_at | tumour necrosis factor receptor | 134 | 240 | 1.79 |
| | superfamily, member 13B | | | |
| 35150_at | tumour necrosis factor receptor | 443 | 298 | −1.48 |
| | superfamily, member 5 | | | |
| 595_at | tumour necrosis factor, alpha-induced | 118 | 191 | 1.62 |
| | protein 3 | | | |
| 1953_at | vascular endothelial growth factor | 351 | 557 | 1.59 |
| 36100_at | vascular endothelial growth factor | 282 | 407 | 1.45 |
| 1953_at | vascular endothelial growth factor | 521 | 629 | 1.21 |
| 37268_at | vascular endothelial growth factor B | 379 | 504 | 1.33 |
| 39091_at | vitamin A responsive; cytoskeleton related | 421 | 299 | −1.41 |

Both calcitonin and parathyroid hormone receptors belong to the G-protein receptor superfamily. After receptor stimulation, signal transduction is mediated by adenylate cyclase/cAMP/protein kinase, Phospholipase C, Phospholipase D, and MAPK (as a late effecter) pathways in the case of calcitonin, and by adenylate cyclase and phospholipase C in the case of parathyroid hormone. Gene profiling analysis allowed the reconstruction of these pathways, showing genes that were modulated by the treatment and that are localised at different levels of the signal transduction pathway.

TABLE 20
Effects on Signal Transduction and Cell Cycle

| | | Salmon | |
|---|---|---|---|
| Function | Coding Gene | Calcitonin | PTS893 |
| Signal | Adenylate cyclase | | B |
| transduction. | Calcyclin binding protein | | B |
| | Calreticulin | B, K, M | |
| | CREM | B, L, P | B |
| | CDC Kinase | B, M | |
| | MAPK | ALL | B |
| | Protein kinases | ALL | |
| | Phosphatidylinositol pathway | ALL | B |
| | Phosphodiesterase (IB, 4A, 4B) | ALL | B |
| | Phospholipase (C, D) | ALL | B |
| | PCNA | B | |
| | SMAD pathway | ALL | B |
| | STAT pathway | ALL | B |
| Cell cycle | Cyclins (A, A2, B1, C, D2, | B | B |
| | E2, G1, G2) | | |
| | Cyclin-dependent kinases 5, 6, 10 | B, K, P, T | B |
| | Cyclin-dependent kinases | B | B |
| | inhibitor 1A, 1C, 2D) | | |

Multi-organ gene expression profiling in salmon calcitonin treated animals. Organs where changes in expression were seen are displayed.
B = bone;
K = kidney;
M = muscle;
P = pituitary;
L = liver;
T = trachea.

Salmon calcitonin seems also to exert a direct influence on cell cycle, since changes in cyclins and cyclin-related proteins could be also observed, as shown in TABLE 21.

TABLE 21
Gene Expression Profiling: Signal Transduction

| GeneChip ® | | | | |
|---|---|---|---|---|
| expression | | | | |
| probe set | | Average | Average | Fold |
| identifier | Coding Gene | controls | sCT | change |
| 769_s_at | annexin A2 | 8393 | 6969 | −1.2 |
| 32066_g_at | cAMP responsive element | 168 | 231 | 1.38 |
| | modulator | | | |
| 40777_at | catenin (cadherin-associated | 3688 | 4689 | 1.27 |
| | protein), beta 1, 88 kDa | | | |
| 40697_at | cyclin A2 | 212 | 128 | −1.65 |
| 40697_at | cyclin A2 | 272 | 175 | −1.56 |
| 1943_at | cyclin A2 | 121 | 83 | −1.45 |
| 2020_at | cyclin D1 (PRAD1: | 238 | 135 | −1.76 |
| | parathyroid adenomatosis 1) | | | |
| 36650_at | cyclin D2 | 204 | 312 | 1.53 |
| 40225_at | cyclin G associated kinase | 827 | 1011 | 1.22 |
| 31700_at | G protein-coupled receptor 35 | 844 | 591 | −1.43 |
| 41074_at | G protein-coupled receptor 49 | 242 | 146 | −1.66 |
| 33082_at | integrin, alpha 10 | 171 | 243 | 1.42 |
| 33082_at | integrin, alpha 10 | 228 | 289 | 1.26 |
| 33411_g_at | integrin, alpha 6 | 65 | 35 | −1.86 |
| 33410_at | integrin, alpha 6 | 201 | 90 | −2.22 |
| 38297_at | phosphatidylinositol transfer | 753 | 1006 | 1.34 |
| | protein, membrane-associated | | | |
| 31904_at | phosphodiesterase 2A, cGMP- | 555 | 740 | 1.33 |
| | stimulated | | | |
| 38269_at | protein kinase D2 | 747 | 1067 | 1.43 |
| 36008_at | protein tyrosine phosphatase | 518 | 376 | −1.38 |
| | type IVA, member 3 | | | |
| 35361_at | PTEN induced putative | 95 | 255 | 2.69 |
| | kinase 1 | | | |
| 35178_at | WNT inhibitory factor 1 | 1746 | 2127 | 1.22 |

Bone morphogenetic protein (BMP) controls osteoblast proliferation and differentiation through Smad proteins. Tob, a member of the emerging family of antiproliferative proteins, is a negative regulator of BMP/Smad signalling in osteoblasts. Smad pathway as well as Tob as one of their regulators were also identified as genes modulated by the sCT and PTS893 treatment, in agreement with the hypothesised effect of both compounds on BMP regulation of bone remodelling. Within this context, both compounds seem to exert a direct influence on cell cycle, since changes in cyclins and cyclin-related proteins could be also observed.

Both compounds regulate also synthesis and degradation of extracellular matrix components (TABLE 22).

TABLE 22
Effects on Extracellular Matrix

| | | Salmon | |
|---|---|---|---|
| Function | Coding Gene | Calcitonin | PTS893 |
| Cell attachment. | Integrins | B, M, P | B |
| Signal transduction. | | | |
| Collagen digestion | Collagenase | B | |
| | Matrix metalloproteinases I, II | B, L, P, T | |
| Collagen synthesis | Procollagen endopeptidase/proteinase | | B |
| | Lysyl hydroxylase | | B |
| Extracellular matrix | Aggrecan | | B |
| component | Cartilage Oligomeric Matrix Protein | B, K, | |
| | Precursor | | |
| | Collagen type I, type II, type III, type IV, | ALL | B |
| | type V, type VI, type IX, type X, type XI, | | |
| | type XIII, type XIV, type XV, and/or type XVI) | | |
| | Chondroitin sulphate proteoglycan | K, M, T | B |
| | Dermatopontin | | B |
| | Heparan sulphate proteoglycan | L, T | B |
| | Syndecan | | B |

Multi-organ gene expression profiling in salmon calcitonin-treated animals. Organs where changes in expression were seen are displayed.
B = bone;
K = kidney;
M = muscle;
P = pituitary;
L = liver;
T = trachea.

Salmon calcitonin regulates also the synthesis and degradation of extracellular matrix components, as shown in TABLE 23.

TABLE 23
Gene Expression Profiling: Extracellular Matrix

| GeneChip ® | | | | |
|---|---|---|---|---|
| expression | | | | |
| probe set | | Average | Average | Fold |
| identifier | Coding Gene | controls | sCT | change |
| 36253_at | bone gamma-carboxyglutamate (gla) | 26305 | 33265 | 1.26 |
| | protein (osteocalcin) | | | |
| 32094_at | carbohydrate (chondroitin 6) | 253 | 130 | −1.95 |
| | sulfotransferase 3 | | | |
| 32094_at | carbohydrate (chondroitin 6) | 292 | 241 | −1.21 |
| | sulfotransferase 3 | | | |
| 41447_at | carbohydrate (chondroitin) synthase 1 | 192 | 107 | −1.79 |
| 34042_at | chondroadherin | 7965 | 10266 | 1.29 |
| 32306_g_at | collagen, type I, alpha 2 | 7740 | 9337 | 1.21 |
| 32488_at | collagen, type III, alpha 1 (Ehlers-Danlos | 2399 | 1294 | −1.85 |
| | syndrome type IV, autosomal dominant) | | | |
| 34802_at | collagen, type VI, alpha 2 | 2374 | 1500 | −1.58 |
| 35816_at | cystatin B (stefin B) | 983 | 1897 | 1.93 |
| 34029_at | dentin matrix acidic phosphoprotein | 216 | 587 | 2.72 |
| 38059_g_at | dermatopontin | 695 | 962 | 1.38 |
| 38057_at | dermatopontin | 1090 | 1381 | 1.27 |
| 33929_at | glypican 1 | 235 | 163 | −1.44 |
| 39350_at | glypican 3 | 64 | 50 | −1.29 |
| 37176_at | hyaluronoglucosaminidase 1 | 109 | 266 | 2.43 |
| 1546_at | hyaluronoglucosaminidase 1 | 49 | 79 | 1.59 |
| 36683_at | matrix Gla protein | 65 | 117 | 1.8 |
| 609_f_at | metallothionein 1B (functional) | 2693 | 3485 | 1.29 |
| 870_f_at | metallothionein 3 (growth inhibitory factor | 1744 | 2296 | 1.32 |
| | (neurotrophic)) | | | |
| 38307_at | neurochondrin | 452 | 696 | 1.54 |
| 34342_s_at | secreted phosphoprotein 1 (osteopontin, | 16370 | 21279 | 1.3 |
| | bone sialoprotein I) | | | |
| 38127_at | syndecan 1 | 534 | 346 | −1.54 |
| 1693_s_at | tissue inhibitor of metalloproteinase 1 | 4549 | 5522 | 1.21 |
| | (erythroid potentiating activity, | | | |
| | collagenase inhibitor) | | | |
| 2092_s_at | secreted phosphoprotein 1 (osteopontin, | 7748 | 9576 | 1.24 |
| | bone sialoprotein I, early T-lymphocyte | | | |
| | activation 1) | | | |
| 38308_g_at | neurochondrin | 679 | 490 | −1.39 |

Of particular interest is the regulation of the Y-Box binding protein (YB-1), which appears to be modulated by both treatments and in four out of six organs analysed in the salmon calcitonin group. YB-1 is a protein that interacts with a TGF-β response element in the distal region of the collagen alpha 1(I) gene. YB-1 protein activates the collagen promoter and translocates into the nucleus during TGF-β addition to fibroblasts, suggesting a role for this protein in TGF-β signalling. Sun W et al., Matrix Biol. 20(8): 527-41 (2001).

In addition, salmon calcitonin and PTS893 regulated some aspects of the mineralization of the bone extracellular matrix, since changes in amelogenin, dentin and ectonucleotide pyrophosphatases were observed.

TABLE 20
Effects on Mineralization and Visualisation

| | | Salmon | |
|---|---|---|---|
| Function | Coding Gene | Calcitonin | PTS893 |
| Cement component | Amelogenin | B, L | B |
| Mineral matrix protein | Dentin | B | B |
| Enzyme for synthesis of | Ectonucleotide | B, M | |
| inorganic Pi | pyrophosphatases | | |
| Growth factor | VEGF | B, M | B |
| vascularisation | | | |

Multi-organ gene expression profiling in salmon calcitonin treated animals. Organs where changes in expression were seen are displayed.
B = bone;
K = kidney;
M = muscle;
P = pituitary;
L = liver;
T = trachea.

All references cited herein are incorporated herein by reference in their entirety and for all purposes to the same extent as if each individual publication or patent or patent application was specifically and individually indicated to be incorporated by reference in its entirety for all purposes. In addition, all GenBank accession numbers, Unigene Cluster numbers and protein accession numbers cited herein are incorporated herein by reference in their entirety and for all purposes to the same extent as if each such number was specifically and individually indicated to be incorporated by reference in its entirety for all purposes.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatus within the scope of the invention, in addition to those enumerated herein, will be apparent to those skilled in the art from the foregoing description and accompanying drawings. Such modifications and variations are intended to fall within the scope of the appended claims. The present invention is to be limited only by the terms of the appended claims, along with the full scope of equivalents to which such claims are entitled.

1. Use of calcitonin in the manufacture of a medicament for the treatment of a condition for which treatment with an anabolic agent is indicated.

2. The use of claim 1, wherein the condition is atherosclerosis.

3. The use of claim 1, wherein the calcitonin is salmon calcitonin.

4. Use of calcitonin in the manufacture of a medicament for the treatment of disorders of calcium metabolism in a selected patient population, wherein the patient population is selected on the basis of the gene expression profile indicative of calcitonin efficacy by the patient to whom calcitonin is administered.

5. The use of claim 4, wherein the calcitonin is salmon calcitonin.

6. The use of claim 4, where the calcitonin is administered in a therapeutic dose prior to determining the gene expression profile by the patient.

7. The use of claim 4, where the calcitonin is administered in a sub-therapeutic dose: prior to determining the gene expression profile by the patient.

8. Use of parathyroid hormone or a parathyroid hormone analogue in the manufacture of: a medicament for the treatment of disorders of calcium metabolism in a selected patient population, wherein the patient population is selected on the basis of the gene t expression profile indicative of parathyroid hormone or parathyroid hormone analogue efficacy by the patient to whom parathyroid hormone or parathyroid hormone analogue is administered.—70

9. The use of claim 8, wherein the parathyroid hormone analogue is PTS893.

10. The use of claim 8, where the parathyroid hormone or parathyroid hormone analogue is administered in a therapeutic dose prior to determining the gene expression profile by the patient.

11. The use of claim 8, where the parathyroid hormone or parathyroid hormone analogue is administered in a sub-therapeutic dose prior to determining the gene expression profile by the patient.

12. A method for treating a condition in a subject, wherein the condition is one for which administration of a calcitonin, parathyroid hormone, a parathyroid hormone analogue or a combination thereof is indicated, comprising the steps of: (a) administering a compound to the subject; (b) obtaining the gene expression profile of the subject, wherein the gene expression profile comprises the gene expression pattern of one or more genes, where the expression patterns of the one or more genes are a consequence of administration of the compound; and (c) comparing the gene expression profile of the subject to whom the compound was administered to a biomarker gene expression profile indicative of efficacy of treatment by a calcitonin, parathyroid hormone, a parathyroid hormone analogue or a combination thereof, wherein a similarity in the gene expression profile of the subject to whom the compound was administered to the biomarker gene expression profile is indicative of efficacy of treatment with the compound.

13. The method of claim 12, wherein the condition is one for which salmon calcitonin is indicated.

14. The method of claim 12, wherein the condition is one for which PTS893 is indicated.

15. The method of any one of claim 12, wherein the administered compound is a calcitonin, parathyroid hormone, a parathyroid hormone analogue or a combination thereof.

16. The method of claim 15, wherein the calcitonin is salmon calcitonin.

17. The method of claim 15, wherein the parathyroid hormone analogue is PTS893.

18. The method of claim 12, wherein the subject is a mammal.

19. The method of claim 18, wherein the mammal is a primate.

20. The method of claim 19, wherein the primate is a cynomolgus monkey or a human.

21. The method of claim 12, wherein the biomarker gene expression profile is the baseline gene expression profile of the subject before administration of the compound.

22. The method of claim 12, wherein the biomarker gene expression profile is the gene expression profile or average of gene expression profiles of a vertebrate to whom a calcitonin, parathyroid hormone, a parathyroid hormone analogue of a combination thereof has been administered.

23. The method of claim 12, wherein the gene expression profile comprises one or more genes selected from the group consisting of acid phosphatase 1 isoform a; activin A receptor type II like 1; activin A type IIB receptor precursor; activin beta C chain; alpha 2 HS glycoprotein; amelogenin; annexin V; arylsulfatase E precursor; ATPase H(+) vacuolar; ATPase H(+) vacuolar subunit; ATPase7 H+ transport, lysosomal; ATPase, H+ transporting, lysosomal; ATPase, H+ transporting, lysosomal; biglycan; bone morphogenetic protein 1; bone morphogenetic protein 10; bone morphogenetic protein 2A; bone morphogenetic protein 5; bone morphogenetic protein 6 precursor; calcium binding protein 1 (calbrain); calcium/calmodulin dependent protein kinase (CaM kinase) II gamma; calreticulin; cAMP responsive element modulator (CREM); carbonic anhydrase I; carbonic anhydrase 11; cartilage oligomeric matrix protein precursor; cathepsin K; cathepsin W.; CDC like kinase 1; CDC like kinase 2 isoform hclk2/139; chondroitin sulphate proteoglycan 2 (versican); chondroitin sulphate proteoglycan 3 (neurocan); chorionic somatomammotropin hormone 1; chymotrypsin C (caldecrin); collagen type 1 and PDGFB fusion transcript; collagen type II alpha 1; collagen type III alpha 1; collagen type IV alpha 2; collagen type IX alphal; collagen type VI alpha 1; collagen type VI alpha 2 (AA 570 99S); collagen type XI alpha 1; collagen type XI alpha2; collagen type XI alpha2; collagen, type I, alpha 2; collagen, type IV, alpha 1; collagen, type IX, alpha 2; collagen, type V, alpha 2; collagen, type VI, alpha 1; collagen, type VI, alpha 1 precursor; collagen, type XVI, alpha 1; collagen, type XVI, alpha 1; collagenase 3 (matrix metalloproteinase 13); connective tissue growth factor; cyclin A2; cyclin B1; cyclin D2; cyclin E2; cyclin dependent kinase 5; cyclin dependent kinase 5, regulatory subunit 1 (p35); cyclin dependent kinase 6; cyclin dependent kinase inhibitor 1A (p21, Cipl); cystatin B (stefin B); cytokine inducible kinase; death associated protein kinase 1; death associated protein kinase 3; dentin matrix acidic phosphoprotein 1 (DMP1); dual specificity phosphatase 9; dystrophia myotonica protein kinase; ectonucleotide pyrophosphatase/phosphodiesterase 1; ectonucleotide pyrophosphatase/phosphodiesterase 1; endothelial differentiation, G protein coupled receptor 6 precursor; oestrogen receptor; oestrogen receptor; oestrogen receptor related protein; oestrogen responsive B box protein (EBBP); fibroblast activation protein; fibroblast—73 growth factor 1 (acidic); fibroblast growth factor 18; fibroblast growth factor 4; fibroblast growth factor receptor; follistatin like 1; follistatin like 1; glutamate receptor, metabokopic 1; GPI1 N acetylglucosaminyl transferase component Gpil; granulocyte macrophage colony stimulating factor (CSF1); growth arrest and DNA damage inducible, alpha; growth factor receptor bound protein 10; heparan sulphate proteoglycan 2 (perlecan); inositol 1,4,5 triphosphate receptor, type 1; inositol 1,4,5 triphosphate receptor, type 1; inositol 1,4,5 triphosphate receptor, type 2; inositol 1,4,5 trisphosphate 3 kinase isoenzyme; inositol polyphosphate 4 phosphatase type I beta; inositol polyphosphate 5 phosphatase; inositol(myo) 1 (or 4) monophosphatase 1; inositol(myo) I(or 4) monophosphatase 2; insulin like growth factor (IGF H); insulin like growth factor 2 (somatomedin A); insulin like growth factor binding protein; insulin like growth factor binding protein 2; insulin like growth factor binding protein 3; insulin like growth factor binding protein 5; insulin like growth factor binding protein 2; insulin like growth factor II precursor; insulin like growth factor I[precursor; integrin alpha 10 subunit; interleukin 1 receptor associated kinase; Janus kinase 3; LIM protein (similar to rat protein kinase C binding enigma); lysyl oxidase like protein; MAD, mothers against decapentaplegic homolog 3; MAGUKs (membrane associated guanylate kinase homologues; MAP kinase kinase kinase (MTK1); MAPK13: mitogen activated protein kinase 13; MAPK8IP1: mitogen activated protein kinase 8 interacting protein 1; MEK kinase; metalloproteinase; mitogen activated protein kinase 1; mitogen activated protein kinase 8; mitogen activated protein kinase kinase 1; mitogen activated protein kinase kinase kinase kinase 4; mitogen activated protein kinase activated protein kinase 2; mitogen activated protein kinase activated protein kinase 3; MMD: monocyte to macrophage differentiation associated; neurochondrin; nuclear factor of activated T cells, cytoplasmic, calcineurin dependent 1; OS 4 protein (OS 4); OSF 20s osteoblast specific factor 2 (periostin); osteoclast stimulating factor (OSF); PAK4; PDGF associated protein; phosphatidylinositol 4 kinase, catalytic, beta polypeptide; phosphatidylinositol glycan, class L; phosphatidylinositol polyphosphate 5 phosphatase, isoform b; phosphatidylinositol 4 phosphate 5 kinase isoform C (1); phosphatidylinositol 4 phosphate 5 kinase, type 1, beta; phosphatidylinositol 4-74 phosphate 5 kinase, type II, beta; phosphatidylinositol glycan class C (PIG C); phosphodiesterase 4A, cAMP specific; phosphodiesterase 4D, cAM:P specific (dunce (Drosophila) homolog phosphodiesterase E3); phosphodiesterase IB, calmodulin dependent; phosphoinositide 3 kinase; phosphoinositide 3 kinase, catalytic, gamma polypeptide; phosphoinositide 3 kinase, class 3; phospholipase C b3; phospholipase C, beta 4; phospholipase D; phosphotidylinositol transfer protein; PKD2 Protein kinase D2; preprocollagen type I alpha 2; preprocollagen type I alphal; procollagen alpha 1 type II; procollagen lysine 5 dioxygenase; procollagen praline, 2 oxoglutarate 4 dioxygenase (proline 4 hydroxylase), alpha polypeptide I; progestagen associated endometrial protein (placental protein 14, pregnancy associated endometrial alpha 2 globulin, alpha uterine protein); prolidase (imidodipeptidase) PEPD; proliferating cell nuclear antigen; prolyl 4 hydroxylase beta; protease, serine, 11 (IGF binding); proteasome (prosome, macropain) subunit, beta type, 10; protein inhibitor of activated STAT X; protein kinase 1 PCTAIRE; protein kinase-C substrate 80K H.; protein kinase C, alpha; protein kinase, cAMP dependent, catalytic, gamma; protein kinase, cAMP dependent, regulatory, type I, beta; protein kinase, cAMP-dependent, regulatory, type II, alpha; purinergic receptor P2Y, G protein coupled, 11; RAC2 Ras related C3 botulinum toxin substrate 2 (rho family, small GTP binding protein Rac2); receptor tyrosine kinase DDR; retinoid X receptor gamma; ribosomal protein S6 kinase; ribosomal protein S6 kinase, 90 kD, polypeptide 3; SCAMP1: secretory carrier membrane protein 1 (vesicular transport); secreted phosphoprotein 1 (osteopontin, bone sialoprotein I, early T lymphocyte activation 1); serine (or cysteine) proteinase inhibitor, clade H (heat shock protein 47), member 2; serine/threonine kinase 38; serine/threonine protein kinase; SF 1; Steroidogenic factor 1; signal transducer and activator of transcription 1; signal transducer and activator of transcription 2, 113 kD; signal transducer and activator of transcription 5A; signal transducer and activator of transcription 5A; signal transducer and activator of transcription 6 (STAT6); Smad 3; Smad anchor for receptor activation, isoform 1; Smad5; SMAD6 (inhibits BMP/Smadl (MADH1); SNF 1 related kinase; Spi B transcription factor (Spi 1/PU. 1 related); Stat5b (stat5b); Ste20 related serine/threonine kinase; TEIG; TGFB inducible early growth response; TGFB inducible early growth response; TEG; TGFB 1 induced—75 anti apoptotic factor 1; TGF beta induced apoptosis protein 12; TGF beta precursor; TGF beta superfamily protein; Tob; tousled like kinase 1; transforming growth factor, beta receptor m (betaglycan, 300 kD); transforming growth factor beta 3 (TGF beta 3); TRIO: triple functional domain (PTPRF interacting); tubulin alpha 1; tubulin alpha 3; tubulin alpha isotype H2 alpha; tubulin beta 2; tubulin beta 3; tubulin beta 4; tubulin beta, cofactor D; type VI collagen alpha 2 chain precursor; ubiquitin carrier protein E2 C; vascular endothelial growth factor; vascular endothelial growth factor; vascular endothelial growth factor B; and Y box binding protein 1.

24. The method of claim 23, wherein the gene expression profile comprises an increase in one or more genes selected from the group consisting of bone morphogenetic protein 5; cartilage oligomeric matrix protein; cathepsin K; pre-pro-alpha-2 type I collagen; and Y-box binding protein (bone and kidney).

25. The method of claim 23, wherein the gene expression profile in bone comprises a decrease in one or more genes selected from the group consisting of carbonic anhydrase II; Spi-B; and Y-box binding protein (muscle).

26. The method of claim 23, wherein the gene expression profile in bone comprises one or more genes selected from the group consisting of PU.1 (SPIT; Spi-B); granulocyte to macrophage colony-stimulating factor (CSF1) and monocyte to macrophage differentiation associate (MMD).

27. The method of claim 23, wherein the gene expression profile in bone comprises a change in the expression of osteoclast stimulating factor (OSF).

28. The method of claim 23, wherein the gene expression profile in bone comprises a change in the expression of vascular endothelial growth factor (VEGF).—76

29. The method of claim 23, wherein the gene expression profile in bone comprises a change in the expression of a gene selected from the group consisting of integrins; collagenase; matrix metalloproteinases I and 1:I; procollagen endopeptidase/proteinase; lysyl hydroxylase; aggrecan; cartilage oligomeric matrix protein precursor; collagens type 1, type II, type m, type IV, type V, type VI, type IX, type X, type XI, type xm, type XIV, type XV, and type XVI; chondroitin sulphate proteoglycan; dermatopontin; heparan sulphate proteoglycan; and syndecan.

30. The method of claim 23, wherein the gene expression profile in bone comprises a change in the expression of a gene selected from the group consisting of arnelogenin; dentin; ectonucleotide pyrophosphatases; and VEGF.

31. A method for choosing subjects for inclusion in a clinical trial for determining the efficacy of a compound for efficacy of treatment of a condition, wherein the condition is one for which administration of a calcitonin, parathyroid hormone, a parathyroid hormone, analogue or a combination thereof is indicated, comprising the steps of: (a) administering the compound to the subject; (b) obtaining the gene expression profile of the subject, wherein the gene expression profile comprises the gene expression pattern of one or more genes, where the expression patterns of the one or more genes are a consequence of administration of the compound; (c) comparing the gene expression profile of the subject to whom the compound was administered to a biomarker gene expression profile; and. (d) then: (i) including the subject in the clinical trial when the gene expression profile of the subject to whom the compound was administered is similar to the biomarker gene expression profile indicative of efficacy of treatment by a calcitonin, parathyroid hormone, a parathyroid hormone analogue or a combination thereof; or (ii) excluding the subject from the clinical trial when the gene expression profile of the subject to whom the compound was administered is dissimilar to the biomarker gene expression profile indicative of efficacy of treatment by a calcitonin, parathyroid hormone, a parathyroid hormone analogue or a combination thereof.

32. The method of claim 31, wherein the compound is administered to the subject at a sub-therapeutic dose.

33. A method for determining whether a compound has a therapeutic efficacy similar to that of calcitonin, comprising the steps of: (a) administering the compound to the subject; (b) obtaining the gene expression profile of the subject, wherein the gene expression profile comprises the gene expression pattern of one or more genes, where the expression patterns of the one or more genes are a consequence of administration of the compound; (c) comparing the gene expression profile of the subject to whom the compound was administered to a biomarker gene expression profile indicative of efficacy of treatment by calcitonin; and (d) then: (i) determining that the compound has a therapeutic efficacy similar to that of calcitonin when the gene expression profile of the subject to whom the compound was administered is similar to the biomarker gene expression profile of a subject to whom calcitonin is administered; or (ii) determining that the compound has a therapeutic efficacy different from that of calcitonin when the gene expression profile of the subject to whom the compound was administered is different from the biomarker gene expression profile of a subject to whom calcitonin is administered.

34. The method of claim 33, wherein the calcitonin is salmon calcitonin.

35. The method of claim 33, wherein the subject is a mammal.

36. The method of claim 35, wherein the mammal is a primate.

37. The method of claim 36, wherein the primate is a cynomolgus monkey or a human.

38. The method of any one of claims 33, wherein the compound is administered to the subject at a sub-therapeutic dose.

39. A method for determining whether a compound has a therapeutic efficacy similar to that of a parathyroid hormone analogue, comprising the steps of: (a) administering the compound to the subject; (b) obtaining the gene expression profile of the subject, wherein the gene expression profile comprises the gene expression pattern of one or more genes, where the expression patterns of the one or more genes are a consequence of administration of the compound; (c) comparing the gene expression profile of the subject to whom the compound was administered to a biomarker gene expression profile indicative of efficacy of treatment by a parathyroid hormone analogue; and (d) then: (i) determining that the compound has a therapeutic efficacy similar to that of a parathyroid hormone analogue when the gene expression profile of the subject to whom the compound was administered is similar to the biomarker gene expression profile of a subject to whom a parathyroid hormone analogue is administered; or (ii) determining that the compound has a therapeutic efficacy different from that of a parathyroid hormone analogue when the gene expression profile of the subject to whom the compound was administered is different from the biomarker gene expression profile of a subject to whom a parathyroid hormone analogue is administered.

40. The method of claim 39, wherein the parathyroid hormone analogue is PTS893.

41. The method of claim 39, wherein the subject is a mammal.

42. The method of claim 41, wherein the mammal is a primate.

43. The method of claim 42, wherein the primate is a cynomolgus monkey or a human.

44. The method of claim 39, wherein the compound is administered to the subject at a sub therapeutic dose.

45. A kit for use in determining treatment efficacy of a condition for which administration of a calcitonin, parathyroid hormone or a parathyroid hormone analogue is indicated, comprising: (a) a reagent for detecting a biomarker of treatment efficacy of a condition for which administration of a calcitonin, parathyroid hormone or a parathyroid hormone analogue is indicated; (b) a container for the reagent; and (c) a written product on or in the container describing the use of the biomarker in determining the treatment strategy of the condition.

46. The kit of claim 45, wherein the reagent is a gene chip.

47. The kit of claim 45, wherein the reagent is a hybridization probe.

48. The kit of claim 45, wherein the reagent is a gene amplification reagent.

49. The kit of any one of claims 45, wherein the biomarker comprises one or more genes selected from the group consisting of acid phosphatase 1 isoform a; activin A receptor type II like 1; activin A type mB receptor precursor; activin beta C chain; alpha 2 US glycoprotein; amelogenin; annexin V; arylsulfatase E precursor; ATPase H(+) vacuolar; ATPase H(+) vacuolar subunit; ATPase, H+ transport, lysosomal; ATP ase, H+ transporting, lysosomal; ATP as e, H+ transporting, lyso somal; biglyc an; bone morphogenetic protein 1; bone morphogenetic protein 10; bone morphogenetic protein 2A; bone morphogenetic protein 5; bone morphogenetic protein 6 precursor; calcium binding protein 1 (calbrain); calcium/calmodulin dependent protein kinase (CaM kinase) It gamma; calreticulin; cAMP responsive element modulator (CREM); carbonic anhydrase I; carbonic anhydrase II; cartilage oligomeric matrix protein precursor; cathepsin K; cathepsin W; CDC like kinase 1; CDC like kinase 2 isoform hclk2/139; chondroitin sulphate proteoglycan 2 (versican); chondroitin sulphate proteoglycan 3 (neurocan); chorionic somatomammotropin hormone 1; chymotrypsin C (caldecrin); collagen type 1 and PDGFB fusion transcript; collagen type II alpha 1; collagen type m alpha 1; collagen type IV alpha 2; collagen type IX alphal; collagen type VI alpha 1; collagen type VI alpha 2 (AA 570 998); collagen type XI alpha,1; collagen type XI alpha2; collagen type XI alpha2; collagen, type I, alpha 2; collagen, type IV, alpha 1; collagen, type IX, alpha 2; collagen, type V, alpha 2; collagen, type VI, alpha 1; collagen, type VI, alpha 1 precursor; collagen, type XVI, alpha 1; collagen, type XVI, alpha 1; collagenase 3 (matrix metalloproteinase 13); connective tissue growth factor; cyclin A2; cyclin B1; cyclin D2; cyclin E2; cyclin dependent kinase 5; cyclin dependent kinase 5, regulatory subunit 1 (p35); cyclin dependent kinase 6; cyclin dependent kinase inhibitor 1A (p21, Cipl); cystatin B (stefin B); cytokine inducible kinase; death associated protein kinase 1; death associated protein kinase 3, dentin matrix acidic phosphoprotein 1 (DMP1); dual specificity phosphatase 9; dystrophia myotonica protein kinase; ectonucleotide Pyrophosphatase/Phosphodiesterase 1; ectonucleotide pyrophosphatase/phosphodiesterase 1; endothelial differentiation, G protein coupled receptor 6 precursor; oestrogen receptor; oestrogen receptor; oestrogen receptor related protein; oestrogen responsive B box protein (EBBP); fibroblast activation protein; fibroblast growth factor 1 (acidic);—82 fibroblast growth factor 18; fbroblast growth factor 4; fibroblast growth factor receptor; follistatin like 1; follistatin like 1; glutamate receptor, metabotropic 1; GPI1 N acetylglucosaminyl transferase component Gpil; granulocyte macrophage colony stimulating factor (CSF1); growth arrest and DNA damage inducible, alpha; growth factor receptor bound protein 10; heparan sulphate proteoglycan 2 (perlecan); inositol 1,4,5 triphosphate receptor, type 1; inositol 1,4,5 triphosphate receptor, type 1; inositol 1,4,5 triphosphate receptor, type 2; inositol 1,4,5 trisphosphate 3 kinase isoenzyme; inositol polyphosphate 4 phosphatase type I beta; inositol polyphosphate 5 phosphatase; inositol(myo) I(or 4) monophosphatase 1; inositol(myo) I(or 4) monophosphatase 2; insulin like growth factor (IGF II); insulin like growth factor 2 (somatomedin A); insulin like growth factor binding protein; insulin like growth factor binding protein 2; insulin like growth factor binding protein 3; insulin like growth factor binding protein 5; insulin like growth factor binding protein 2; insulin like growth factor II precursor; insulin like growth factor II precursor; integrin alpha 10 subunit; interleukin 1 receptor associated kinase; Janus kinase 3; LIM protein (similar to rat protein kinase C binding enigma); lysyl oxidase like protein; MAD, mothers against decapentaplegic homolog 3; MAGUKs (membrane associated guanylate kinase homologues; MAP kinase kinase kinase (MTK1); MAPK13: mitogen activated protein kinase 13; MAPK81P1: mitogen activated protein kinase 8 interacting protein 1; MEK kinase; metalloproteinase; mitogen activated protein kinase 1; mitogen activated protein kinase 8; mitogen activated protein kinase kinase 1; mitogen activated protein kinase kinase kinase kinase 4; mitogen activated protein kinase activated protein kinase 2; mitogen activated protein kinase activated protein kinase 3; MMD: monocyte to macrophage differentiation associated; neurochondrin; nuclear factor of activated T cells, cytoplasmic, calcineurin dependent 1; OS 4 protein (OS 4); OSF 20s osteoblast specific factor 2 (periostin),; osteoclast stimulating factor (OSF); PAK4; PDGF associated protein; phosphatidylinositol 4 kinase, catalytic, beta polypeptide; phosphatidylinositol glycan, class L; phosphatidylinositol polyphosphate 5 phosphatase, isoform b; phosphatidylinositol 4 phosphate 5 kinase isoform C (1); phosphatidylinositol 4 phosphate 5 kinase, type I, beta; phosphatidylinositol 4 phosphate 5 kinase, type II, beta; phosphatidylinositol glycan class C (PIG C);—83 phosphodiesterase 4A, cAMP specific; phosphodiesterase 4D, cAMP specific (dunce (Drosophila) homolog phosphodiesterase E3); phosphodiesterase IB, calmodulin dependent; phosphoinositide 3 kinase; phosphoinositide 3 kinase, catalytic, gamma polypeptide; phosphoinositide 3 kinase, class 3; phospholipase C b3; phospholipase C, beta 4; phospholipase D; phosphotidylinositol transfer protein; PKD2 Protein kinase D2; preprocollagen type I alpha 2; preprocollagen type I alphal; procollagen alpha 1 type II; procollagen lysine 5 dioxygenase; procollagen proline, 2 oxoglutarate 4 di oxygenase (pro line 4 hydroxyl as e), alpha polyp eptide I; pro gestagen as soci ated endometrial protein (placental protein 14, pregnancy associated endometrial alpha 2 globulin, alpha uterine protein); prolidase (imidodipeptidase) PEPD; proliferating cell nuclear antigen; prolyl 4 hydroxylase beta; protease, serine, 11 (IGF binding); proteasome (prosome, macropain) subunit, beta type, 10; protein inhibitor of activated STAT X; protein kinase 1 PCTAIRE; protein kinase C substrate 80K H; protein kinase C, alpha; protein kinase, cAMP dependent, catalytic, gamma; protein kinase, cAMP dependent, regulatory, type I, beta; protein kinase, cAMP dependent, regulatory, type II, alpha; purinergic receptor P2Y, G protein coupled, 11; RAC2 Ras related C3 botulinum toxin substrate 2 (rho family, small GTP binding protein Rac2); receptor tyrosine kinase DDR; retinoid X receptor gamma; ribosomal protein S6 kinase; ribosomai protein S6 kinase, 90 kD, polypeptide 3; SCAMP1: secretory carrier membrane protein 1 (vesicular transport); secreted phosphoprotein 1 (osteopontin, bone sialoprotein I, early T lymphocyte activation 1); serine (or cysteine) proteinase inhibitor, clade H (heat shock protein 47), member 2; serine/threonine kinase 38; serine/threonine protein kinase; SF 1; Steroidogenic factor 1; signal transducer and activator of transcription 1; signal transducer and activator of transcription 2, 113 kD; signal transducer and activator of transcription 5A; signal kansducer and activator of kanscription 5A; signal transducer and activator of kanscription 6 (STAT6); Smad 3; Smad anchor for receptor activation, isoform 1; Smad5; SMAD6 (inhibits BMP/Smadl (MADH1); SNF 1 related kinase; Spi B transcription factor (Spi 1/PU.1 related); Stat5b (stat5b); Ste20 related serine/threonine kinase; TEIG; TGFB inducible early growth response; TGFB inducible early growth response; TEG; TGFB1 induced anti apoptotic factor 1; TGF beta induced apoptosis protein 12; TGF beta precursor;—84 TGF beta superfamily protein; Tob; tousled like kinase 1; transforming growth factor, beta receptor m (betaglycan, 300 kD); transforming growth factor beta 3 (TGF beta 3); TRIO: triple functional domain (PTPRF interacting); tubulin alpha 1; tubulin alpha 3; tubulin alpha isotype H2 alpha; tubulin beta 2; tubulin beta 3; tubulin beta 4; tubulin beta, cofactor D; type VI collagen alpha 2 chain precursor; ubiquitin carrier protein E2 C; vascular endothelial growth factor; vascular endothelial growth factor; vascular endothelial growth factor B.; and Y box binding protein 1.
